(19) United States
(12) Patent Application Publication
(10) Pub. No.: US 2016/0074400 A1
(43) Pub. Date: Mar. 17, 2016

(54) WNT MODULATORS FOR THE PROTECTION, MITIGATION AND TREATMENT OF RADIATION INJURY

(71) Applicants: University of Southern California, Los Angeles, CA (US); Albert Einstein College of Medicine of Yeshiva University, Bronx, NY (US)

(72) Inventors: Michael Kahn, Altadena, CA (US); Chandan Guha, Scarsdale, NY (US); Subhrajit Saha, Bronx, NY (US); Payel Bhanja, Bronx, NY (US)

(21) Appl. No.: 14/419,646

(22) PCT Filed: Aug. 6, 2013
(86) PCT No.: PCT/US13/53867

§ 371 Date: Feb. 4, 2015

Related U.S. Application Data
(60) Provisional application No. 61/680,161, filed on Aug. 6, 2012.

(51) Int. Cl. A61K 31/519; A61K 38/17; C12N 7/00; A61K 45/06

(52) U.S. Cl. CPC A61K 31/519; A61K 45/06; A61K 38/1709; C12N 7/00; C12N 2710/10043; C12N 2710/10071

(57) ABSTRACT

Provided are composition and methods for treating radiation-induced cell damage, and in particular aspects to methods for protecting, mitigating or otherwise treating radiation-induced induced depletion of tissue stem cells and injury to the supportive stem cell niche, and in even more particular aspects to methods for protecting, mitigating or otherwise treating radiation-induced gastrointestinal syndrome (RIGS), comprising sequential administration of a Wnt pathway activator/agonist (e.g., Rspo1 or a small molecule inducer thereof) that amplifies the surviving intestinal stem cell (ISC) pool post-radiation exposure, followed by a selective antagonist of the β-Catenin-CBP interaction (e.g., ICG-001 or other exemplary compounds disclosed herein) that accelerates differentiation of the stimulated (e.g., Rspo1-stimulated) ISC in crypt-villi axis, promoting villous regeneration and intestinal restitution, thereby mitigating RIGS. Adjunctive and combination therapy embodiments are encompassed.

### CROSS-REFERENCE TO RELATED APPLICATIONS

This application claims the benefit of copending U.S. Provisional Patent Application No. 61/680,161, filed on Aug. 6, 2012, and entitled “WNT MODULATORS FOR THE PROTECTION, MITIGATION AND TREATMENT OF RADIATION INJURY,” which is incorporated herein by reference in its entirety.

### STATEMENT REGARDING FEDERALLY-SPONSORED RESEARCH

This invention was made with government support under Contract Nos. NIAID 1 RC2 AI087612-01 and NIAID 1U19 AI091175-01 awarded by the National Institute of Allergy and Infectious Diseases (NIAID). The government has certain rights in the invention.

### FIELD OF THE INVENTION

Aspects of the invention relate generally to radiation-induced cell damage, and in more particular aspects to methods for protecting against, mitigating or otherwise treating radiation-induced depletion of tissue stem cells and injury to the supportive stem cell niche, and in even more particular aspects to methods for protecting against, mitigating or otherwise treating radiation-induced gastrointestinal syndrome (RIGS), comprising sequential administration of a Wnt pathway activator (e.g., R-spondin1 (R-spo1), small molecule inducers of Rspo1, direct Wnt/catenin activators, LiCl, GSK3-beta inhibitors, CHIR (e.g., CHIR-911), arylhydrdocarbon receptor (AHR) agonists, beta napthoflavone, indole-3-carbinol (I3C), high-affinity AhR ligands (DIM and ICZ), formylindolo[3,2-b]carbazoles, 6-formylindolo[3,2-b]carbazole (FICZ) FICZ-derived indolo[3,2-b]carbazole-6-carboxylic acid metabolites and sulfoconjugates, which induce the expression of Rspo1) that amplifies the surviving intestinal stem cell (ISC) pool post-radiation exposure, followed by a selective antagonist of the β-Catenin-CBP interaction, as disclosed herein, that accelerates differentiation of the stimulated (e.g., Rspo1-stimulated) ISC in crypt-villi axis, promoting villous regeneration and intestinal restitution, thereby mitigating RIGS. Adjunctive and combination therapy embodiments are encompassed.

### SEQUENCE LISTING

A Sequence Listing (in .txt format) comprising SEQ ID NOS:1-12 was filed as part of this application, and is incorporated by reference herein in its entirety.

### BACKGROUND OF THE INVENTION

Radiation injury is caused by radiation-induced depletion of tissue stem cells and injury to the supportive stem cell niche. For example, radiation-induced gastrointestinal syndrome (RIGS) results from a combination of loss of crypt progenitors and stromal cells along with aberrant signaling in the intestinal stem cell (ISC) niche (and also damage to the normal intestinal epithelium), while bone marrow syndrome is caused by depletion of hematopoietic stem cells (and depletion of normal hematopoietic cells in general e.g. neutrophils, platelets), as radiation damage to the more differentiated cells with a lack of or too limited a response to the injury and depletion of the stem cell niche may be the generic problem.

High doses of radiation induce damage to various rapidly regenerating organ systems (gut, hematopoietic system) and their resident stem cell populations. Regeneration of these organ systems after radiation toxicity requires both proliferation of the resident stem cell populations and subsequent differentiation of the stem cells to give differentiated tissues.

Accidental or intended radiation exposure in a mass casualty setting presents a serious and ongoing threat. In addition to potential nuclear accidents and terrorism, unintended injury can also occur during radiation therapy of cancer patients because of increased radiosensitivity of individual patients or misadministration of radiation dose due to machine malfunction or faulty dose calculation.

Acute radiation injury is manifested in organs that have rapidly proliferating cells, such as, intestine, mucosal lining of the body, bone marrow and skin. Manifestation of acute radiation injury includes anemia, bleeding, diarrhea, sepsis, mucosal and cutaneous ulceration and even death due to target organ failure. There are currently no approved treatments to alleviate Acute Radiation Syndrome (ARS) in victims of radiological disaster with anticipated multi-organ failure or to effectively treat/protect first responders from ARS. To date, individuals categorized as H4 (dose and volume irradiation dependent) receive supportive care post-radiation exposure that includes reverse isolation, antibiotics, antivirals, antifungals, platelet and blood transfusions and maintenance of fluid/electrolyte balance prior to bone marrow transplantation (BMT), resulting in only marginal survival. While radioprotective agents can be used with some success when given prior to radiation exposure they are of limited use when used post-exposure. This circumstance motivates the continued search for agents that alleviate radiation damage post-exposure.

Late radiation injury is manifested in organs that have parenchymal cells that divide slowly, such as, brain, spinal cord and liver. In addition, chronic radiation injury can occur in any organ, including lung, kidney, intestine, esophagus, bladder and rectum. Chronic radiation injury is caused by aberrant repair of acute radiation injury and is usually seen as a fibrotic response.

Syndromes and symptoms that are caused by radiation injury include xerostomia (dry mouth), dysphagia (difficulty in swallowing) due to pharyngeal and esophageal strictures, breast fibrosis, cutaneous ulcers, dyspnea due to radiation pneumonitis and lung fibrosis, radiation-induced liver damage, kidney failure due to fibrotic kidneys, rectal bleeding due to radiation proctitis, bladder and urethral injury, diarrhea, enteric bleeding and sepsis due to radiation-induced gastrointestinal syndrome, and anemia, thrombocytopenia and neutropenia from radiation-induced marrow failure. Basically most organs can manifest some form of acute or chronic radiation injury.

Bone marrow transplantation (BMT) can rescue acute radiation injury in victims exposed to 6-8 Gy of irradiation by providing hematopoietic stem cells to rescue and replace the irradiated marrow. However, patients usually die with exposure to higher dose of irradiation because of lethal injury to other organs, such as, intestine and lung, and cannot be rescued by BMT alone.

While transplantation of mesenchymal stem cells (MSC) has been used to facilitate intestinal regeneration in the context of graft versus host disease, ulcer and colitis (e.g., to ameliorate intestinal injury in murine models of radiation and chemotherapy-induced injury, colitis, and autoimmune enteropathy), transplantation of whole bone marrow or MSC has neither been successful in ameliorating radiation-induced gastrointestinal syndrome (RIGS) nor in improving survival of mice that received >10 Gy of irradiation in a single fraction.

Radioprotectants, such as, amifostine and Toll like receptor (TLR) agonist can ameliorate radiation injury to the intestine. Additionally, R-spondin1 (a Wnt agonist) can protect intestine from radiation toxicity after lethal whole body irradiation (Bhanja et al. PLoS One 2009; 4:e8014). Moreover, activation of Toll like receptor 5 could also protect intestine from lethal irradiation (Lyudmila et al. Science, 2008, Vol. 320, 226-230).

There are currently, however, no approved therapeutic treatments to effectively protect first responders from Acute Radiation Syndrome (ARS) or to alleviate ARS in victims of radiological disaster. To date, only Ethyol (Amifostine) has been approved by the FDA for clinical radioprotection of salivary glands, when used prior to exposure to external beam radiation therapy for H&N cancer. Most post-event strategies associated with ARS have been severely limited to within only several hours of the event (with the exception of bone marrow transplantation) and have demonstrated only marginal protection. As such, there is no known post-exposure strategy to rescue/salvage critical biological elements of RIGS within days after the radiation event has occurred. There is no adequate treatment for radiation injury to intestine and lung, and no mitigating or therapeutic agents available to combat the consequences of lethal radiation injury due to RIGS.

### SUMMARY OF THE INVENTION

According to particular aspects, sequential therapy with Wnt pathway activator (e.g., R-spondin1 (R-spo1), small molecule inducers of Rspo1, direct Wnt/catenin activators, LiCl, GSK3-beta inhibitors, CHIR (e.g., CHIR-911), arylhydrdocarbon receptor (AHR) agonists, beta napthoflavone, indole-3-carbinol (I3C), high-affinity AhR ligands DIM and ICZ), formylindolo[3,2-b]carbazoles, 6-formylindolo[3,2-b]carbazole (FICZ) FICZ-derived indolo[3,2-b]carbazole-6-carboxylic acid metabolites and sulfoconjugates, which induce the expression of Rspo1) followed by a differentiating agent (e.g., ICG-001) can mitigate RIGS (e.g., induced by whole body irradiation (≦10.4 Gy)).

According to particular aspects of the present invention, a sequential administration of a Wnt pathway agonist/activator (e.g., R-spondin1 (R-spo1), small molecule inducers of Rspo1, direct Wnt/catenin activators, LiCl, GSK3-beta inhibitors, CHIR (e.g., CHIR-911), aryl-hydrocarbon receptor (AHR) agonists, beta napthoflavone, indole-3-carbinol (I3C), high-affinity AhR ligands DIM and ICZ), formylindolo[3,2-b]carbazols, 6-formylindolo[3,2-b]carbazole (FICZ) FICZ-derived indolo[3,2-b]carbazole-6-carboxylic acid metabolites and sulfoconjugates, which induce the expression of Rspo1) that amplifies the surviving ISC pool post-radiation exposure, followed by a selective antagonist of the β-Catenin-CBP interaction (e.g., ICG-001) that accelerates differentiation of Rspo1-stimulated ISC in crypt-villi axis, promotes villous regeneration and intestinal restitution, thereby mitigating RIGS. Particular exemplary references on Rspo receptors are Lau et al. (Nature, doi:10.1038/nature10337, 2011), and Carmon et al. (Mol. Cell. Biol. 2012, 32(11):2054. DOI:10.1128/MCB.00272-12, 2 Apr. 2012), which are incorporated by reference herein in their entireties.

In particular aspects, sequential administration of a recombinant adenovirus expressing R-spondin1 (within 24 hours post exposure) with ICG-001 (72 hrs post-exposure), a CBP/catenin antagonist, mitigated radiation injury and improved survival in mice that were exposed to lethal doses of whole body irradiation (10.4 Gy in single fraction), thus providing the first demonstration to Applicants' knowledge of mitigation for acute radiation injury with small molecular Wnt modulators.

### Exemplary Preferred Aspects:

Particular aspects provide methods for protecting, mitigating or otherwise treating radiation-induced depletion of tissue stem cells, comprising: identifying a mammalian subject having, suspected to have, or expected to receive, radiation-induced depletion of somatic stem cells for least one tissue compartment or type; administering to the subject a Wnt pathway agonist/activator in an amount sufficient to stimulate amplification of the surviving somatic stem cell pool for the at least one tissue compartment; and administrating to the subject, administering the Wnt pathway agonist/activator, an amount of a CBP/catenin antagonist sufficient to promote differentiation of the amplified somatic stem cells, wherein a method for protecting, mitigating or otherwise treating radiation-induced depletion of the somatic stem cells for the at least one tissue compartment or type is afforded. In certain aspects, the exposure to radiation is at least 6 Gy, at least 7 Gy, at least 8 Gy, at least 9 Gy, or at least 10 Gy. In particular embodiments, the somatic stem cells comprise at least one selected from the stem cell group consisting of skin including keratinocyte stem cells, epidermal, follicular, hematopoietic, mammary, intestinal epithelium including crypt cells, intestinal stem cell (ISC), mesenchymal including muscle satellite cells, melanocyte stem cells, osteoblasts and chondrocyte progenitors, endothelial, neural, including either the ependymal or the subventricular zone cells, neural crest, olfactory, testicular, uterine, lung, and cuticle stem cells. In certain embodiments, the somatic stem cells comprise intestinal stem cells (ISC). Particular embodiments of the methods comprise amplifying the surviving intestinal stem cell (ISC) pool, followed by accelerated differentiation of the amplified ISC in crypt-villi axis, providing for villous regeneration and intestinal restitution. In preferred aspects, treating radiation-induced gastrointestinal syndrome (RIGS) is afforded. In particular aspects, the somatic stem cells comprise skin stem cells of a skin tissue compartment or type, wherein enhanced post-radiation skin repair and/or homeostatic maintenance is provided at the skin tissue compartment or type. In particular aspects, the somatic stem cells for the at least one tissue compartment or type comprise quiescent somatic stem cells, wherein administering the CBP/catenin antagonist comprises CBP/catenin antagonist-mediated activation of the quiescent somatic stem cells to enhance or accelerate asymmetric renewing divisions relative to, or at the expense of symmetric divisions among the somatic stem cells of the at least one tissue compartment or type. In certain aspects, administration of either or both of the Wnt pathway activator/agonist or/and the CBP/β-catenin antagonist comprises at least one of oral, intravenous, intramuscular, topical, gingival, buccal, and sub cutaneous administration.

In particular embodiments of the methods, the Wnt pathway agonist/activator is at least one selected from the group consisting of R-spondin1 (R-spo1), direct Wnt/catenin activators, LiCl, GSK3-beta inhibitors including CHIR (e.g., CHIR-911), small molecule inducers of Rspo1, arylhydrdocarbon receptor (AHR) agonists, beta napthoflavone, indole-3-carbinol (I3C), high-affinity AhR ligands DIM and ICZ), formylindolo[3,2-b]carbazoles, 6-formylindolo[3,2-b]carbazole (FICZ) FICZ-derived indolo[3,2-b]carbazole-6-carboxylic acid metabolites and sulfoconjugates, which induce the expression of Rspo1.

In particular embodiments of the methods, the CBP/catenin antagonist is at least one selected from the group of compounds and salts thereof of Table 1, or another compound disclosed herein. In certain aspects, the CBP/β-catenin antagonist comprises ICG-001 or an active derivative or variant thereof.

Particular embodiments of the methods, comprise co-administration of or adjunct treatment with at least one other therapeutic agent. Certain of such aspects comprise simultaneously or adjunctively treating the subject with at least one anti-inflammatory agent or antiviral agent. In particular embodiments, said at least one anti-inflammatory agent comprises a steroid or glucocorticoid steroid. In certain embodiments, the at least one anti-inflammatory agent is selected from the group consisting of: short-acting β₂-agonists, long-acting β₂-agonists, anticholinergics, corticosteroids, systemic corticosteroids, mast cell stabilizers, leukotriene modifiers, methylxanthines, β₂-agonists, albuterol, levalbuterol, pirbuterol, artformoterol, formoterol, salmeterol, anticholinergics including ipratropium and tiotropium; corticosteroids including beclomethasone, budesonide, flunisolide, fluticasone, mometasone, triamcinolone, methyprednisolone, prednisolone, prednisone; leukotriene modifiers including montelukast, zafirlukast, and zileuton; mast cell stabilizers including cromolyn and nedocromil; methylxanthines including theophylline; combination drugs including ipratropium and albuterol, fluticasone and salmeterol, glucocorticoid steroids, budesonide and formoterol; antihistamines including hydroxyzine, diphenhydramine, loratadine, cetirizine, and hydrocortisone; immune system modulating drugs including tacrolimus and pimecrolimus; cyclosporine; azathioprine; mycophenolatemofetil; and combinations thereof.

In certain aspects, the one additional therapeutic agent is selected from the group consisting of anti-microbial agents, antifungal agents, and antibiotic agents. In particular embodiments, the at least one additional therapeutic agent is selected from the group consisting of: ciclosporin, hyaluronic acid, carmellose, macrogol(s), dextran and hyprolose, sodium and calcium, sodium and povidone, hypromellose, carbomer, amikacin, gentamicin, kanamycin, neomycin, netilmicin, streptomycin, tobramycin, paromomycin, geldanamycin, herimycin, loracarbef, ertapenem, imipenem/cilastatin, meropenem, cefadroxil, cefazolin, cefalotin/cefalothin, cephalexin, cefaclor, cefamandole, cefoxitin, cefuroxime, cefixime, cefdinir, cefditoren, cefoperazone, cefotaxime, cefpodoxime, ceftazidime, ceftibuten, ceftizoxime, ceftriaxone, cefeprime, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, nafcillin, penicillin, peperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, protosil, sulfacetamide, sulfamethizole, sulfanilamide, sulfasalazine, sulfisoxazole, trimethoprim, trimethoprim-sulfamethoxazole, demeclocycline, doxycycline, minocycline, oxytetracycline, tetracycline, arsphenamine, chloramphenicol, clindamycin, lincoamycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin/rifampicin, tinidazole, miconazole, ketoconazole, clotrimazole, econazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, teronazole, terbinafine, amorolfine, naftifine, butenafine, anidulafungin, caspofungin, micafungin, ciclopirox, flucytosine, griseofulvin, Gentian violet, haloprogin, tolnaftate, undecylenic acid, and combinations thereof.

### BRIEF DESCRIPTION OF THE DRAWINGS

FIG. 1 shows the β-catenin downstream proliferation (left arm) and differentiation pathway (right arm). Note that ICG-001 mediated inhibition of CBP β-catenin interaction switches on the p300/β-catenin mediated differentiation pathway.

FIG. 2 shows, according to particular exemplary aspects, confocal microscopy demonstrating GFP expression in Lgr5-GFP transgenic mice. Note surviving clonogens of Lgr5+ crypt base columnar cells (ISC) at 24 hr (A) but not at 3.5 days (B) following 18 Gy abdominal irradiation (AIR) (C) 3.5 days post-AIR and stromal cell transplantation and (D) Untreated PBS controls.

FIG. 3 shows, according to particular exemplary aspects, that systemic administration of ICG-001 plus AdRspo1, post-radiation exposure, mitigates RIGS. Treatment with AdRspo-1 followed by ICG-001, 72 hrs after irradiation, mitigates radiation lethality and rescued 70% mice (P<0.007) irradiated with 9.4 Gy and 60% mice (p<0.003) exposed to 10.4 Gy. All the untreated control mice died within 10-15 days after exposure to 9.4-10.4 Gy WBI.

FIG. 4 shows, according to particular exemplary aspects, a histopathological assessment of intestine after 10.4 Gy whole body irradiation. Histopathological evaluation of jejunum demonstrated larger crypt depth, intact villi (Hematoxylin-Eosin, HE), increased BrdU uptake in crypt and reduced apoptosis (TUNNEL) in AdRspo-1+ICG-001-treated animals, compared to irradiated controls (WBI), indicating structural regeneration of the irradiated intestine.

FIG. 5 shows that small molecule inducers of Rspo1 (e.g., arylhydrdocarbon receptor (AHR) agonists such as beta napthoflavone, which induce the expression of Rspo1) or direct Wnt/catenin activators such as LiCl, or GSK3-beta inhibitors such as CHIR, can be used to replace (for at least up to 4 days) Rspo1 for maintenance of small intestinal crypt organoid cultures ex vivo. N.B. in FIG. 5 means half dose of Rspo1.

FIG. 6 shows, according to particular exemplary aspects, a schematic of a Daedalus lenti-viral based R-spo1 expression cassette that was used to express and prepare human recombinant R-spondin1 (R-spo1).

FIG. 7 shows, according to particular exemplary aspects, FLOW cytometry analysis of Lenti-R-spo 1 transduction of 293F cells histogram shows successful (>95%) transduction of 293F cells using the R-spo 1 virus.

FIG. 8, shows, according to particular exemplary aspects, FLOW cytometry analysis of Lenti-R-spo 1 sorted population; histogram shows that the sorted population is approximately 100% GFP positive and has an almost 3-fold increase in GFP mean fluorescence intensity as compared to the pre-sort population in FIG. 7.

FIG. 9 shows, according to particular exemplary aspects, the results of running heparin purification R-spo 1 fractions on an SDS gel.

FIG. 10 shows, according to particular exemplary aspects, a UV trace of R-spo 1 size exclusion purification; figure insert shows SDS PAGE gel analysis of fractions corresponding to the aggregate peaks and R-spo 1.

FIG. 11 shows, according to particular exemplary aspects, a Top Flash Luciferase assay demonstrating that recombinant R-spo 1 activates the Wnt pathway in 293 cells.

FIGS. 12A and 12B show, according to particular exemplary aspects, testing of functionality of recombinant human Rspo1 in an intestinal organoid growth/maintenance assay. The recombinant human protein was capable of maintaining the growth and proliferation of mouse intestinal organoids in culture.

FIGS. 13A and 13B show, according to particular exemplary aspects, that treatment with hRspo1+ICG-001 mitigated radiation lethality and rescued 80% (P<0.001) of mice exposed to 10.4 Gy whole body irradiation (WBI). Mice receiving only supportive care died within 10-15 days after exposure to 10.4 Gy WBI.

### DETAILED DESCRIPTION

### Definitions

The term “CBP protein” refers to the protein that is also known as CREB-binding protein, where CREB is an abbreviation for “cAMP-response element binding”. This protein is well known in the art, see, e.g., Takemaru et al., J. Cell Biol. 149:249-54 (2000) and U.S. Pat. No. 6,063,583. CBP 1-111 refers to the first 111 amino acids of the protein CBP, as identified from the N-terminus of CBP.

The term “p300 protein” refers to a protein that is well known in the art. See, e.g., Gusterson, R. J. et al., J. Biol. Chem. 2003 Feb. 28; 278(9):6838-47; An and Roeder, J. Biol. Chem. 2003 Jan. 17; 278(3):1504-10; Rebel, V. I. et al., Proc Natl Acad Sci USA. 2002 Nov. 12; 99(23):14789-94; and U.S. Pat. No. 5,658,784, as well as references cited therein. p300 1-111 refers to the first 111 amino acids of the protein p300, as identified from the N-terminus of p300.

The phrase “Wnt pathway” refers to a signaling cascade that may be initiated by the binding of Wnt proteins (secreted glycoproteins) to frizzled seven-transmembrane-span receptors. This pathway is known and characterized in the art and is the subject of numerous articles and reviews (see, e.g., Huelsken and Behrens, J. Cell Sci. 115: 3977-8, 2002; Wodarz et al., Annu. Rev. Cell Dev. Biol. 14:59-88 (1998); Morin, P. J., Bioessays 21:1021-30 (1999); Moon et al., Science 296:1644-46 (2002); Oving et al., Eur. J. Clin. Invest 32:448-57 (2002); Sakanaka et al., Recent Prog. Horm. Res. 55: 225-36, 2000).

The phrase “the activity of the Wnt pathway” refers to the activity of at least one component of the pathway. For example, the activity of the Wnt pathway, in certain embodiments, may refer to the activity of β-catenin in inducing expression of targeted genes. Many components of the Wnt pathway are known in the art, and include but are not limited to Cerberus (Cer), Dickkopf (DKK), LRP, Frizzled heterotrimeric Gproteins, Dsh, casein kinease 1a (CK1a), GSK3β, βTrCP, ACP, Axin, CBP, p300, β-catenin, TCF, Groucho, etc.

A compound that “activates the Wnt pathway” refers to a compound that leads to β-catenin induced expression of target genes when present in a system having the Wnt pathway. Many target genes whose expression is induced by β-catenin are known in the art, and include but are not limited to Conductin, Myc, Twin, Cyclin D1, Nkd, Ubx, En-2, PPARd, Xbra, ID2, Siamois, Xnr3, MMPI, TCF-1, survivin, etc. Such genes may also be referred to as “genes targeted by the Wnt/β-catenin pathway.”

In particular aspects a “Wnt agonist” is an agent sufficient to stimulate amplification of a somatic stem cell pool, and may include, but is not limited to R-spondin1 (R-spo1), small molecule inducers of Rspo1, direct Wnt/catenin activators, LiCl, GSK3beta inhibitors, CHIR (e.g., CHIR-911), arylhydrdocarbon receptor (AHR) agonists, beta napthoflavone, indole-3-carbinol (I3C), high-affinity AhR ligands DIM and ICZ), formylindolo[3,2-b]carbazoles, 6-formylindolo[3,2-b]carbazole (FICZ) FICZ-derived indolo[3,2-b]carbazole-6-carboxylic acid metabolites and sulfoconjugates, which induce the expression of Rspo1.

The phrase “selectively inhibiting expression of genes targeted by the Wnt/β-catenin pathway” refers to inhibiting the expression of a subset of genes targeted by the Wnt/β-catenin pathway, but not inhibiting the expression of the other genes targeted by the Wnt/β-catenin pathway. Although not wished to be bound to any particular mechanism, the inventors of the present invention speculate that the selective inhibition of gene expression may be accomplished by interrupting the interaction between β-catenin and some, but not all, of its potential binding partners.

The exemplary compound ICG-001 provides an exemplary method to specifically and selectively block only the very amino terminus of CBP, which is responsible for the interaction between CBP and catenin. As the region that ICG-001 binds to on CBP is limited to the very amino terminus, it follows that the downstream changes that this compound effects are not global, but limited only to functions that this region of CBP controls.

Nuclear accidents and terrorism present a serious threat for mass casualty. With increasing doses of radiation exposure, radiation lethality is seen among victims from injury to organs, such as, bone marrow gastrointestinal tract and lung. With larger irradiation doses (≧10 Gy), victims suffer irreversible hematopoietic and gastrointestinal injury and usually perish despite supportive care and bone marrow transplantation (BMT). Currently there are no approved medical countermeasures to alleviate radiation-induced gastrointestinal syndrome (RIGS), resulting from direct cytocidal effects on intestinal stem cells (ISC) and crypt stromal cells.

Several growth factors and cytokines including KGF, TGFβ, TNFα, PGE2, and IL11 have been shown to protect intestine from radiation or other cytotoxic injury by increasing the crypt cell proliferation and survival (1-4). Applicants have recently demonstrated that R-spondin1 (R-spo1) played a protective role in RIGS by amplifying the intestinal stem cell (ISC) population. However, such growth factors, although highly effective in protecting and mitigating RIGS in lower doses, fail to effectively mitigate after whole body exposure of doses≧10 Gy. Applicants have additionally demonstrated that intravenous transplantation of bone marrow-derived stromal cells, 24 hours after exposure to 10.4-12 Gy of whole body irradiation or 18-20 Gy of total abdominal irradiation, mitigated RIGS and rescued animals from radiation lethality. Interestingly, serum levels of R-spo1, KGF, PDGF and bFGF were elevated in the irradiated and transplanted animals. We also noted that the Lgr5+ve, crypt columnar basal cells (CBC), the putative ISCs, were maintained up to 24-30 hrs following irradiation, providing us a window of mitigation for repair and regeneration of these irradiated ISCs.

Survival of animals from RIGS depends upon the fate of the crypt ISCs and is determined by clonogenic cell repair capabilities of the ISC. If all crypt cells die, the crypt is sterilized and disappears within 48 hours. However if one or more ‘clonogenic cell’ survives the insult, it rapidly proliferates to regenerate the crypt within 72-96 hours and subsequently tissue heals by clonal expansion. The survival of the animal depends on the rate of the crypt depopulation and the efficiency and number of the surviving clonogenic cells capable of generating a crypt. The Wnt-β-catenin/T cell factor (TCF) signal transduction pathway plays a critical role in the regulation of proliferation and differentiation of the intestinal epithelium, as it undergoes rapid and continuous self-renewal along the crypt-villus axis (5-8). The R-spondin (roof plate-specific spondin) protein family comprises a novel family of secreted proteins, which act as major agonists and modulators of the Wnt-β-catenin signaling pathway (9, 10). Of these, the human Rspo1 with a molecular weight of 29 kd (263 amino acids) has a specific proliferating effect on intestinal crypt cells (11) and is highly effective in inducing repair and stimulating proliferation of the irradiated ISCs. The Wnt proteins are central to the maintenance of the undifferentiated state of crypt ISCs (5-8, 12). Upon differentiation of ISCs to enterocytes along the villi the levels of Wnt protein decrease, while bone morphogenic protein (BMP) levels increase. Thus inhibition of Wnt is critical to accelerate differentiation of ISCs for villous regeneration post-radiation exposure.

Applicants have now further conceived that an ideal therapy for RIGS would comprise a multimodal therapy/treatment that stimulates ISC regeneration, restores the ISC niche and accelerates differentiation of the ISCs with maintenance of the GI mucosal integrity.

Applicants, therefore, specifically hypothesized that a sequential administration of a Wnt agonist (e.g., Rspo1) that could amplify the surviving ISC pool post-radiation exposure, followed by a selective antagonist of the β-Catenin-CBP interaction (e.g., ICG-001) that could accelerate differentiation of Rspo1-stimulated ISC in crypt-villi axis, would promote villous regeneration and intestinal restitution, thereby, mitigating RIGS. Applicants conceived that the acute loss of stem/progenitors and stromal cells in the stem cell niche following radiation-induced damage would require rapid compensation of their functions by inducing proliferation of surviving clonogens followed by differentiation.

Wnt/β-catenin signaling has been demonstrated to maintain pluripotency in stem cells under certain conditions, but is also critical for the expansion of progenitors. Applicants have recently developed a model to explain these divergent responses to activation of Wnt/β-catenin signaling. The model posits that β-catenin/CBP-mediated transcription is critical for cell proliferation without differentiation, whereas β-catenin/p300-mediated transcription is critical for commitment to a differentiative program with a more limited proliferative capacity.

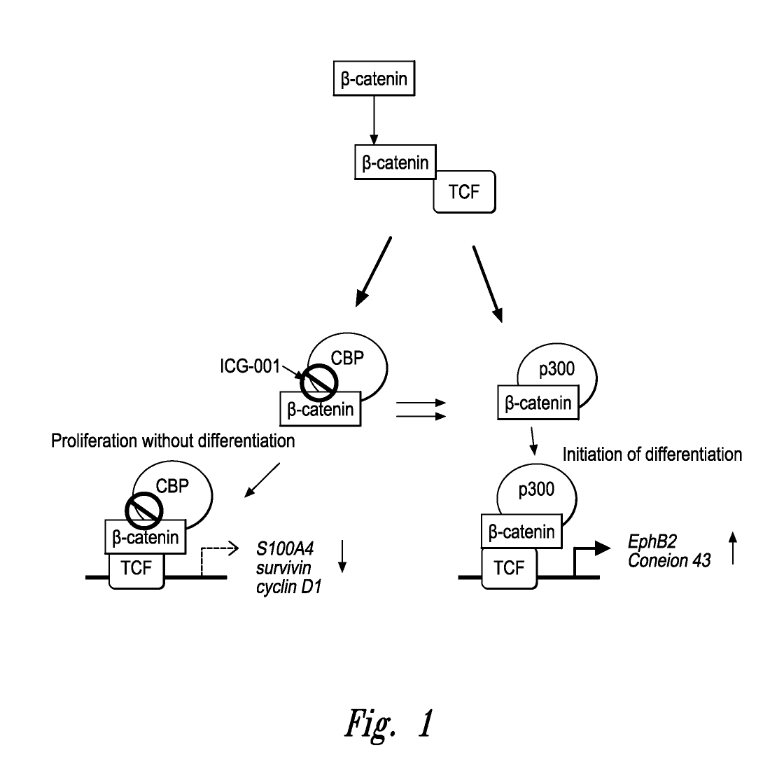

The homeostasis of intestinal epithelium is normally maintained by an intricate cell replacement process in which terminally differentiated epithelial cells are continuously and rapidly replaced by replicated undifferentiated epithelial cells (transit cells) located within the crypts of Lieberkuhn. The pluripotent stem cell that resides near the crypt base gives rise to progenitors that proliferate by means of Wnt signaling and the activation of β-catenin. Normally, the Wnt proteins provide a prototype for the ligand-mediated activation signaling, and their activities in proliferation involve a decreasing gradient from stem cells to the upper crypt regions. With decreased Wnt signaling (e.g., upper crypt regions), b-catenin forms a complex with APC and axin (destruction complex), leading to the degradation and lower b-catenin levels (13). Applicants were the first to demonstrate that human Rspo1 can act as an intestinal stem cell growth factor, which stimulates the proliferation of Lgr5+ve, ISCs and protect mice from RIGS. β-catenin activation is dependent on the binding of Wnt to both Frizzled (FZD) and the LRP6 co-receptor. Binding of LRP6 by Kremen supports DDK1-mediated internalization of LRP6 and the associated degradation of β-catenin, resulting in the cessation of signaling pathways required for crypt cell regeneration. Disruption of the LRP6:Kremen interaction thus represents a significant therapeutic target for the promotion of crypt cell regeneration. Notably, R-spondin acts as a major activator of Wnt-β-catenin signaling by directly competing with Kremen for binding to LRP6. Once, the Rspo1 (the receptor for Rspo seems to be lgr5 in crypts. Lgr5 is a GPCR) binds to LRP6 with subsequent phosphorylation of Dishevelled, the protein complex containing axin, GSK and APC is deactivated and fail to initiate proteolytic degradation of beta-catenin. Beta-catenin translocates to the nucleus and forms a complex with T-cell transcription factor/lymphoid enhancer binding factor (TCF/LEF). At this point, there are two transcriptional co-activators that determine the transcription signaling by interacting with the b-catenin/TCF complex: the CREB binding protein (CBP) that induces proliferation without differentiation or the highly homologous p300 protein that initiates differentiation. The TCF/β-catenin/CBP-mediated transcription is critical for proliferation without differentiation (e.g., in cancer and stem cells) (FIG. 1, left arm of the pathway). However, the switch of co-activators by the TCF/β-catenin complex is the essential first step in the initiation of differentiation. The TCF/β-catenin/p300 complex then drives the transcription of the Wnt/β-catenin regulated genes associated with normal cellular differentiation (FIG. 1, right arm) (14). Aberrant regulation of the balance between these two related transcriptional programs will dysregulate the intestinal homeostasis.

FIG. 1 shows β-catenin downstream proliferation (left arm) and differentiation pathway (right arm). Note that ICG-001 mediated inhibition of CBP β-catenin interaction switches on p300 TCF mediated differentiation pathway.

ICG-001 is a small molecule secondary structure peptidomimetic that disrupts the interaction of the CBP and b-catenin, thereby, inhibiting Wnt-mediated transcription of genes responsible for proliferation (14, 15). The primary mechanism of action of ICG-001 is to antagonize β-catenin/TCF mediated transcription via binding to CAMP response element binding protein (CBP). Administration of ICG-001 would be expected to inhibit binding of β-catenin/TCF with CBP, thereby augmenting the binding of p300 to β-catenin/TCF complex and switch the signaling from stem cell proliferation to differentiation.

According to particular aspects of the present invention, Applicants hypothesized that since Wnt levels are reduced as crypt transit cells differentiate in the crypt-villi axes, a CBP/β-catenin inhibitor (e.g., ICG-001) would antagonize Wnt-mediated proliferation of ISC and accelerate the differentiation of the immature daughter cells arising from Rspo1-stimulated ISCs.

According to particular aspects of the present invention, the method depends upon the sequential administration of a Wnt pathway activator (e.g., R-spondin1 (R-spo1), direct Wnt/catenin activators, LiCl, GSK3-beta inhibitors, CHIR (e.g., CHIR-911), small molecule inducers of Rspo1, arylhydrdocarbon receptor (AHR) agonists, beta napthoflavone, indole-3-carbinol (I3C), high-affinity AhR ligands DIM and ICZ), formylindolo[3,2-b]carbazols, 6-formylindolo[3,2-b]carbazole (FICZ) FICZ-derived indolo[3,2-b]carbazole-6-carboxylic acid metabolites and sulfoconjugates, which induce the expression of Rspo1), that would amplify the surviving ISC pool post-radiation exposure, followed by administration of an intestinal differentiation agent (e.g., ICG-001), 2-4 days after radiation exposure to accelerate differentiation of Rspo1-stimulated progenitor and transit cells in crypt-villi axis, and to augment villous regeneration and intestinal restitution, thereby, mitigating RIGS.

According to additional aspects, supportive therapy is provided to prevent infection and dehydration.

According to additional aspects, while growth factors, such as, KGF, IL-11, PDGF can protect and increase crypt cell proliferation they cannot accelerate differentiation and therefore, are mostly ineffective in mitigating RIGS.

According to particular aspects, a stem cell differentiation agent (e.g., ICG-001) was used to augment differentiation of R-spo1-amplified ISCs and mitigate RIGS in irradiated subjects (e.g., C57Bl/6 mice).

In particular aspects, the results disclosed herein demonstrated that administration of ICG-001, 72 hours after irradiation, rescued 60% of mice exposed to 10.4 Gy whole body irradiation. According to particular aspects, this is the first differentiation agent that has been combined with multiple growth factors for successful mitigation of RIGS.

According to particular aspects, sequential administration of a recombinant adenovirus expressing R-spondin1 (within 24 hrs post exposure) with ICG-001 (72 hrs post-exposure), a CBP/catenin antagonist, mitigated radiation injury and improved survival in mice that were exposed to lethal doses of whole body irradiation (10.4 Gy in single fraction), thus providing the first demonstration to Applicants' knowledge of mitigation for acute radiation injury with small molecular Wnt modulators.

According to particular aspects, the disclosed methods have substantial utility for protection (e.g., administered before exposure to radiation to prevent radiation injury), and/or mitigation (e.g., administered after radiation exposure but before induction of clinical symptoms from radiation injury), and/or treatment of symptoms (e.g., administered after radiation exposure and appearance of clinical symptoms of radiation injury) of radiation injury.

According to particular aspects, the disclosed methods have substantial utility for treating victims of nuclear accidents and nuclear terrorism.

Additional aspects provide methods for treating first responders and nuclear power workers responding to nuclear accidents.

Additional aspects provide methods for treating military personnel with potential nuclear warfare.

Additional aspects provide methods for treating radiation injury in cancer patients that received or will receive radiation therapy.

Additional aspects provide methods for treating radiation-induced gastrointestinal syndrome (RIGS).

Additional aspects provide methods for treating radiation-induced pulmonary syndrome (RIPS).

Additional aspects provide methods for treating radiation induced bone marrow syndrome (RIBMS).

Additional aspects provide methods for treating radiation-induced bladder injury.

Additional aspects provide methods for treating radiation-induced liver damage (RILD).

Additional aspects provide methods for treating radiation-induced salivary gland injury.

Additional aspects provide methods for treating radiation-induced kidney injury.

Additional aspects provide methods for treating acute or chronic radiation proctitis.

Additional aspects provide methods for treating radiation esophagitis.

Additional aspects provide methods for treating radiation-induced cutaneous ulcer and fibrosis.

Additional aspects provide methods for treating radiation-induced pharyngeal fibrosis and dysfunction.

Additional aspects provide methods for treating chronic radiation-induced mucosal ulcers and fistulae.

Further aspects provide for protective treatment for cancer patients undergoing high dose radiation therapy and chemotherapy.

Further aspects provide for treatment of chemotherapy-induced mucositis.

In particular embodiments of the methods, the CBP/catenin (e.g., CBP/β-catenin) antagonist is at least one selected from the group of compounds and salts thereof encompassed by Table 1 or otherwise disclosed herein. In certain embodiments, the CBP/β-catenin antagonist comprises ICG-001.

Certain aspects of methods comprise co-administration of or adjunct treatment with at least one other therapeutic agent (e.g., such as simultaneously or adjunctively treating the subject with an anti-inflammatory agent). In certain aspects, the anti-inflammatory agent comprises a steroid or glucocorticoid steroid. In particular embodiments, the at least one anti-inflammatory agent is selected from the group consisting of: short-acting β₂-agonists, long-acting β₂-agonists, anticholinergics, corticosteroids, systemic corticosteroids, mast cell stabilizers, leukotriene modifiers, methylxanthines, β₂-agonists, albuterol, levalbuterol, pirbuterol, artformoterol, formoterol, salmeterol, anticholinergics including ipratropium and tiotropium; corticosteroids including beclomethasone, budesonide, flunisolide, fluticasone, mometasone, triamcinolone, methyprednisolone, prednisolone, prednisone; leukotriene modifiers including montelukast, zafirlukast, and zileuton; mast cell stabilizers including cromolyn and nedocromil; methylxanthines including theophylline; combination drugs including ipratropium and albuterol, fluticasone and salmeterol, glucocorticoid steroids, budesonide and formoterol; antihistamines including hydroxyzine, diphenhydramine, loratadine, cetirizine, and hydrocortisone; immune system modulating drugs including tacrolimus and pimecrolimus; cyclosporine; azathioprine; mycophenolatemofetil; and combinations thereof.

Antiviral (RSV) combinations may include a nucleoside analog (e.g., acyclovir or docosanol (active ingredient in Abreva)).

In certain aspects, the one additional therapeutic agent is selected from the group consisting of anti-microbial agents, antifungal agents, and antibiotic agents. In particular embodiments, the at least one additional therapeutic agent is selected from the group consisting of: ciclosporin, hyaluronic acid, carmellose, macrogol(s), dextran and hyprolose, sodium and calcium, sodium and povidone, hypromellose, carbomer, amikacin, gentamicin, kanamycin, neomycin, netilmicin, streptomycin, tobramycin, paromomycin, geldanamycin, herimycin, loracarbef, ertapenem, imipenem/cilastatin, meropenem, cefadroxil, cefazolin, cefalotin/cefalothin, cephalexin, cefaclor, cefamandole, cefoxitin, cefuroxime, cefixime, cefdinir, cefditoren, cefoperazone, cefotaxime, cefpodoxime, ceftazidime, ceftibuten, ceftizoxime, ceftriaxone, cefeprime, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, nafcillin, penicillin, peperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, protosil, sulfacetamide, sulfamethizole, sulfanilamide, sulfasalazine, sulfisoxazole, trimethoprim, trimethoprim-sulfamethoxazole, demeclocycline, doxycycline, minocycline, oxytetracycline, tetracycline, arsphenamine, chloramphenicol, clindamycin, lincoamycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin/rifampicin, tinidazole, miconazole, ketoconazole, clotrimazole, econazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, teronazole, terbinafine, amorolfine, naftifine, butenafine, anidulafungin, caspofungin, micafungin, ciclopirox, flucytosine, griseofulvin, Gentian violet, haloprogin, tolnaftate, undecylenic acid, and combinations thereof.

### Methods of Treatment

The term “treating” refers to, and includes, reversing, alleviating, inhibiting the progress of, or preventing a disease, disorder or condition, or one or more symptoms thereof; and “treatment” and “therapeutically” refer to the act of treating, as defined herein.

A “therapeutically effective amount” is any amount of any of the compounds utilized in the course of practicing the invention provided herein that is sufficient to reverse, alleviate, inhibit the progress of, or prevent a disease, disorder or condition, or one or more symptoms thereof.

The therapeutic compositions of the present invention include compositions that are able to be administered to a subject in need thereof. As used herein, “subject,” may refer to any living creature, preferably an animal, more preferably a mammal, and even more preferably a human.

In certain embodiments, the composition formulation may also comprise at least one additional agent selected from the group consisting of: carriers, adjuvants, emulsifying agents, suspending agents, sweeteners, flavorings, perfumes, and binding agents.

As used herein, “pharmaceutically acceptable carrier” and “carrier” generally refer to a non-toxic, inert solid, semi-solid or liquid filler, diluent, encapsulating material or formulation auxiliary of any type (e.g., including creams and lotions, emulsions, jellies, depot formulations). Some non-limiting examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutically acceptable carriers described herein, for example, vehicles, adjuvants, excipients, or diluents, are well-known to those who are skilled in the art. Typically, the pharmaceutically acceptable carrier is chemically inert to the therapeutic agents and has no detrimental side effects or toxicity under the conditions of use. The pharmaceutically acceptable carriers can include polymers and polymer matrices, nanoparticles, microbubbles, and the like.

In addition to the therapeutic CBP/catenin (e.g., CBP/β-catenin) antagonists of the present invention, the therapeutic composition may further comprise inert diluents such as additional solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

### Administrative Routes

Most suitable means of administration for a particular subject will depend on the nature and severity of the disease or condition being treated or the nature of the therapy being used, as well as the nature of the therapeutic composition or additional therapeutic agent. In certain embodiments, oral or intra venus (i.v.) is preferred. In some embodiments, sub cutaneous (c.p.), intra peritoneal (i.p.), or topical is used. Administration of Rspo1 protein is preferably via i.v., i.p., or s.c.

Formulations suitable for oral administration may be provided as discrete units, such as tablets, capsules, cachets, syrups, elixirs, chewing gum, “lollipop” formulations, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

### Exemplary CBP/Catenin (e.g., CBP/β-Catenin) Antagonists Having Utility in the Herein Disclosed Methods

In particular embodiments of the herein described methods, the CBP/catenin (e.g., CBP/β-catenin) antagonist is at least one selected from the group of compounds and salts thereof encompassed by Table 1 or otherwise disclosed herein. In certain aspects, the CBP/catenin (e.g., CBP/β-catenin) antagonist comprises ICG-001 or an active derivative thereof.

Preferably, administration of the CBP/catenin (e.g., CBP/β-catenin) antagonist comprises oral and/or IV administration, and/or topical.

Certain aspects of methods comprise co-administration of or adjunct treatment with at least one other therapeutic agent (e.g., such as simultaneously or adjunctively treating the subject with an anti-inflammatory agent). In certain aspects, the anti-inflammatory agent comprises a steroid or glucocorticoid steroid. In particular embodiments, the at least one anti-inflammatory agent is selected from the group consisting of: short-acting β₂-agonists, long-acting β₂-agonists, anticholinergics, corticosteroids, systemic corticosteroids, mast cell stabilizers, leukotriene modifiers, methylxanthines, β₂-agonists, albuterol, levalbuterol, pirbuterol, artformoterol, formoterol, salmeterol, anticholinergics including ipratropium and tiotropium; corticosteroids including beclomethasone, budesonide, flunisolide, fluticasone, mometasone, triamcinolone, methyprednisolone, prednisolone, prednisone; leukotriene modifiers including montelukast, zafirlukast, and zileuton; mast cell stabilizers including cromolyn and nedocromil; methylxanthines including theophylline; combination drugs including ipratropium and albuterol, fluticasone and salmeterol, glucocorticoid steroids, budesonide and formoterol; antihistamines including hydroxyzine, diphenhydramine, loratadine, cetirizine, and hydrocortisone; immune system modulating drugs including tacrolimus and pimecrolimus; cyclosporine; azathioprine; mycophenolatemofetil; and combinations thereof.

In certain aspects, the one additional therapeutic agent is selected from the group consisting of anti-microbial agents, antifungal agents, and antibiotic agents. In particular embodiments, the at least one additional therapeutic agent is selected from the group consisting of: ciclosporin, hyaluronic acid, carmellose, macrogol(s), dextran and hyprolose, sodium and calcium, sodium and povidone, hypromellose, carbomer, amikacin, gentamicin, kanamycin, neomycin, netilmicin, streptomycin, tobramycin, paromomycin, geldanamycin, herimycin, loracarbef, ertapenem, imipenem/cilastatin, meropenem, cefadroxil, cefazolin, cefalotin/cefalothin, cephalexin, cefaclor, cefamandole, cefoxitin, cefuroxime, cefixime, cefdinir, cefditoren, cefoperazone, cefotaxime, cefpodoxime, ceftazidime, ceftibuten, ceftizoxime, ceftriaxone, cefeprime, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, nafcillin, penicillin, peperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, protosil, sulfacetamide, sulfamethizole, sulfanilamide, sulfasalazine, sulfisoxazole, trimethoprim, trimethoprim-sulfamethoxazole, demeclocycline, doxycycline, minocycline, oxytetracycline, tetracycline, arsphenamine, chloramphenicol, clindamycin, lincoamycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin/rifampicin, tinidazole, miconazole, ketoconazole, clotrimazole, econazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, teronazole, terbinafine, amorolfine, naftifine, butenafine, anidulafungin, caspofungin, micafungin, ciclopirox, flucytosine, griseofulvin, Gentian violet, haloprogin, tolnaftate, undecylenic acid, and combinations thereof.

Particular aspects provide formulations for administration of CBP/catenin (e.g., CBP/β-catenin) antagonists, such as those of TABLE 1.

TABLE 1
Exemplary CBP/β-catenin antagonists having utility for methods disclosed herein.
All compound genera, species and conformations thereof, and syntheses thereof, of the patent
applications and patents of this Table are incorporated by reference herein in their entireties, as
exemplary compounds having utility for the presently claimed methods.

| Publication/Patent | Publication/Issuance | Compound | |
|---|---|---|---|
| No. | date | class | Compound genus |
| US 2005/0250780 | 10 Nov. 2005; MMX | Reverse turn mimetics; All genera and compounds and salts thereof disclosed and claimed therein | 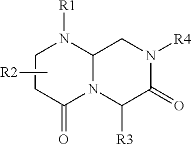 |
| US 2007/0021431 A1 | 25 Jan. 2007; CWP | Reverse turn mimetics; All genera and compounds and salts thereof disclosed and claimed therein | 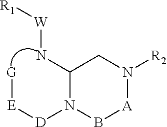 |
| US 2007/0021425 A1 | 25 Jan. 2007; CWP | Reverse turn mimetics; All genera and compounds and salts thereof disclosed and claimed therein | 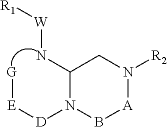 |
| US 2010/0120758 A1 | 13 May 2010 | Reverse turn mimetics; All genera and compounds and salts thereof disclosed and claimed therein | 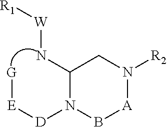 |
| US 2010/0240662 A1 | 23 Sep. 2010 | Reverse turn mimetics; All genera and compounds and salts thereof disclosed and claimed therein | 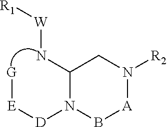 |
| WO 2007/139346 A1 | 6 Dec. 2007 | Reverse turn mimetics; All genera and compounds and salts thereof disclosed and claimed therein | 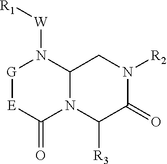 |
| US 2004/0072831 A1 | 15 Apr. 2004 | Reverse turn mimetics; All genera and compounds and salts thereof disclosed and claimed therein | 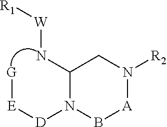 |
| US 2007/0043052 | 22 Feb. 2007 | Reverse turn mimetics; All genera and compounds and salts thereof disclosed and claimed therein | 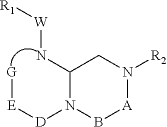 |
| US 2005/0059628 A1 | 17 Mar. 2005 | Reverse turn mimetics; All genera and compounds and salts thereof disclosed and claimed therein | 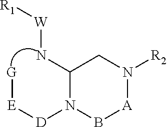 |
| WO 2009/051399 A2 | 23 Apr. 2009 | Reverse turn mimetics; All genera and compounds and salts thereof disclosed and claimed therein | 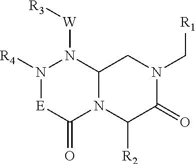 |
| US 2006/0084655 A1 | 20 Apr. 2006 | Reverse turn mimetics; All genera and compounds and salts thereof disclosed and claimed therein | 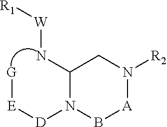 |
| US 2008/0009500 A1 | 10 Jan. 2008 | Reverse turn mimetics; All genera and compounds and salts thereof disclosed and claimed therein | 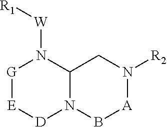 |
| US 2010/0222303 | 2 Sep. 2010 | Reverse turn mimetics; All genera and compounds and salts thereof disclosed and claimed therein | 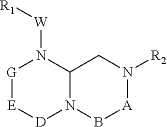 |
| U.S. Pat. No. 6,413,963 | Issued 2 Jul. 2002; MMX | Reverse turn mimetics; All genera and compounds and salts thereof disclosed and claimed therein | 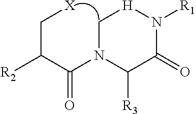 |
| U.S. Pat. No. 7,531,320 B2 | 12 May 2009 | Reverse turn mimetics; All genera and compounds and salts thereof disclosed and claimed therein | 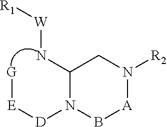 |
| U.S. Pat. No. 7,563,825 | 21 Jul. 2009 | Reverse turn mimetics; All genera and compounds and salts thereof disclosed and claimed therein | 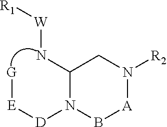 |

### Exemplary Compound Genera (Cont.)

US 2005/0250780. All compound genera, species and conformations thereof of US 005/0250780, including the exemplary compounds of Tables 2-6 thereof, the claimed compounds, and including the disclosed respective syntheses, are incorporated herein by reference in their entirety as exemplary compounds for use in applicant's presently claimed methods.

Specific exemplary embodiments include a compound having the structure (I):

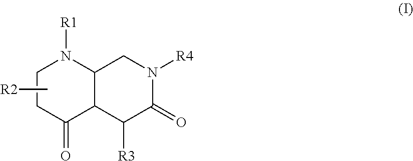

or a pharmaceutically acceptable salt or stereoisomer thereof,
wherein
R1 is —X—R5, where X is —C(═O)—, —C(═O)O—, —C(═O)NH— or —SO2-, and R5 is an amino acid side chain moiety or amino acid side chain derivative;
R2 is hydrogen or —Y—R6, where Y is a direct bond, —NH—, —NHC(═O)—, —NHC(═O)O—, —NHC(═O)NH— or —NHSO2-, and R6 is an amino acid side chain moiety or amino acid side chain derivative;
R3 is —Z—R7, where Z is a direct bond, —(CH2)mC(═O)NR8-, —(CH2)kNHC(═O) or —(CH2)kNHC(═O)NR8-, R7 and R8 are independently amino acid side chain moieties or amino acid side chain derivatives, m is an integer from 1 to 4 and k is 1 or 2;
R4 represents the remainder of the compound; and
wherein any two adjacent CH groups or adjacent NH and CH groups of the fused bicyclic compound optionally form a double bond.

Additional specific exemplary embodiments include those compounds of structure (I) wherein X is —C(C-0)0-, R2 is H, C1-C6 alkyl, or C7-C11 arylalkyl; R3 is —(CH₂)₁₋₆—N(R′)(R″), wherein R′ and R″ are independently H or —C(NH)(NH2); R4 is C7-C11 arylalkyl; and R5 is C7-C11 arylalkyl, and wherein R4 and R5 are optionally and independently substituted with 1-3 halogen, 1-3 C1-C3 haloalkyl, or 1-3 C1-C3 alkyl.

Further specific exemplary embodiments the compounds include those compounds of structure

(I), wherein X is —C(C—O)NH—, R2 is H, C1-C6 alkyl, or C7-C11 arylalkyl; R3 is

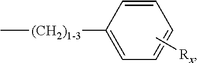

wherein Rx is H, OH or halo; R4 is C7-C11 arylalkyl; and R5 is C7-C11 arylalkyl, and wherein R2, R4 and R5 are optionally and independently substituted with 1-3 halogens, 1-3 C1-C3 haloalkyls, or 1-3 C1-C3 alkyl.
US 2007/0021431. All compound genera, species and conformations thereof of US 2007/0021431, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having the structure (I):

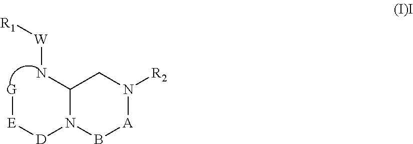

or a pharmaceutically acceptable salt or stereoisomer thereof, wherein A is —(CHR3)- or —(C═O)—, B is —(CHR4)-, —(C═O)—, D is —(CHR5)- or —(C═O)—, E is —(ZR6)-, —(C═O)—, G is —(XR7)n-, —(CHR7)-(NR8)-, —(C═O)—(XR9)-, or —(C═O)—, W is —Y(C═O)—, —(C═O)NH—, —(SO2)- or nothing, Y is oxygen, sulfur or —NH—, X and Z is independently nitrogen or CH, n=0 or 1; and R1, R2, R3, R4, R5, R6, R7, R8 and R9 are the same or different and independently selected from an amino acid side chain moiety or derivative thereof, the remainder of the molecule, a linker and a solid support, and stereoisomers thereof, and as defined in US 2007/0021431.
US 2007/0021425. All compound genera, species and conformations thereof of US 2007/0021425, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having the structure following general formula (I):

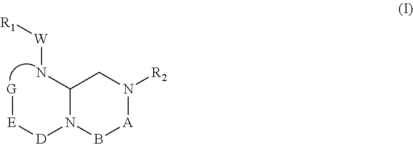

wherein A is —(CHR₃)— or —(C═O)—, B is —(CHR₄)— or —(C═O)—, D is —(CHR₅)— or —(C═O)—, E is —(ZR₆)— or —(C═O)—, G is —(XR₇)ₙ—, —(CHR₇)—(NR₈)—, —(C═O)—(XR₉)—, or —(C═O)—, W is —Y(C═O)—, —(C═O)NH—, —(SO₂)— or is absent, Y is oxygen, sulfur, or —NH—, X and Z is independently nitrogen or CH, n=0 or 1; and R₁, R₂, R₃, R₄, R₅, R₆, R₇, R₈ and R₉ are the same or different and independently selected from an amino acid side chain moiety or derivative thereof, the remainder of the molecule, a linker and a solid support, and stereoisomers thereof, and as defined in US 2007/0021425.

In exemplary embodiments wherein A is —(CHR₃)—, B is —(C═O)—, D is —(CHR₅)—, E is —(C═O)—, and G is —(XR₇)ₙ—, the compounds of this invention have the following formula (II):

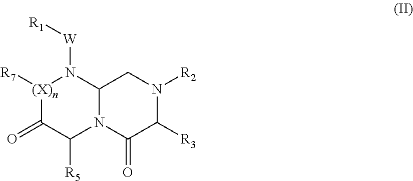

wherein W, X, Y and n are as defined above, and R₁, R₂, R₃, R₅ and R₇ are as defined in US 2007/0021425.

In exemplary embodiments wherein A is —(C═O)—, B is —(CHR₄)—, D is —(C═O)—, E is —(ZR₆)—, and G is —(C═O)—(XR₉)—, the compounds of this invention have the following formula (III):

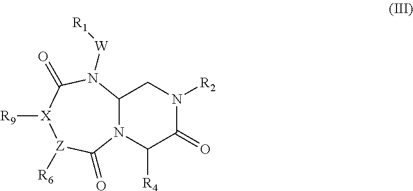

wherein W, X and Y are as defined above, Z is nitrogen or CH (with the proviso that when Z is CH, then X is nitrogen), and R₁, R₂, R₄, R₆ and R₉ are as defined in US 2007/0021425.

In exemplary embodiments wherein A is —(C═O)—, B is —(CHR₄)—, D is —(C═O)—, E is —(ZR₆)—, and G is —(XR₇)ₙ—, the compounds of this invention have the following general formula (IV):

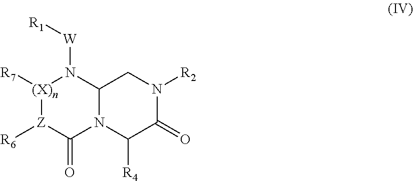

wherein W, Y and n are as defined above, Z is nitrogen or CH (when Z is nitrogen, then n is zero, and when Z is CH, then X is nitrogen and n is not zero), and R₁, R₂, R₄, R₆ and R₇, are as defined in US 2007/0021425.

In certain embodiments, the compounds of this invention have the following general formula (VI):

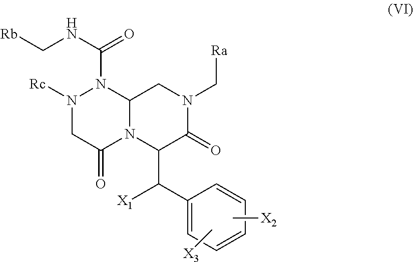

wherein Rₐ is a phenyl group; a substituted phenyl group having one or more substituents wherein the one or more substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl groups; a benzyl group; a substituted benzyl group with one or more substituents where the one or more substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; or a bicyclic aryl group having 8 to 11 ring members, which may have 1 to 3 heteroatoms selected from nitrogen, oxygen or sulfur; R_{b} is a monocyclic aryl group having 5 to 7 ring members, which may have 1 to 2 heteroatoms selected from nitrogen, oxygen or sulfur, and aryl ring in the compound may have one or more substituents selected from a group consisting of halide, hydroxy, cyano, lower alkyl, and lower alkoxy groups; R, is a saturated or unsaturated C₁₋₆alkyl, C₁₋₆alkoxy, perfluoro C₁₋₆alkyl group; and X₁, X₂, and X₃ may be the same or different and independently selected from hydrogen, hydroxyl, and halide.

The present invention is also related to prodrugs using the libraries containing one or more compounds of formula (I). A prodrug is typically designed to release the active drug in the body during or after absorption by enzymatic and/or chemical hydrolysis. The prodrug approach is an effective means of improving the oral bioavailability or i.v. administration of poorly water-soluble drugs by chemical derivatization to more water-soluble compounds. The most commonly used prodrug approach for increasing aqueous solubility of drugs containing a hydroxyl group is to produce esters containing an ionizable group; e.g., phosphate group, carboxylate group, alkylamino group (Fleisher et al., Advanced Drug Delivery Reviews, 115-130, 1996; Davis et al., Cancer Res., 7247-7253, 2002, Golik et al., Bioorg. Med. Chem. Lett., 1837-1842, 1996).

In certain embodiments, the prodrugs of the present invention have the following general formula (VII):

—Y—R₁₀ (VI)

wherein (VI) is general formula (VI) as described above; Y is oxygen, sulfur, or nitrogen of a group selected from Rₐ, R_{b}, R_{c}, X₁, X₂ and X₃;
R₁₀ is phosphate, hemisuccinate, phosphoryloxymethyloxycarbonyl, dimethylaminoacetate, amino acid, or a salt thereof; and wherein the prodrugs are capable of serving as a substrate for a phosphatase or a carboxylase and are thereby converted to compounds having general formula (VI).
US 2010/0120758. All compound genera, species and conformations thereof of US 2010/0120758, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having the structure (I):

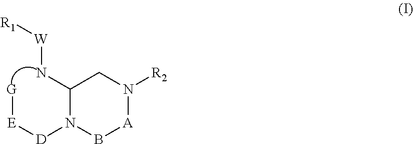

or a pharmaceutically acceptable salt or stereoisomer thereof, wherein A is —(CHR3)- or —(C═O)—, B is —(CHR4)-, —(C═O)—, D is —(CHR5)- or —(C═O)—, E is —(ZR6)-, —(C═O)—, G is —(XR7)n-, —(CHR7)-(NR8)-, —(C═O)—(XR9)-, or —(C═O)—, W is —Y(C═O)—, —(C═O)NH—, —(SO2)- or nothing, Y is oxygen, sulfur or —NH—, X and Z is independently nitrogen or CH, n=0 or 1; and R1, R2, R3, R4, R5, R6, R7, R8 and R9 are the same or different and independently selected from an amino acid side chain moiety or derivative thereof, the remainder of the molecule, a linker and a solid support, and stereoisomers thereof, and as defined in US 2010/0120758.

Specific exemplary embodiments include a compound of formula VI.

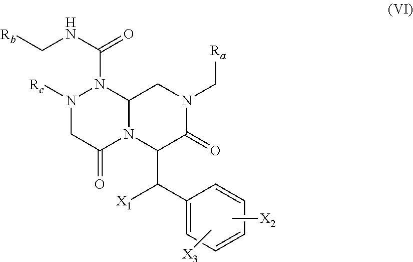

as an isolated stereoisomer or a mixture of stereoisomers or as a pharmaceutically acceptable salt, wherein,
Ra is a bicyclic aryl group having 8 to 11 ring members, which may have 1 to 3 heteroatoms selected from the group consisting of nitrogen, oxygen and sulfur;
Rb is a monocyclic aryl group having 5 to 7 ring members, which may have 1 to 2 heteroatoms selected from nitrogen, oxygen or sulfur, and aryl ring in the compound may have one or more substituents selected from a group consisting of halide, hydroxy, cyano, lower alkyl, and lower alkoxy groups;
Rc is a saturated or unsaturated C1-6alkyl, C1-6alkoxy, perfluoro C1-6alkyl group; and
X1, X2, and X3 may be the same or different and independently selected from hydrogen, hydroxyl, and halide.
US 2010/0240662. All compound genera, species and conformations thereof of US 2010/0240662, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having the structure (I):

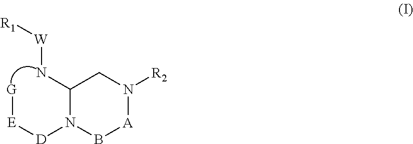

or a pharmaceutically acceptable salt or stereoisomer thereof, wherein A is —(CHR3)- or —(C═O)—, B is —(CHR4)-, —(C═O)—, D is —(CHR5)- or —(C═O)—, E is —(ZR6)-, —(C═O)—, G is —(XR7)n-, —(CHR7)-(NR8)-, —(C═O)—(XR9)-, or —(C═O)—, W is —Y(C═O)—, —(C═O)NH—, —(SO2)- or nothing, Y is oxygen, sulfur or —NH—, X and Z is independently nitrogen or CH, n=0 or 1; and R1, R2, R3, R4, R5, R6, R7, R8 and R9 are the same or different and independently selected from an amino acid side chain moiety or derivative thereof, the remainder of the molecule, a linker and a solid support, and stereoisomers thereof, and as defined in US 2010/0240662.

In exemplary embodiments wherein A is —(CHR₃)— or —(C═O)—; B is —(CHR₄)— or —(C═O)—; D is —(CHR₅)— or —(C═O)—; E is —ZR₆— or —(C═O)—, wherein Z is CH or N; G is —XR₇— or —(C═O)—, wherein X is CH or N; W is —(C═O)NH—, —(C═O)S—, —S(O)₂— or nothing; and each of R₁, R₂, R₃, R₄, R₅, R₆ and R₇ is the same or different and independently an amino acid side chain moiety or an amino acid side chain derivative, the compounds of this invention have the following formula (IA):

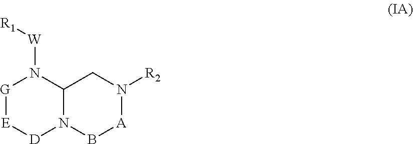

Specific examples of R₁, R₂, R₃, R₄, R₅, R₆ and R₇ are as defined in US 2010/0240662.
WO 2007/139346. All compound genera, species and conformations thereof of US 2010/0240662, including the exemplary compounds of Tables 1-2 thereof, the claimed compounds, and including the disclosed respective syntheses, are incorporated herein by reference in their entirety as exemplary compounds for use in applicant's presently claimed methods.

Specific exemplary embodiments include a compound having formula (I):

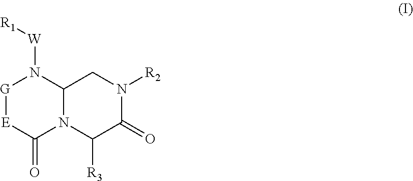

wherein:
E is —(ZR₄)— or —(C═O)—; G is nothing, —(XR₅)—, or —(C═O)—; W is —Y(C═O)—, —(C═O)NH—, —(SO₂)— or nothing; Y is oxygen or sulfur; X or Z is independently nitrogen or CH; R1, R₂, R₃, R₄, and R₅ are the same or different and independently selected from the group consisting of: an amino acid side chain moiety; C₁₋₁₂alkyl or substituted C₁₋₁₂alkyl having one or more substituents independently selected from amino, guanidino, C₁₋₄alkylguanidino, diC₁₋₄alkylguanidino, amidino, C₁₋₄alkylamidino, diC₁₋₄alkylamidino, C₁₋₅alkylamino, diC₁₋₅alkylamino, sulfide, carboxyl, hydroxyl;
C₁₋₆alkoxy;
C₆₋₁₂aryl or substituted C₆₋₁₂aryl having one or more substituents independently selected from amino, amidino, guanidino, hydrazino, C₁₋₄alkylamino, C₁₋₄dialkylamino, halogen, perfluoro C₁₋₄alkyl, C₁₋₄alkyl, C₁₋₃alkoxy, nitro, carboxyl, cyano, sulfuryl and hydroxyl;
monocyclic aryl-alkyl having 5 to 7 ring members, which may have 1 to 2 heteroatoms selected from nitrogen, oxygen or sulfur, or substituted monocyclic aryl-alkyl having one or more substituents independently selected from amino, amidino, guanidino, hydrazino, C₁₋₄alkylamino, C₁₋₄dialkylamino, halogen, perfluoro C₁₋₄alkyl, C1-6alkyl, C₁₋₃alkoxy, nitro, carboxy, cyano, sulfuryl and hydroxyl;
bicyclic aryl-alkyl having 8 to 10 ring members, which may have 1 to 2 heteroatoms selected from nitrogen, oxygen or sulfur, or substituted bicyclic aryl-alkyl having one or more substituents independently selected from halogen, C₁₋₆alkyl, C₁₋₆alkoxy, cyano, hydroxyl; tricyclic aryl-alkyl having 5 to 14 ring members, which may have 1 to 2 heteroatoms selected from nitrogen, oxygen or sulfur, or substituted bicyclic aryl-alkyl having one or more substituents independently selected from halogen, C₁₋₆alkyl, C₁₋₆alkoxy, cyano, hydroxyl;
arylC₁₋₄alkyl or substituted arylC₁₋₄alkyl having one or more substituents independently selected from amino, amidino, guanidino, hydrazino, C₁₋₄alkylamino, C₁₋₄dialkylamino, C₃₋₆cycloalkyl, halogen, perfluoroC₁₋₄alkyl, C₁₋₆alkyl, C₁₋₃alkoxy, nitro, carboxyl, cyano, sulfuryl, hydroxyl, amide, C₁₋₆alkyloxyC₁₋₆acyl and morphorlinylC₁₋₆alkyl;
cycloalkylalkyl or substituted cycloalkylalkyl having one or more substituents independently selected from amino, amidino, guanidino, hydrazino, C₁₋₄alkylamino, C₁₋₄dialkylamino, halogen, perfluoro C₁₋₄alkyl, C₁₋₄alkyl, C₁₋₃alkoxy, nitro, carboxyl, cyano, sulfuryl and hydroxyl; and
cycloalkyl or substituted cycloalkyl having one or more substituents independently selected from amino, amidino, guanidino, hydrazino, C₁₋₄alkylamino, C₁₋₄dialkylamino, halogen, perfluoroC₁₋₄alkyl, C₁₋₄alkyl, C₁₋₃alkoxy, nitro, carboxyl, cyano, sulfuryl and hydroxyl.

In certain embodiments, R₁, R₂, R₃, R₄, and R₅ are the same or different and independently selected from the group consisting of:

Ci₋₁₂ alkyl or substituted Ĉ₁₂ alkyl having one or more substituents independently selected from amino, guanidino, C₁₋₄alkylguanidino, diC₁₋₄alkylguanidino, amidino, C1-4alkylamidino, diC1-4alkylamidino, C₁₋₅alkylamino, diC₁₋₅alkylamino, sulfide, carboxyl, hydroxyl; C₁₋₆alkoxy; cycloalkylC₁₋₃alkyl; cycloalkyl;
phenyl or substituted phenyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, hydroxyl;
phenylC₂₋₄alkyl or phenylC₂₋₄alkyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, sulfide, hydroxyl;
naphthyl or substituted naphthyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, hydroxyl;
naphthylC₁₋₄alkyl or naphthylC₁₋₄alkyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, hydroxyl;
benzyl or substituted benzyl having one or more substituents independently selected from amino, amidino, guanidino, hydrazino, C₁₋₄alkylamino, C₁₋₄diaalkylamino, halogen, perfluoro C₁₋₄ alkyl, trifluoroC₁₋₄alkyl; C₁₋₆alkyl, C₁₋₃alkoxy, nitro, carboxyl, cyano, sulfuryl and hydroxyl; bisphenylmethyl or substituted bisphenylmethyl having one or more substituents independently selected from amino, amidino, guanidino, hydrazino, C₁₋₄alkylamino, C₁₋₄ dialkylamino, halogen, perfluoro C₁₋₄alkyl, C₁₋₆alkyl, C₁₋₃alkoxy, nitro, carboxyl, cyano, sulfuryl and hydroxyl;
benzylphenyl amide, or substituted benzylphenyl amide having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;
pyridyl or substituted pyridyl having one or more substituents independently selected from amino, amidino, guanidino, hydrazino, C₁₋₄alkylamino, C₁₋₄dialkylamino,
halogen, perfluoro C₁₋₄alkyl, C₁₋₆alkyl, C₁₋₃alkoxy, nitro, carboxyl, cyano, sulfuryl and hydroxyl;
[50] pyridylC₁₋₄alkyl, or substituted pyridylC_{M}alkyl having one or more substituents independently selected from amino, amidino, guanidino, hydrazino, C₁₋₄alkylamino, C₁₋₄ dialkylamino, halogen, perfluoro C₁₋₄alkyl, C₁₋₄alkyl, C₁₋₃alkoxy, nitro, carboxyl, cyano, sulfuryl and hydroxyl;
pyrimidylC₁₋₄alkyl, or substituted pyrimidylC₁₋₄alkyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;
triazin-2-ylC₁₋₄alkyl, or substituted triazin-2-ylC₁₋₄alkyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;
imidazolylC₁₋₄alkyl or substituted imidazolylC₁₋₄alkyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;
benzothiazolinC₁₋₄alkyl or substituted benzothiazolinC₁₋₄alkyl having one or more substituents independently selected from amino, amidino, guanidino, hydrazino, C₁₋₄ alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;

phenoxazinC₁₋₄alkyl; benzyl p-tolyl ether; phenoxybenzyl; N-amidinopiperazinyl-N—C₁₋₄alkyl; quinolineC₁₋₄alkyl; N-amidinopiperazinyl; N-amidinopiperidinylC₁₋₄alkyl; 4-amino cyclohexylC₁₋₂alkyl; and 4-amino cyclohexyl.

In certain embodiments, E is —(ZR₄)— and G is —(XR₅)—, wherein Z is CH and X is nitrogen, and the compound has the following general formula (II):

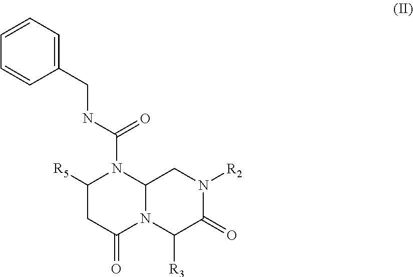

wherein R₂, R₃, and R₅ are as defined as in formula (I).

In certain embodiments, the compound has the following general formula (III):

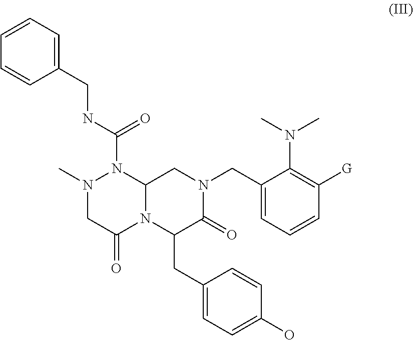

In certain embodiments, E is —(ZR₄)— and G is nothing, wherein Z is nitrogen, and the compound has the following general formula (IV):

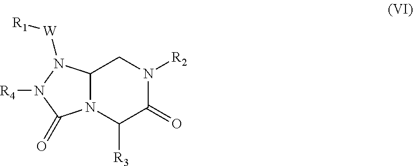

wherein R₁, R₂, R₃, R₄, and W are as defined in formula (I).

In certain embodiments, E is —(ZR₄)— and G is —(XR₅)—, wherein Z and X are independently CH, and the compound has a stricture of Formula (V):

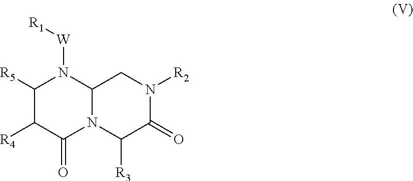

wherein R₁, R₂, R₃, R₄, R₅, and W are as defined in formula (I).

In certain embodiments, the compound has the following general formula (VI):

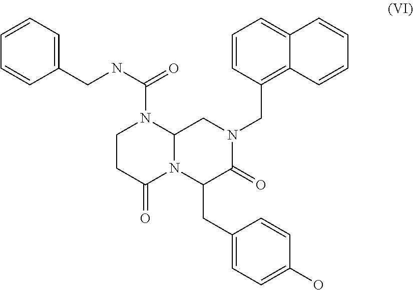

US 2004/0072831. All compound genera, species and conformations thereof of US 2004/0072831, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having formula (I):

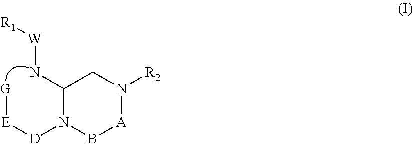

wherein: A is —(CHR₃)— or —(C═O)—, B is —(CHR₄)— or —(C═O)—, D is —(CHR₅)— or —(C═O)—, E is —(ZR₆)— or —(C═O)—, G is —(XR₇)ₙ—, —(CHR₇)—(NR₈)—, —(C═O)—(XR₉)—, or —(C═O)—, W is —Y(C═O)—, —(C═O)NH—, —(SO₂)— or is absent, Y is oxygen or sulfur, X and Z is independently nitrogen or CH, n=0 or 1; and R₁, R₂, R₃, R₄, R₅, R₆, R₇, R₈ and R₉ are the same or different and independently selected from an amino acid side chain moiety or derivative thereof, the remainder of the molecule, a linker and a solid support, and stereoisomers thereof, and as defined in US 2004/0072831.

In embodiments wherein A is —(CHR₃)—, B is —(C═O)—, D is —(CHR₅)—, E is —(C═O)—, and G is —(XR₇)ₙ—, the compounds of this invention have the following formula (II):

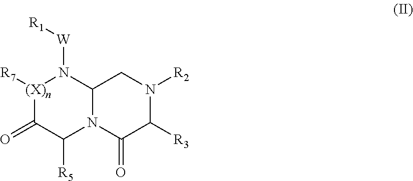

wherein W, X, Y and n are as defined above, and R₁, R₂, R₃, R₅ and R₇ are as defined in US 2004/0072831.

In embodiments wherein A is —(C═O)—, B is —(CHR₄)—, D is —(C═O)—, E is —(ZR₆)—, and G is —(C═O)—(XR₉)—, the compounds of this invention have the following formula (III):

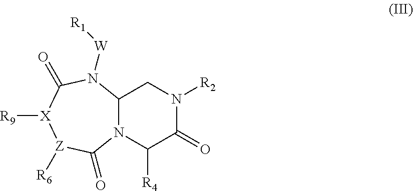

wherein W, X and Y are as defined above, Z is nitrogen or CH (with the proviso that when Z is CH, then X is nitrogen), and R₁, R₂, R₄, R₆ and R₉ are as defined in US 2004/0072831.

In embodiments wherein A is —(C═O)—, B is —(CHR₄)—, D is —(C═O)—, E is —(ZR₆)—, and G is (XR₇)ₙ—, the compounds of this invention have the following general formula (IV):

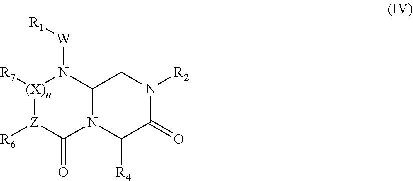

wherein W, Y and n are as defined above, Z is nitrogen or CH (when Z is nitrogen, then n is zero, and when Z is CH, then X is nitrogen and n is not zero), and R₁, R₂, R₄, R₆ and R₇, are as defined in US 2004/0072831.

In certain embodiments, wherein R₁, R₂, R₃, R₄, R₅, R₆, R₇, R₈ and R₉ are independently selected from the group consisting of aminoC₂₋₅alkyl, guanidinoC₂₋₅alkyl, C₁₋₄alkylguanidinoC₂₋₅alkyl, diC₁₋₄alkylguanidino-C₂₋₅alkyl, amidino C₂₋₅alkyl, C₁₋₄alkylamidino C₂₋₅alkyl, diC₁₋₄alkylamidinoC₂₋₅alkyl, C₁₋₃alkoxy, Phenyl, substituted phenyl(where the substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), benzyl, substituted benzyl (where the substituents on the benzyl are independently selected from one or more of amino, amidino, guanidino, hydrazino, amidazonyl, C₁₋₄alkylamino, Cli₄dialkylamino, halogen, perfluoro C₁₋₄alkyl, C₁₋₃alkoxy, nitro, carboxy, cyano, sulfuryl or hydroxyl), naphthyl, substituted naphthyl (where the substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), bis-phenyl methyl, substituted bis-phenyl methyl (where the substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋4alkyl, C₁₋₃alkoxy, nitro, carboxy, cyano, sulfuryl or hydroxyl), pyridyl, substituted pyridyl, (where the substituents are independently selected from one or more of amino amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridylC₁₋₄alkyl, substituted pyridylC₁₋₄alkyl (where the pyridine substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyrimidylC₁₋₄alkyl, substituted pyrimidylC₁₋₄alkyl (where the pyrimidine substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy or nitro, carboxy, cyano, sulfuryl or hydroxyl), triazin-2-yl-C₁₋₄alkyl, substituted triazin-2-yl-C₁₋₄alkyl (where the triazine substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), imidazoC₁₋₄alkyl, substituted imidazol C₁₋₄alkyl (where the imidazole substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), imidazolinylC₁₋₄alkyl, N-amidinopiperazinyl-N—C₀₋₄alkyl, hydroxyC₂₋₅alkyl, C₁₋₅alkylaminoC₂₋₅alkyl, hydroxyC₂₋₅alkyl, C₁₋₅alkylamino C₂₋₅alkyl, C₁₋₅dialkylamino C₂₋₅alkyl, N-amidinopiperidinylC₁₋₄alkyl and 4-aminocyclohexylC₀₋₂alkyl.

US 2007/0043052. All compound genera, species and conformations thereof of US 2007/0043052, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having formula (I):

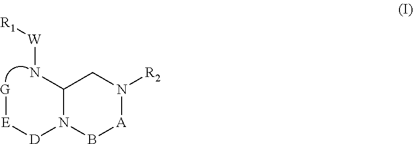

wherein: A is —(CHR₃)— or —(C═O)—, B is —(CHR₄)— or —(C═O)—, D is —(CHR₅)— or —(C═O)—, E is —(ZR₆)— or —(C═O)—, G is —(XR₇)ₙ—, —(CHR₇)—(NR₈)—, —(C═O)—(XR₉)—, or —(C═O)—, W is —Y(C═O)—, —(C═O)NH—, —(SO₂)— or is absent, Y is oxygen or sulfur, X and Z is independently nitrogen or CH, n=0 or 1; and R₁, R₂, R₃, R₄, R₅, R₆, R₇, R₈ and R₉ are the same or different and independently selected from an amino acid side chain moiety or derivative thereof, the remainder of the molecule, a linker and a solid support, and stereoisomers thereof, and as defined in US 2007/0043052.

In embodiments wherein A is —CHR₃)—, B is —(C═O)—, D is —(CHR₅)-E is —(C═O)—, and G is —XR₇)ₙ—, the compounds of this invention have the following formula (II):

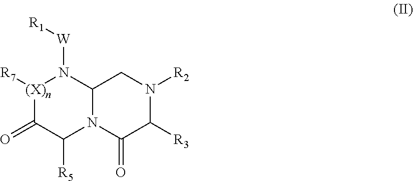

wherein W, X, Y and n are as defined above, and R₁, R₂, R₃, R₅ and R₇ are as defined in US 2007/0043052.

In embodiments wherein A is —(C═O)—, B is —(CHR₄)—, D is —(C═O)—, E is —(ZR₆)—, and G is —(C═O)—(XR₉)—, the compounds of this invention have the following formula (III):

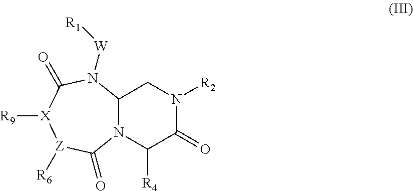

wherein W, X and Y are as defined above, Z is nitrogen or CH (with the proviso that when Z is CH, then X is nitrogen), and R₁, R₂, R₄, R₆ and R₉ are as defined in US 2007/0043052.

In embodiments wherein A is —(C═O)—, B is —(CHR₄)—, D is —(C═O)—, E is —(ZR₆)—, and G is —(XR₇)ₙ—, the compounds of this invention have the following general formula (IV):

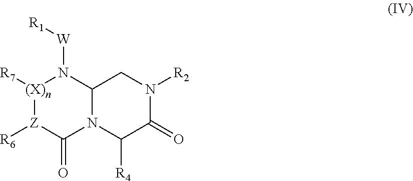

wherein W, Y and n are as defined above, Z is nitrogen or CH (when Z is nitrogen, then n is zero, and when Z is CH, then X is nitrogen and n is not zero), and R₁, R₂, R₄, R₆ and R₇, are as defined in US 2007/0043052.

In certain embodiments, the compounds of this invention have the following general formula (VI):

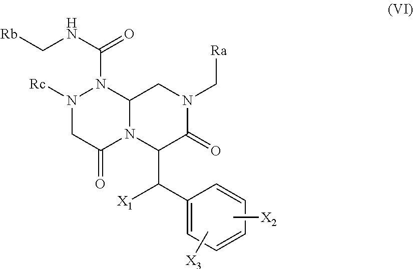

wherein Rₐ is a phenyl group; a substituted phenyl group having one or more substituents wherein the one or more substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl groups; a benzyl group; a substituted benzyl group with one or more substituents where the one or more substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl, and hydroxyl group; or a bicyclic aryl group having 8 to 11 ring members, which may have 1 to 3 heteroatoms selected from nitrogen, oxygen or sulfur; R_{b} is a monocyclic aryl group having 5 to 7 ring members, which may have 1 to 2 heteroatoms selected from nitrogen, oxygen or sulfur, and aryl ring in the compound may have one or more substituents selected from a group consisting of halide, hydroxy, cyano, lower alkyl, and lower alkoxy groups; R, is a saturated or unsaturated C₁₋₆alkyl, C₁₋₆alkoxy, perfluoro C₁₋₆alkyl group; and X₁, X₂, and X₃ may be the same or different and independently selected from hydrogen, hydroxyl, and halide.

In certain embodiments, prodrugs have the following general formula (VII):

—Y—R₁₀ (VI)

wherein (VI) is general formula (VI) as described above; Y is oxygen, sulfur, or nitrogen of a group selected from Rₐ, R_{b}, R_{c}, X₁, X₂ and X₃;
R₁₀ is phosphate, hemisuccinate, hemimalate, phosphoryloxymethyloxycarbonyl, dimethylaminoacetate, dimethylaminoalkylcarbamates, hydroxyalkyls, amino acid, glycosyl, substituted or unsubstituted piperidine oxycarbonyl, or a salt thereof; and wherein the prodrugs are capable of serving as a substrate for a phosphatase or a carboxylase and are thereby converted to compounds having general formula (VI).

In some embodiments, R₁₀ of the general formula (VII) is not an amino acid group or a phospho-amino acid group.

US 2005/0059628. All compound genera, species and conformations thereof of US 2005/0059628, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having formula (I):

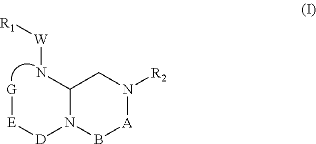

wherein: A is —(CHR₃)— or —(C═O)—, B is —(CHR₄)— or —(C═O)—, D is —(CHR₅)— or —(C═O)—, E is —(ZR₆)— or —(C═O)—, G is —(XR₇)ₙ—, —(CHR₇)—(NR₈)—, —(C═O)—(XR₉)—, or —(C═O)—, W is —Y(C═O)—, —(C═O)NH—, —(SO₂)— or is absent, Y is oxygen or sulfur, X and Z is independently nitrogen or CH, n=0 or 1; and R₁, R₂, R₃, R₄, R₅, R₆, R₇, R₈ and R₉ are the same or different and independently selected from an amino acid side chain moiety or derivative thereof, the remainder of the molecule, a linker and a solid support, and stereoisomers thereof, and as defined in US 2005/0059628.

In certain embodiments, R₁, R₂, R₃, R₄, R₅, R₆, R₇, R₈ and R₉ of formula (I) are independently selected from the group consisting of aminoC₂₋₅alkyl, guanidinoC₂₋₅alkyl, C₁₋₄alkylguanidinoC₂₋₅alkyl, diC₁₋₄alkylguanidino-C₂₋₅alkyl, amidinoC₂₋₅alkyl, C₁₋₄alkylamidinoC₂₋₅alkyl, diC₁₋₄alkylamidinoC₂₋₅alkyl, C₁₋₃alkoxy, phenyl, substituted phenyl(where the substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), benzyl, substituted benzyl (where the substituents on the benzyl are independently selected from one or more of amino, amidino, guanidino, hydrazino, amidazonyl, C₁₋₄alkylamino, C₁₋₄dialkylamino, halogen, perfluoro C₁₋₄alkyl, C₁₋₃alkoxy, nitro, carboxy, cyano, sulfuryl or hydroxyl), naphthyl, substituted naphthyl (where the substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), bis-phenyl methyl, substituted bis-phenyl methyl (where the substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridyl, substituted pyridyl, (where the substituents are independently selected from one or more of amino amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridylC₁₋₄alkyl, substituted pyridylC₁₋₄alkyl (where the pyridine substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyrimidylC₁₋₄alkyl, substituted pyrimidylC₁₋₄alkyl (where the pyrimidine substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy or nitro, carboxy, cyano, sulfuryl or hydroxyl), triazin-2-yl-C₁₋₄alkyl, substituted triazin-2-yl-C₁₋₄alkyl (where the triazine substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), imidazoC₁₋₄alkyl, substituted imidazol C₁₋₄alkyl (where the imidazole substituents are independently selected from one or more of amino, amidino, guanidino, hydrazino, amidazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), imidazolinylC₁₋₄alkyl, N-amidinopiperazinyl-N—C₀₋₄alkyl, hydroxyC₂₋₅alkyl, C₁₋₅alkylaminoC₂₋₅alkyl, hydroxyC₂₋₅alkyl, C₁₋₅alkylaminoC₂₋₅alkyl, C₁₋₅dialkylaminoC₂₋₅alkyl, N-amidinopiperidinylC₁₋₄alkyl and 4-amino cyclohexylC₀₋₂alkyl.

In certain embodiments, A is —(CHR₃)—, B is —(C═O)—, D is —(CHR₅)—, E is —(C═O)—, G is —(XR₇)ₙ—, and the compound has the following general formula (II):

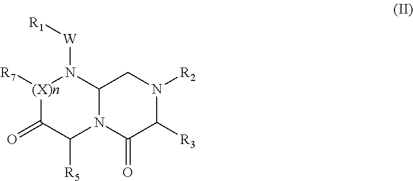

wherein R₁, R₂, R₃, R₅, R₇, W, X and n are as defined as in formula (I).

In certain embodiments, A is —(C═O)—, B is —(CHR₄)—, D is —(C═O)—, E is —(ZR₆)—, G is —(C═O)—(XR₉)—, and the compound has the following general formula (III):

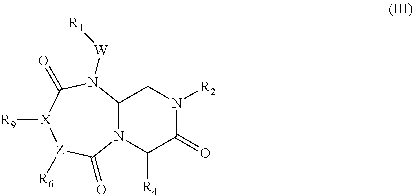

wherein R₁, R₂, R₄, R₆, R₉, W and X are as defined in formula (I), Z is nitrogen or CH (when Z is CH, then X is nitrogen).

In certain embodiments, A is —(C═O)—, B is —(CHR₄)—, D is —(C═O)—, E is —(ZR₆)—, G is —(XR₇)ₙ—, and the compound has the following general formula (IV):

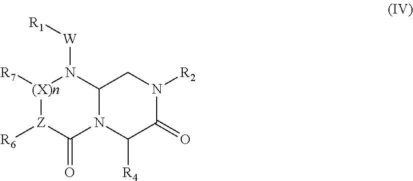

wherein R₁, R₂, R₄, R₆, R₇, W, X and n are as defined in formula (I), and Z is nitrogen or CH, with the proviso that when Z is nitrogen, then n is zero, and when Z is CH, then X is nitrogen and n is not zero.

In certain embodiments, the compound has the following general formula (VI):

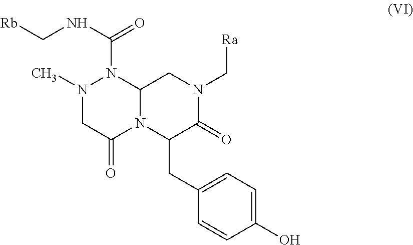

wherein, Rₐ is a bicyclic aryl group having 8 to 11 ring members, which may have 1 to 3 heteroatoms selected from nitrogen, oxygen or sulfur, and R_{b} is a monocyclic aryl group having 5 to 7 ring members, which may have 1 to 2 heteroatoms selected from nitrogen, oxygen or sulfur, and aryl ring in the compound may have one or more substituents selected from a group consisting of halide, hydroxy, cyano, lower alkyl, and lower alkoxy group. Optionally, Rₐ is naphthyl, quinolinyl or isoquinolinyl group, and R_{b} is phenyl, pyridyl or piperidyl, all of which may be substituted with one or more substituents selected from a group consisting of halide, hydroxy, cyano, lower alkyl, and lower alkoxy group. In certain embodiments, Rₐ is naphthyl, and R_{b} is phenyl, which may be substituted with one or more substituents selected from a group consisting of halide, hydroxy, cyano, lower alkyl, and lower alkoxy group.

In certain embodiments, the compound is selected from COMPOUNDS 1, 3, 4, and 5 as defined in US 2005/0059628.

WO 2009/051399. All compound genera, species and conformations thereof of WO 2009/051399, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having formula (I):

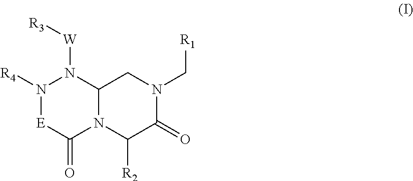

wherein E is —ZR3- or —(C═O)—, wherein Z is CH or N; W is —(C═O)—, —(C═O)NH—, —(C═O)O—, —(C═O)S—, —S(O)z- or a bond; and each of R₁, R₂, R₃, R4 and R₅ is the same or different and independently an amino acid side chain moiety or an amino acid side chain derivative. The reverse turn mimetic compound may be present as an isolated stereoisomer or a mixture of stereoisomers or as a pharmaceutically acceptable salt thereof. In certain embodiments, R₁ of compounds of Formula (I) is indazolyl or substituted indazolyl. Specific examples of R₁, R₂, R₃, R₄ and R₅ are as defined in WO 2009/051399.

In embodiments wherein E is CHR3, the compounds of this invention have the following Formula (II):

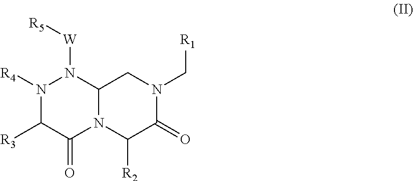

wherein W is as defined above, and R₁, R₂, R₃, R₄ and R₅ are as defined in WO 2009/051399.

In certain embodiments, the compounds of this invention have the following general Formula (III):

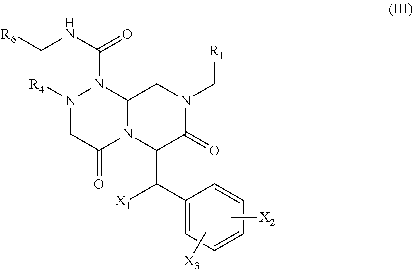

wherein R₁, R₄, R₆, X₁, X₂ and X₃ are as defined in WO 2009/051399.

In certain embodiments, the prodrugs of the present invention have the following general Formula (IV):

(III)-R₇ (IV)

wherein (III) is Formula (III) as described above; one of R₁, R₄, R₆, X₁, X₂ and X₃ is linked to R₇ via Y; Y is an oxygen, sulfur, or nitrogen in R₁, R₄ or R₆ or an oxygen in X₁, X₂, or X₃; and R7 is hydroxyalkyl, glycosyl, phosphoryloxymethyloxycarbonyl, substituted or unsubstituted piperidine carbonyloxy, or a salt thereof; or Y—R7 is an amino acid residue, a combination of amino acid residues, phosphate, hemimalate, hemisuccinate, dimethylaminoalkylcarbamate, dimethylaminoacetate, or a salt thereof; and when not linked to R₇: R₁, R₄, R₆, X₁, X₂ and X₃ are defined in WO 2009/051399.
US 2006/0084655. All compound genera, species and conformations thereof of US 2006/0084655, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having formula (I):

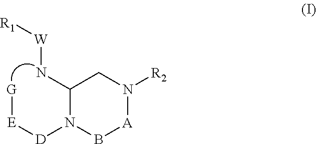

wherein: A is —(CHR₃)— or —(C═O)—, B is —(CHR₄)— or —(C═O)—, D is —(CHR₅)— or —(C═O)—, E is —(ZR₆)— or —(C═O)—, G is —(XR₇)ₙ—, —(CHR₇)—(NR₈)—, —(C═O)—(XR₉)—, or —(C═O)—, W is —Y(C═O)—, —(C═O)NH—, —(SO₂)— or is absent, Y is oxygen, sulfur, or —NH—, X and Z is independently nitrogen or CH, n=0 or 1; and R₁, R₂, R₃, R₄, R₅, R₆, R₇, R₈ and R₉ are the same or different and independently selected from an amino acid side chain moiety or derivative thereof, the remainder of the molecule, a linker and a solid support, and stereoisomers thereof, and as defined in US 2006/0084655.

In an embodiment wherein A is —(CHR₃)—, B is —(C═O)—, D is —(CHR₅)—, E is —(C═O)—, and G is —(XR₇)ₙ—, the compounds of this invention have the following formula (II):

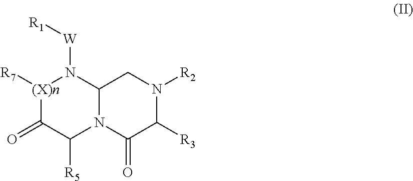

wherein W, X, Y and n are as defined above, and R₁, R₂, R₃, R₅ and R₇ are as defined in US 2006/0084655.

In an embodiment wherein A is —(C═O)—, B is —(CHR₄)—, D is —(C═O)—, E is —(ZR₆)—, and G is —(C═O)—(XR₉)—, the compounds of this invention have the following formula (III):

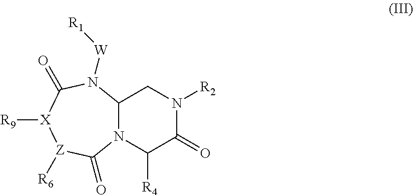

wherein W, X and Y are as defined above, Z is nitrogen or CH (with the proviso that when Z is CH, then X is nitrogen), and R₁, R₂, R₄, R₆ and R₉ are as defined in US 2006/0084655.

In an embodiment wherein A is —C═O)—, B is —(CHR₄)—, D is —(C═O)—, E is —(ZR₆)—, and G is (XR₇)ₙ—, the compounds of this invention have the following general formula (IV):

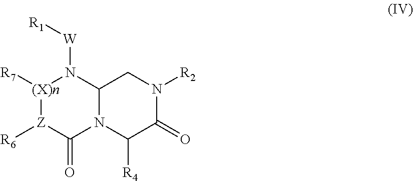

wherein W, Y and n are as defined above, Z is nitrogen or CH (when Z is nitrogen, then n is zero, and when Z is CH, then X is nitrogen and n is not zero), and R₁, R₂, R₄, R₆ and R₇, are as defined in US 2006/0084655.
US 2008/0009500. All compound genera, species and conformations thereof of US 2008/0009500, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having formula (I):

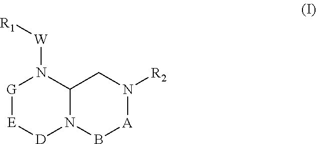

wherein A is —(C═O)—CHR3-, or —(C═O), B is N—R5- or —CHR6-, D is —(C═O)—(CHR7)- or —(C═O)—, E is —(ZR8)- or (C═O), G is —(XR9)n-, —(CHR10)-(NR6)-, —(C═O)—(XR12)-, -(or nothing)-, —(C═O)—, X—(C═O)—R13, X—(C═O)—NR13R14, X—(SO2)-R13, or X—(C═O)—OR13, W is —Y(C═O)—, —(C═O)NH—, (SO2)-, —CHR14, (C═O)—(NR15)-, substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidazole, or nothing, Y is oxygen or sulfur, X and Z is independently nitrogen or CH, n=0 or 1; and R1, R2, R3, R4, R5, R6, R7, R8, R9 R10, R11, R12, R13, R14, and R15 are the same or different and independently selected from an amino acid side chain moiety or derivative thereof, the remainder of the molecule, a linker and a solid support, and stereoisomers, salts, and prodrugs thereof, and a pharmaceutically acceptable carrier.

In certain embodiment, R1, R2, R3, R4, R5, R6, R7, R8, R9, R10, R11, R12, R13, R14, are R15 are independently selected from the group consisting of aminoC2-5alkyl, guanidinoC2-5alkyl, C1-4 alkylguanidino C2-5 alkyl, diC1-4 alkylguanidino-C2-5 alkyl, amidino C2-5 alkyl, C1-4alkylamidinoC2-5 alkyl, diC1- 4alkylamidinoC2-5 alkyl, C1-3alkoxy, phenyl, substituted phenyl (where the substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), benzyl, substituted benzyl (where the substituents on the benzyl are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), naphthyl, substituted naphthyl (where the substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), bis-phenyl methyl, substituted bis-phenyl methyl (where the substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), pyridyl, substituted pyridyl (where the substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), pyridylC1-4alkyl, substituted pyridylC1-4alkyl (where the pyridine substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), pyrimidylC1-4alkyl, substituted pyrimidylC1-4alkyl (where the pyrimidine substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy or nitro, carboxy, cyano, sulfuryl or hydroxyl), triazin-2-yl-C1-4alkyl, substituted triazin-2-yl-C1-4alkyl (where the triazine substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), imidazoC1-4alkyl, substituted imidazol C1-4alkyl (where the imidazole substituents are independently selected from one or more of amino, amidino, guanidino, hydrazine, amidrazonyl, C1-4alkylamino, C1-4dialkylamino, halogen, perfluoro C1-4alkyl, C1-4alkyl, C1-3alkoxy, nitro, carboxy, cyano, sulfuryl, hydroxyl, or methyl), imidazolinylC1-4alkyl, N-amidinopiperazinyl-N—C0-4alkyl, hydroxyC2-5alkyl, C1-5alkylaminoC2-5 alkyl, hydroxyC2-5 alkyl, C1-5 alkylamino C2-5 alkyl, C1-5 dialkylamino C2-5 alkyl, N-amidinopiperidinylC1-4alkyl and 4-aminocyclohexylC0-2alkyl.

In certain aspects, A is —(CHR3)-(C═O)—, B is —(NR4)-, D is —(C═O)—, E is —(ZR6)-, G is —(C═O)—(XR9)-, and the compound has the following general formula (III):

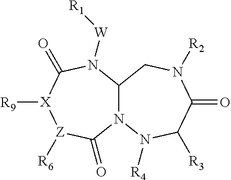

wherein Z is nitrogen or CH, and when Z is CH, X is nitrogen.

In certain aspects, A is —O—CHR3, B is —NR4, D is —(C═O)—, E is —(ZR6)-, Gi is (XR7)n-, the compound has the following formula (IV):

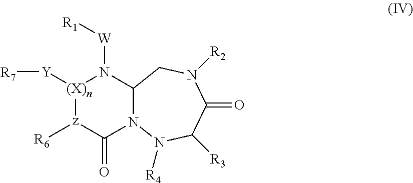

wherein R1, R2, R4, R6, R7, R8 W, X and n are as defined above, Y is —C═O, —(C═O)—O—, —(C═O)—NR8, —SO2-, or nothing, and Z is nitrogen or CH (when Z is nitrogen, then n is zero, and when Z is CH, then X is nitrogen and n is not zero).

In certain embodiment, when A is —(C═O)—, B is —(CHR6)-, D is —(C═O)—, E is —(ZR8)-, and G is —(NH)— or —(CH2)-, and W is a substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidazole, the compound has the following formula (V):

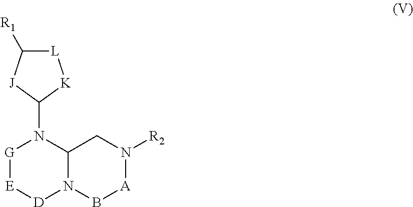

wherein K is nitrogen, oxygen, or sulfur, L is nitrogen, oxygen, —(CH)—, or —(CH2)-, J is nitrogen, oxygen, or sulfur, Z is nitrogen or CH, and R1, R2, R6, R8, and R13 are selected from an amino acid side chain moiety.

Particular embodiments provide a compound having the general formula (VI):

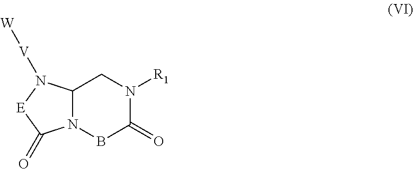

wherein B is —(CHR2)-, —(NR2)-, E is —(CHR3)-, V is —(XR4)- or nothing, W is —(C═O)—(XR5R6), —(SO2)-, substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidazole, X is independently nitrogen, oxygen, or CH, and R1, R2, R3, R4, R5 and R6 are selected from an amino acid side chain moiety or derivative thereof, the remainder of the molecule, a linker and solid support, and stereoisomers, salts and prodrugs thereof. In certain aspects, R1, R2, R3, R4, R5, R6, R7, R8, R9, R10, R11, R12, R13, R14, are R15 are independently selected from the group consisting of aminoC2-5alkyl, guanidinoC2-5alkyl, C1-4 alkylguanidino C2-5 alkyl, diC1-4 alkylguanidino-C2-5 alkyl, amidino C2-5 alkyl, C1-4alkylamidinoC2-5 alkyl, diC1-4alkylamidinoC2-5 alkyl, C1-3alkoxy, phenyl, substituted phenyl (where the substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), benzyl, substituted benzyl (where the substituents on the benzyl are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), naphthyl, substituted naphthyl (where the substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), naphthyl, substituted naphthyl (where the substituents are independently selected from one or more of amino, amidino, guanidine, hydrazino, amidrazonyl, C1-4 alkylamino, C1-4 dialkylamino, halogen, perfluoro C1-4 alkyl, C1-4 alkyl, C1-3 alkoxy, nitro, carboxy, cyano, sulfuryl or hydroxyl), bis-phenyl methyl, substituted bis-phenyl methyl (where the substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), pyridyl, substituted pyridyl (where the substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), pyridylC1-4alkyl, substituted pyridylC1-4alkyl (where the pyridine substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), pyrimidylC1-4alkyl, substituted pyrimidylC1-4alkyl (where the pyrimidine substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy or nitro, carboxy, cyano, sulfuryl or hydroxyl), triazin-2-yl-C1-4alkyl, substituted triazin-2-yl-C1-4alkyl (where the triazine substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3 alkoxy, nitro, carboxy, cyano, sulfuryl or hydroxyl), imidazoC1-4alkyl, substituted imidazol C1-4alkyl (where the imidazole substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl, hydroxyl, or methyl), imidazolinylC1-4alkyl, N-amidinopiperazinyl-N—C0-4 alkyl, hydroxyC2-5 alkyl, C1-5 alkylamino C2-5 alkyl, hydroxyC2-5 alkyl, C1-5 alkylamino C2-5 alkyl, C1-5 dialkylamino C2-5 alkyl, N-amidinopiperidinylC1-4alkyl and 4-amino cyclohexylC0-2alkyl.

In certain aspects, wherein B is —(CH)—(CH3), E is —(CH)—(CH3), V is —(XR4)- or nothing, and W is substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidazole, and X is independently nitrogen or CH, the compounds have the following general formula (VII):

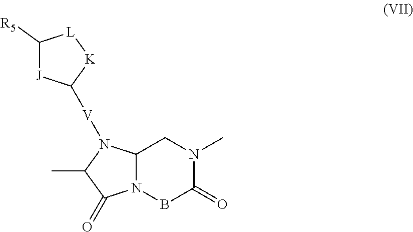

wherein K is nitrogen, oxygen, or sulfur, L is nitrogen, oxygen, —(CH)—, or —(CH2)-, J is nitrogen, oxygen, or sulfur, and R5 is independently selected from the group consisting of aminoC2-5 alkyl, guanidinoC2-5 alkyl, C1-4alkylguanidinoC2-5 alkyl, diC1-4alkylguanidino-C2-5 alkyl, amidino C2-5 alkyl, C1-4 alkylamidino C2-5 alkyl, diC1-4 alkylamidino C2-5 alkyl, C1-3alkoxy, Phenyl, substituted phenyl (where the substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), benzyl, substituted benzyl (where the substituents on the benzyl are independently selected from one or more of amino, amidino, guanidino, hydrazino, amidrazonyl, C1-4alkylamino, C1-4dialkylamino, halogen, perfluoro C1-4alkyl, C1-4alkyl, C1-3 alkoxy, nitro, carboxy, cyano, sulfuryl or hydroxyl), naphthyl, substituted naphthyl (where the substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3 alkoxy, nitro, carboxy, cyano, sulfuryl or hydroxyl), bis-phenyl methyl, substituted bis-phenyl methyl (where the substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), pyridyl, substituted pyridyl, (where the substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), pyridylC1-4alkyl, substituted pyridylC1-4alkyl (where the pyridine substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl or hydroxyl), pyrimidylC1-4alkyl, substituted pyrimidylC1-4alkyl (where the pyrimidine substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy or nitro, carboxy, cyano, sulfuryl or hydroxyl), triazin-2-yl-C1-4alkyl, substituted triazin-2-yl-C1-4alkyl (where the triazine substituents are independently selected from one or more of amino, amidino, guanidino, hydrazino, amidrazonyl, C1-4 alkylamino, C1-4 dialkylamino, halogen, perfluoro C1-4 alkyl, C1-4 alkyl, C1-3 alkoxy, nitro, carboxy, cyano, sulfuryl or hydroxyl), imidazoC1-4alkyl, substituted imidazol C1-4alkyl (where the imidazole substituents are independently selected from one or more of amino, amidino, guanidino, hydrazino, amidrazonyl, C1-4alkylamino, C1-4dialkylamino, halogen, perfluoro C1-4alkyl, C1-4alkyl, C1-3alkoxy, nitro, carboxy, cyano, sulfuryl, hydroxyl, or methyl), imidazolinylC1-4 alkyl, N-amidinopiperazinyl-N—C0-4 alkyl, hydroxyC2-5 alkyl, C1-5 alkylamino C2-5 alkyl, hydroxyC2-5 alkyl, C1-5 alkylamino C2-5 alkyl, C1-5 dialkylamino C2-5 alkyl, N-amidinopiperidinylC1-4alkyl and 4-amino cyclohexylC0-2alkyl.

Additional compounds comprise one selected from the group consisting of Compounds 1-2217 in 2008/0009500.

US 2010/0222303. All compound genera, species and conformations thereof of US 2010/0222303, including the exemplary compounds of Tables 3-5 thereof, the claimed compounds, and including the disclosed respective syntheses, are incorporated herein by reference in their entirety as exemplary compounds for use in applicant's presently claimed methods.

Specific exemplary embodiments include a compound having the structure:

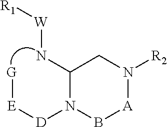

or a pharmaceutically acceptable salt or stereoisomer thereof,
wherein A is —(CHR₃)— or —(C═O)—, B is —(CHR₄)— or —(C═O)—, D is —(CHR₅)— or —(C═O)—, E is —(ZR₆)— or —(C═O)—, G is —(XR₇)ₙ—, —(CHR₇)—(NR₈)—, —(C═O)—(XR₉)—, or —(C═O)—, W is —Y(C═O)—, —(C═O)NH—, —(SO₂)— or is absent, Y is oxygen, sulfur, or —NH—, X and Z is independently nitrogen or CH, n=0 or 1; and R₁, R₂, R₃, R₄, R₅, R₆, R₇, R₈ and R₉ are the same or different and independently selected from an amino acid side chain moiety or derivative thereof, the remainder of the molecule, a linker and a solid support, and stereoisomers thereof, and as defined in US 2010/0222303.

In embodiments wherein A is —(CHR₃)— or —(C═O)—; B is —(CHR₄)— or —(C═O)—; D is —(CHR₅)— or —(C═O)—; E is —ZR₆— or —(C═O)—, wherein Z is CH or N; G is —XR₇— or —(C═O)—, wherein X is CH or N; W is —(C═O)NH—, —(C═O)O—, —(C═O)S—, —S(O)₂— or nothing; and each of R₁, R₂, R₃, R₄, R₅, R₆ and R₇ is the same or different and independently an amino acid side chain moiety or an amino acid side chain derivative, the compounds of this invention have the following formula (IA):

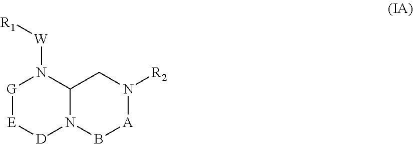

wherein specific examples of R₁, R₂, R₃, R₄, R₅, R₆ and R₇ are as defined in US 2010/0222303.

In embodiments wherein A is —(CHR₃)—, B is —(C═O)—, D is —(CHR₅)—, E is —(C═O)—, and G is —(XR₇)ₙ—, the compounds of this invention have the following formula (II):

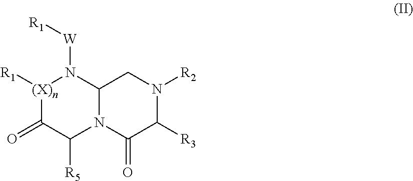

wherein W, X, Y and n are as defined above, and R₁, R₂, R₃, R₅ and R₇ are as defined in US 2010/0222303.

In embodiments wherein A is —(C═O)—, B is —(CHR₄)—, D is —(C═O)—, E is —(ZR₆)—, and G is —(C═O)—(XR₉)—, the compounds of this invention have the following formula (III):

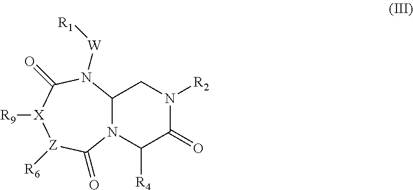

wherein W, X and Y are as defined above, Z is nitrogen or CH (with the proviso that when Z is CH, then X is nitrogen), and R₁, R₂, R₄, R₆ and R₉ are as defined in US 2010/0222303.

In embodiments wherein A is —(C═O)—, B is —(CHR₄)—, D is —(C═O)—, E is —(ZR₆)—, and G is —(XR₇)ₙ—, the compounds of this invention have the following general formula (IV):

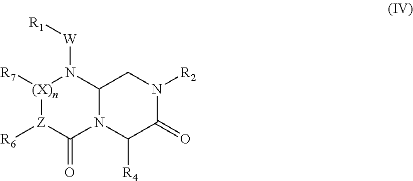

wherein W, Y and n are as defined above, Z is nitrogen or CH (when Z is nitrogen, then n is zero, and when Z is CH, then X is nitrogen and n is not zero), and R₁, R₂, R₄, R₆ and R₇, are as defined in US 2010/0222303.

In embodiments wherein A is —(C═O)—, B is —(CHR₄)—, D is —(C═O)—, E is —CHR₆—, and G is —XR₇—, wherein X is CH or N, and the compound has a structure of Formula (IVA):

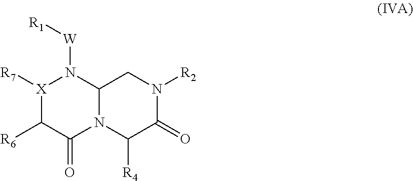

wherein R₁, R₂, R₄, R₆ and R₇ are as defined in US 2010/0222303.

In an embodiment of compounds of formula (IVA) wherein X is N, the compound has a structure of Formula (IVA₁):

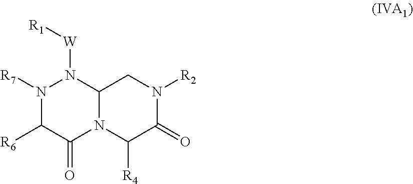

wherein R₁, R₂, R₄, R₆, R₇ are as defined in US 2010/0222303.

In certain embodiments, the compounds of this invention have the following general formula (VI):

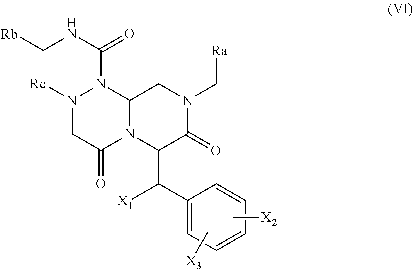

wherein Rₐ, R_{b}, and R, are as defined in US 2010/0222303, and X₁, X₂, and X₃ may be the same or different and independently selected from hydrogen, hydroxyl, and halide.
U.S. Pat. No. 6,413,963. All compound genera, species and conformations thereof of U.S. Pat. No. 6,413,963, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having formula (I):

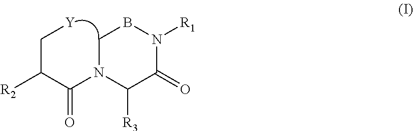

wherein Y is selected from —CH(R₅)-A-N(R₁)—, -A-N(R₁)—CH(R′)—, -A-N(R₁)—C(═O)—, -A-C(═O)—N(R₁)—, -A-CH(R₁)—O—, and -A-CH(R₁)N(R′)—; A is —(CHR′)ₙ—; B is —(CHR″)ₘ—; n=0, 1 or 2; m=1, 2 or 3; and any two adjacent CH groups or adjacent NH and CH groups on the bicyclic ring may optionally form a double bond; and wherein R′, R″, R₁, R₂, R₃, R₄ and R₅ are as defined in U.S. Pat. No. 6,413,963.

In embodiments wherein Y is —CH(R₅)-A-N(R₁)—, the compounds of this invention have the following structure (I′):

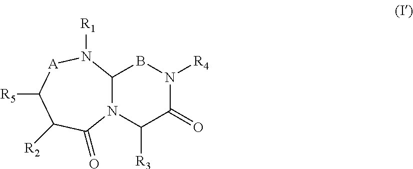

wherein A and B are as defined above, and R₁, R₂, R₃, R₄ and R₅ are as defined in U.S. Pat. No. 6,413,963.

In embodiments wherein Y is -A-N(R₁)—CH(R′)—, the compounds of this invention have the following structure (I″):

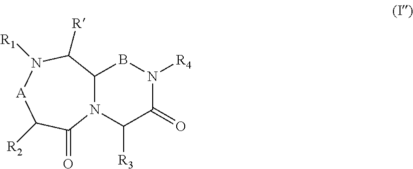

wherein A and B are as defined above, and R′, R₁, R₂, R₃ and R₄ are as defined in U.S. Pat. No. 6,413,963.

In embodiments wherein Y is -A-N(R₁)—C(═O)—, the compounds of this invention have the following structure (I′″):

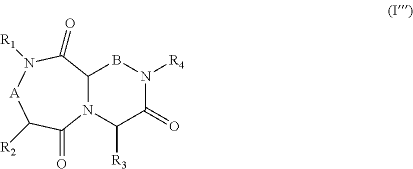

wherein A and B are as defined above, and R₁, R₂, R₃ and R₄ are as defined in U.S. Pat. No. 6,413,963.

In embodiments wherein Y is -A-C(═O)—N(R₁)—, the compounds of this invention have the following structure (I″″):

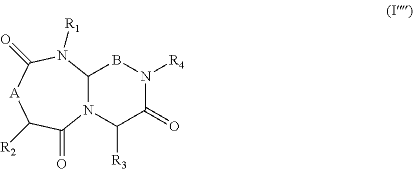

wherein A and B are as defined above, and R₁, R₂, R₃ and R₄ are as defined in U.S. Pat. No. 6,413,963.

In embodiments wherein Y is -A-CH(R₁)—O—, the compounds of this invention have the following structure (I′″″):

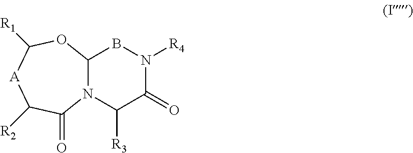

wherein A and B are as defined above, and R₁, R₂, R₃ and R₄ are as defined in U.S. Pat. No. 6,413,963.

In embodiments wherein Y is -A-CH(R₁)N(R′)—, the compounds of this invention have the following structure (I″″″):

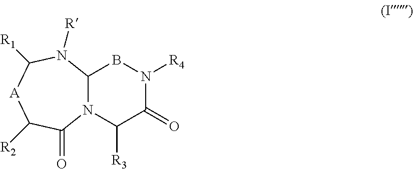

wherein A and B are as defined above, and R′, R₁, R₂, R₃ and R₄ are as defined in U.S. Pat. No. 6,413,963.
U.S. Pat. No. 7,531,320. All compound genera, species and conformations thereof of U.S. Pat. No. 7,531,320, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having formula (I):

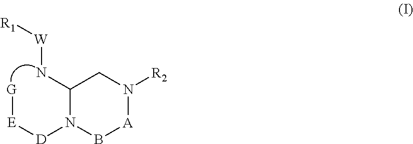

wherein A is —(CHR₃)— or —(C═O)—, B is —(CHR₄)— or —(C═O)—, D is —(CHR₅)— or —(C═O)—, E is —(ZR₆)— or —(C═O)—, G is —(XR₇)ₙ—, —(CHR₇)—(NR₈)—, —(C═O)—(XR₉)—, or —(C═O)—, W is —Y(C═O)—, —(C═O)NH—, —(SO₂)— or nothing, Y is oxygen or sulfur, X and Z is independently nitrogen or CH, n=0 or 1; and R₁, R₂, R₃, R₄, R₅, R₆, R₇, R₈ and R₉ are the same or different and are each independently selected from an amino acid side chain moiety, a derivative of an amino acid side chain moiety, or the remainder of the molecule, and stereoisomers thereof.

In certain embodiments, R₁, R₂, R₃, R₄, R₅, R₆, R₇, R₈ and R₉ of formula (I) are independently selected from the group consisting of aminoC₂₋₅ alkyl, guanidinoC₂₋₅ alkyl, C₁₋₄ alkylguanidinoC₂₋₅ alkyl, diC₁₋₄ alkylguanidino-C₂₋₅ alkyl, amidinoC₂₋₅ alkyl, C₁₋₄ alkylamidinoC₂₋₅ alkyl, diC₁₋₄ alkylamidinoC₂₋₅ alkyl, C₁₋₃ alkoxy, phenyl, substituted phenyl(where the substituents are independently selected from one or more of amino, amidino, guanidino, hydrazino, amidazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), benzyl, substituted benzyl (where the substituents on the benzyl are independently selected from one or more of amino, amidino, guanidino, hydrazino, amidazonyl, C₁₋₄ alkylamino, C₁₋₄ dialkylamino, halogen, perfluoro C₁₋₄ alkyl, C₁₋₃ alkoxy, nitro, carboxy, cyano, sulfuryl or hydroxyl), naphthyl, substituted naphthyl (where the substituents are independently selected from one or more of amino, amidino, guanidino, hydrazino, amidazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), bis-phenyl methyl, substituted bis-phenyl methyl (where the substituents are independently selected from one or more of amino, amidino, guanidino, hydrazino, amidazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), pyridyl, substituted pyridyl, (where the substituents are independently selected from one or more of amino amidino, guanidino, hydrazino, amidazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), pyridylC₁₋₄ alkyl, substituted pyridylC₁₋₄ alkyl (where the pyridine substituents are independently selected from one or more of amino, amidino, guanidino, hydrazino, amidazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), pyrimidylC₁₋₄ alkyl, substituted pyrimidylC₁₋₄ alkyl (where the pyrimidine substituents are independently selected from one or more of amino, amidino, guanidino, hydrazino, amidazonyl, C₁₋₄ alkylamino, C₁₋₄ dialkylamino, halogen, perfluoro C₁₋₄ alkyl, C₁₋₄ alkyl, C₁₋₃ alkoxy or nitro, carboxy, cyano, sulfuryl or hydroxyl), triazin-2-yl-C₁₋₄ alkyl, substituted triazin-2-yl-C₁₋₄ alkyl (where the triazine substituents are independently selected from one or more of amino, amidino, guanidino, hydrazino, amidazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), imidazoC₁₋₄ alkyl, substituted imidazol C₁₋₄ alkyl (where the imidazole substituents are independently selected from one or more of amino, amidino, guanidino, hydrazino, amidazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), imidazolinylC₁₋₄ alkyl, N-amidinopiperazinyl-N—C₀₋₄ alkyl, hydroxyC₂₋₅ alkyl, C₁₋₅ alkylaminoC₂₋₅ alkyl, hydroxyC₂₋₅ alkyl, C₁₋₅ alkylaminoC₂₋₅ alkyl, C₁₋₅ dialkylaminoC₂₋₅ alkyl, N-amidinopiperidinylC₁₋₄ alkyl and 4-aminocyclohexylC₀₋₂ alkyl.

In certain embodiments, A is —(CHR₃)—, B is —(C═O)—, D is —(CHR₅)—, E is —(C═O)—, G is —(XR₇)ₙ—, and the compound has the following general formula (II):

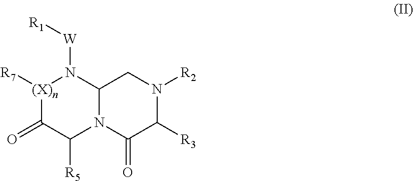

wherein R₁, R₂, R₃, R₅, R₇, W, X and n are as defined as in formula (I).

In certain embodiments, A is —(C═O)—, B is —(CHR₄)—, D is —(C═O)—, E is —(ZR₆)—, G is —(C═O)—(XR₉)—, and the compound has the following general formula (III):

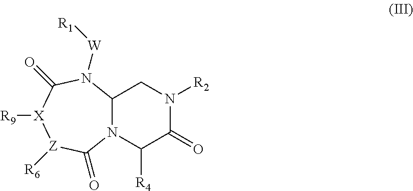

wherein R₁, R₂, R₄, R₆, R₉, W and X are as defined in formula (I), Z is nitrogen or CH (when Z is CH, then X is nitrogen).

In certain embodiments, A is —(C═O)—, B is —(CHR₄)—, D is —(C═O)—, E is —(ZR₆)—, G is —(XR₇)ₙ—, and the compound has the following general formula (IV):

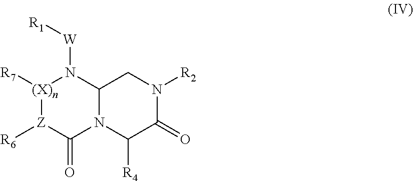

wherein R₁, R₂, R₄, R₆, R₇, W, X and n are as defined in formula (I), and Z is nitrogen or CH, with the proviso that when Z is nitrogen, then n is zero, and when Z is CH, then X is nitrogen and n is not zero.

In certain embodiments, the compound has the following general formula (VI):

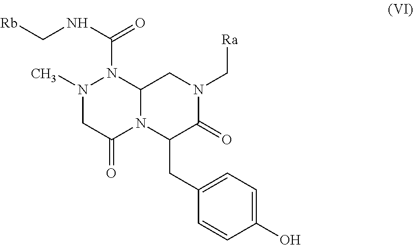

wherein, Rₐ is a bicyclic aryl group having 8 to 11 ring members, which may have 1 to 3 heteroatoms selected from nitrogen, oxygen or sulfur, and R_{b} is a monocyclic aryl group having 5 to 7 ring members, which may have 1 to 2 heteroatoms selected from nitrogen, oxygen or sulfur, and aryl ring in the compound may have one or more substituents selected from a group consisting of halide, hydroxy, cyano, lower alkyl, and lower alkoxy group. Optionally, Rₐ is naphthyl, quinolinyl or isoquinolinyl group, and R_{b} is phenyl, pyridyl or piperidyl, all of which may be substituted with one or more substituents selected from a group consisting of halide, hydroxy, cyano, lower alkyl, and lower alkoxy group. In certain embodiments, Rₐ is naphthyl, and R_{b} is phenyl, which may be substituted with one or more substituents selected from a group consisting of halide, hydroxy, cyano, lower alkyl, and lower alkoxy group.

In certain embodiments, the compound is selected from COMPOUNDS 1, 3, 4, and 5 as defined in U.S. Pat. No. 6,413,963.

U.S. Pat. No. 7,563,825. All compound genera, species and conformations thereof of U.S. Pat. No. 7,563,825, including the exemplary compounds of Tables 1-5 thereof, the claimed compounds, and including the disclosed respective syntheses, are incorporated herein by reference in their entirety as exemplary compounds for use in applicant's presently claimed methods.

Specific exemplary embodiments include a compound having formula (I):

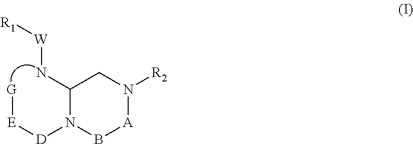

wherein A is —(C═O)—(CHR₃)—, B is —N—R₄—, D is —(CHR₅)— or —(C═O)—, E is —(ZR₆)— or —(C═O)—, G is —(XR₇)ₙ—, —(CHR₇)—(NR₈)—, —(C═O)—(XR₉)—, or —(C═O)—, W is —Y(C═O)—, —(C═O)NH—, —(SO₂)— or nothing, Y is oxygen or sulfur, X and Z is independently nitrogen or CH, n=0 or 1; and R₁, R₂, R₃, R₄, R₅, R₆, R₇, R₈ and R₉, are the same or different and independently selected from an amino acid side chain moiety or derivative thereof, the remainder of the molecule, a linker and a solid support, and stereoisomers thereof. More specifically, R₁, R₂, R₃, R₄, R₅, R₆, R₇, R₈ and R₉, are independently selected from the group consisting of aminoC₂₋₅ alkyl, guanidineC₂₋₅ alkyl, C₁₋₄ alkylguanidinoC₂₋₅ alkyl, diC₁₋₄ alkylguanidino-C₂₋₅ alkyl, amidinoC₂₋₅ alkyl, C₁₋₄ alkylamidino C₂₋₅ alkyl, diC₁₋₄ alkylamidinoC₂₋₅ alkyl, C₁₋₃ alkoxy, Phenyl, substituted phenyl (where the substituents are independently selected from one or more of amino, amidino, guanidino, hydrazino, amidrazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), benzyl, substituted benzyl (where the substituents on the benzyl are independently selected from one or more of amino, amidino, guanidino, hydrazino, amidrazonyl, C₁₋₄ alkylamino, C₁₋₄ dialkylamino, halogen, perfluoro C₁₋₄ alkyl, C₁₋₃ alkyl, nitro, carboxy, cyano, sulfuryl or hydroxyl), naphthyl, substituted naphthyl (where the substituents are independently selected from one or more of amino, amidino, guanidino, hydrazino, amidrazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), bisphenyl methyl, substituted bis-phenyl methyl (where the substituents are independently selected from one or more of amino, amidino, guanidino, hydrazino, amidrazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), pyridyl, substituted pyridyl, (where the substituents are independently selected from one or more of amino, amidino, guanidino, hydrazino, amidrazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), pyridylC₁₋₄ alkyl, substituted pyridylC₁₋₄ alkyl (where the pyridine substituents are independently selected from one or more of amino, amidino, guanidino, hydrazino, amidrazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), pyrimidylC₁₋₄ alkyl, substituted pyrimidylC₁₋₄ alkyl (where the pyrimidine substituents are independently selected from one or more of amino, amidino, guanidino, hydrazino, amidrazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), triazin-2-yl-C₁₋₄ alkyl, substituted triazin-2-yl-C₁₋₄ alkyl (where the triazine substituents are independently selected from one or more of amino, amidino, guanidino, hydrazino, amidrazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), imidazoC₁₋₄ alkyl, substituted imidazol C₁₋₄ alkyl (where the imidazole substituents are independently selected from one or more of amino, amidino, guanidino, hydrazino, amidrazonyl, C₁₋₄ alkylamino, C₁₋₄ dialkylamino, halogen, perfluoro C₁₋₄ alkyl, C₁₋₄ alkyl, C₁₋₃ alkoxy, nitro, carboxy, cyano, sulfuryl or hydroxyl), imidazolinylCalkyl, N-amidinopiperazinyl-N—C₀₋₄ alkyl, hydroxyC₂₋₅ alkyl, C₁₋₅ alkylaminoC₂₋₅ alkyl, hydroxyC₂₋₅ alkyl, C₁₋₅ alkylaminoC₂₋₅ alkyl, C₁₋₅ dialkylaminoC₂₋₅ alkyl, N-amidinopiperidinylC₁₋₄ alkyl and 4-aminocyclohexylC₀₋₂ alkyl.

In one embodiment, R₁, R₂, R₆ of E, and R₇, R₈and R₉ of G are the same or different and represent the remainder of the compound, and R₃ or A, R₄ of B or R₅ of D is selected from an amino acid side chain moiety or derivative thereof. As used herein, the term “remainder of the compound” means any moiety, agent, compound, support, molecule, linker, amino acid, peptide or protein covalently attached to the α-helix mimetic structure at R₁, R₂, R₅, R₆, R₇, R₈ and/or R₉ positions. This term also includes amino acid side chain moieties and derivatives thereof, as defined in U.S. Pat. No. 7,563,825.

In embodiments wherein A is —(C═O)—CHR₃—, B is —N—R₄, D is —(C═O)—, E is —(ZR₆)—, G is —(C═O)—(XR₉)—, the α-helix mimetic compounds for use in this invention have the following general formula (III):

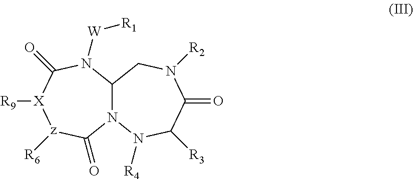

wherein R₁, R₂, R₄, R₆, R₉, W and X are as defined above, Z is nitrogen or CH (when Z is CH, then X is nitrogen). In a preferred embodiment, R₁, R₂, R₆, and R₉ represent the remainder of the compound, and R₄ is selected from an amino acid side chain moiety. In a more specific embodiment wherein A is —O—CHR₃—, B is —NR₄—, D is —(C═O)—, E is —(ZR₆)—, Gi is (XR₇)ₙ—, the α-helix mimetic compounds for use in this invention have the following formula (IV):

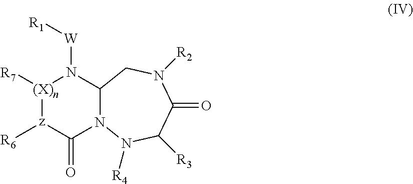

wherein R₁, R₂, R₄, R₆, R₇, W, X and n are as defined above, and Z is nitrogen or CH (when Z is nitrogen, then n is zero, and when Z is CH, then X is nitrogen and n is not zero). In a preferred embodiment, R₁, R₂, R₆, and R₇ represent the remainder of the compound, and R₄ is selected from an amino acid side chain moiety. In this case, R₆ or R₇ may be selected from an amino acid side chain moiety when Z and X are CH, respectively.

### Further Exemplary Compounds

In particular exemplary aspects, the CBP/β-catenin antagonists (e.g., of TABLE 1) comprises ICG-001, and salts (e.g., physiologically acceptable salts) and derivatives thereof having activity in the methods disclosed herein.

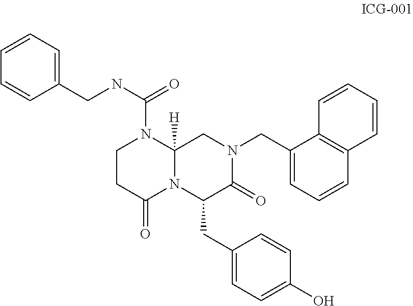 [SMILES: O=C1[C@H](Cc2ccc(O)cc2)N2C(=O)CCN(C(=O)NCc3ccccc3)[C@H]2CN1Cc1cccc2ccccc12]

In particular aspects, alkyl variants and/or derivatives of the useful CBP/β-catenin antagonists of TABLE 1 are used, such as:

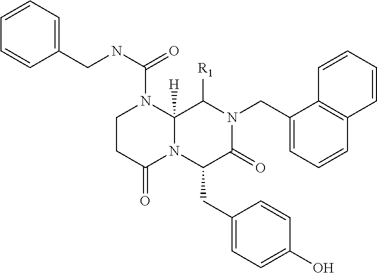

wherein R₁ is selected from C1-C6 alkyl, wherein the adjoining moiety (here an exemplary bicyclic moiety) on the ring can be any of the substitutions at this position exemplified by the compounds of TABLE 1. In preferred aspects R₁ is CH₃. In particular aspects, R₁ has the following conformation:

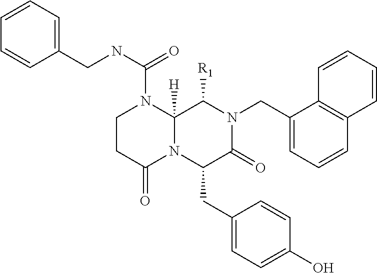

See also PCT/US2011/061062 (published as WO 2012/068299 A2), incorporated by reference herein in its entirety.

### Pharmaceutical Compositions

Additional aspects provide a pharmaceutical composition comprising a compound according to any one of the above compounds, or a pharmaceutically acceptable salt thereof, and optionally a pharmaceutically acceptable carrier. In particular aspects, the pharmaceutical composition comprises an effective amount of the compound. In certain aspects, the compound is a CBP/β-catenin antagonist. In particular aspects, the effective amount of the CBP/β-catenin antagonist is sufficient to promote differentiation of an amplified somatic stem cell pool as described and claimed herein.

### Dosages

Administration of the CBP/catenin (e.g., CBP/β-catenin) antagonist may comprise topical administration (e.g., 100 μM to 2 mM). Alternatively, the compounds of the present invention can be administered intravenously (e.g., continuous drip infusion or rapid intravenous administration) to mammals inclusive of human. The dose, as will be recognized in the art, is selected appropriately depending on various factors such as the body weight and/or age of patients, and/or the degree of the symptom and an administration route. For example, the dose for oral or intravenous administration is generally in the range of 1 to 10000 mg/day/m² human body surface area, preferably in the range of 1 to 5000 mg/day/m² human body surface area, and more preferably 10 to 5000 mg/day/m² human. The dose range may be between 50 mg/m2/day-1000 mg/m2/day (for i.v.) and 5-100 mg/kg day (for oral).

### Prodrugs

The present invention is also related to prodrugs using the libraries containing one or more compounds of formula (I). A prodrug is typically designed to release the active drug in the body during or after absorption by enzymatic and/or chemical hydrolysis. The prodrug approach is an effective means of improving the topical, oral, etc., bioavailability or i.v. administration of poorly water-soluble drugs by chemical derivatization to more water-soluble compounds. The most commonly used prodrug approach for increasing aqueous solubility of drugs containing a hydroxyl group is to produce esters containing an ionizable group; e.g., phosphate group, carboxylate group, alkylamino group (Fleisher et al., Advanced Drug Delivery

Reviews, 115-130, 1996; Davis et al., Cancer Res., 7247-7253, 2002, Golik et al., Bioorg. Med. Chem. Lett., 1837-1842, 1996).

In certain embodiments of the compounds of this invention, the prodrugs of the present invention are capable of serving as a substrate for a phosphatase, a carboxylase, or another enzyme

### Screening Assays for Compounds Having Utility for the Present Invention

According to additional aspects of the present invention, high-throughput assays are available to enable routine facile screening of compound libraries for compounds having utility for the present invention.

Inhibitors of the β-Catenin:CBP Interaction.

As an initial matter, various methods for identifying small molecule inhibitors of the β-catenin:CBP interaction are well described in the art and are, for example, discussed in detail in the patents and patent applications listed in Table 1, herein, and thus will not be repeated here.

### Primary Screens for Compounds Affecting Asymmetric Versus Symmetric Division in Stem Cells.

In vitro. Based on work that epidermal stem cells are heterogeneous in their capacity to be activated based on the status of their molecular circadian clock, an assay for screening activators of asymmetric division of this stem cell pool is utilized to identify compounds having utility for the present invention. More specifically, (Janisch P. et al. Nature 2011) demonstrated that the population of CD34 expressing bulge stem cells that express high levels of the genes Per1/2 are more likely to respond to activation and stimuli that remove them from dormancy. The transcription factor BMAL1, a member of the ARNt family of transcription factors in conjunction with its molecular partner clock drive the expression of a core of circadian genes including Per1 and Per2. Mice deficient in BMAL1 exhibit an early aging phenotype including premature aging of the skin.

Accordingly, a high throughput screen to select compounds that induce asymmetric division of epidermal stem cells, is provided by human keratinocytes transfected with a Per/luciferase reporter gene. Keratinocytes, that have been stably transfected with the human Per/luc promoter are grown in vitro, and then plated in either 96 or 384 well plates and screened with a chemical compound collection for compounds that increase luciferase expression. After treatment with compounds for 24 h, the cells will be lysed and treated with luciferase substrate and then read for luciferase activity on a high throughput plate reader (example HP Topcount). Promising compounds can be secondarily screened (e.g., see below).

### Secondary Screens for Compounds Affecting Asymmetric Versus Symmetric Division in Stem Cells.

Ex Vivo (human skin assay). Culture conditions and assay based on Varani J et al Experimental and Molecular Pathology, 2004.

According to further aspects, human skin (e.g., surgical waste from plastic surgery procedures) is obtained, and the subcutaneous layer of fat trimmed manually (e.g., with a scalpel). The skin is then cut into small fragments about 2 mm square and placed in 6 well plates. 1 ml of keratinocyte culture medium (Gibco 10724-011) with 1% P/S is added to each well. The epidermis, bathed in media is placed facing up. On the next day (overnight culture in the media above), the skin fragments are transferred to fresh wells that respectively contain the compounds to be tested. Approximately 24 hrs and 48 hrs skin samples in culture are removed for RNA isolation and qRT-PCR analysis of genes of interest (e.g., Per1, 2). Some skin fragments are transferred to wells with new medium every second day (and continued to be treated with compounds. Approximately 7 days after culturing ex vivo, Brdu, 20 uM final concentration per well is added to evaluate proliferation. On approximately the 9th day of culturing the skin ex vivo, the skin fragments are harvested for histology, immunohistochemistry (e.g., staining for Per1, 2) and BrdU staining to evaluate proliferation. Promising compounds can be subjected to tertiary screens (e.g., see below).

### Tertiary Screens for Compounds Affecting Asymmetric Versus Symmetric Division in Stem Cells.

In Vivo. Asymmetric cell divisions are important regulators of the stem cell niche. During this process, evolutionarily conserved sets of proteins (e.g., form C. elegans and Drosophila to humans) are asymmetrically distributed to daughter cells during mitosis. These include proteins of the Par complex e.g. Par3 in mammals (Bazooka in Drosophila), Par 6 and atypical Protein Kinase C (aPKC) as well as transcriptional regulators (e.g., numb, a negative regulator of Notch signaling). This process is also important in the control of asymmetric division in the epidermal stem cells niche (Williams S et al Nature 2011). According to particular aspects, therefore, in vivo assays can be to examine asymmetric distribution of these proteins during mitosis in the epidermal stem cell niche.

For example, Bultje et al. (Bultje R Neuron 63, 189-202, 2009) describe an assay to measure asymmetric distributions in the ventricular zone (vz) to evaluate asymmetric divisions during neurogenesis. Essentially this involves treating the animal (either adult or in utero) for a set period of time with compounds (either p.o., s.c., i.v. or topically) with compounds and then sacrificing the animal and examining the Par3 distribution (e.g., via immunohistochemistry) in mitotic cells vs. DNA distribution (e.g., using DAPI staining) Par3 distributes equally among the two daughter cells during symmetric division and unequally (essentially all in one daughter cell) during asymmetric differentiation. Applicant has utilized this assay to show that after topical or oral administration to pregnant mice, that the CBP/catenin antagonist ICG-001 does not affect the number of asymmetric divisions compared to vehicle control. However, the p300/catenin antagonist IQ-1, that increases CBP/catenin signaling at the expense of p300/catenin signaling decreases the number of asymmetric divisions and increases the number of symmetric divisions. Importantly, as discussed in more detail herein, treatment with excess ICG-001 corrects the defect in asymmetric divisions caused by IQ-1, confirming that re-equilibration of increased symmetric (i.e. CBP/catenin dependent) divisions can be corrected with a CBP/catenin antagonist like ICG-001.

R-spondin 1, as used herein, refers to R-spondin proteins having the claimed activity, including but not limited to human R-spondin 1 (hRspo1) (e.g., GenBank accession no. ABC54570.1 GI:84105054 (SEQ ID NO:2) encoded by cDNA (mRNA coding sequence) DQ318235.1 GI:84105053 (SEQ ID NO:1)), and including but not limited to Rspo1 sequence variant having the HRspo1 biological activity described herein (e.g., human variant 1 NP_{—}001033722.1 GI:84490388 (SEQ ID NO:4) encoded by cDNA (mRNA coding sequence) NM_{—}001038633.3 GI:339276003 (SEQ ID NO:3); human variant 2 NP_{—}001229837.1 GI:339276005 (SEQ ID NO:6) encoded by cDNA (mRNA coding sequence) NM_{—}001242908.1 GI:339276004 (SEQ ID NO:5); human variant 3 NP_{—}001229838.1 GI:339276007 (SEQ ID NO:8) encoded by cDNA (mRNA coding sequence) NM_{—}001242909.1 GI:339276006 (SEQ ID NO:7); and human variant 4 NP_{—}001229839.1 GI:339276103 (SEQ ID NO:10) encoded by cDNA (mRNA coding sequence) NM_{—}001242910.1 GI:339276102) (SEQ ID NO:9)). Preferred Rspo1 proteins are human Rspo1, including, for example, recombinant human Rspo1 expressed by viral expression vectors, and purified as described herein. In particular aspects, human Rspo1 having SEQ ID NO:12, encoded by cDNA SEQ ID NO:11 was used, which provides for a StrepII tag (ESAWSHPQFEK) at the c-terminal end of the Rspo1.

Specific aspects relate to biologically active R-spondin1 (R-spo1) polypeptides including, for example, biologically active variants, deletions, muteins, fusion proteins, and orthologs thereof (collectively R-spo1 proteins).

As appreciated in the art, human Rspo1 proteins are members of the R-spondin family and are characterized, inter alia, as comprising two cysteine-rich furin-like repeats/domains (e.g., amino acid positions 100-142 in SEQ ID NOS:2, 4, 6 and 10; amino acid positions 73-115 in SEQ ID NO:8; as presented in GenBank annotations in relation to the above exemplary Rspo1 accession numbers) followed by a thrombospondin type 1 domain, followed by a basic amino-acid-rich (BR) domain. Human R-Spondin1 shares 89%, 87%, 92%, 91%, 91%, and 89% amino acid identity with mouse, rat, equine, canine, caprine and bovine R-Spondin1, respectively.

### Variants of Rspo1 Nucleic Acids and Proteins

As used herein, a “biological activity” refers to a function of a polypeptide including but not limited to complexation, dimerization, multimerization, receptor-associated ligand binding and/or endocytosis, receptor-associated protease activity, phosphorylation, dephosphorylation, autophosphorylation, ability to form complexes with other molecules, ligand binding, catalytic or enzymatic activity, activation including auto-activation and activation of other polypeptides, inhibition or modulation of another molecule's function, stimulation or inhibition of signal transduction and/or cellular responses such as cell proliferation, migration, differentiation, and growth, degradation, membrane localization, and membrane binding. A biological activity can be assessed by assays described herein and by any suitable assays known to those of skill in the art, including, but not limited to in vitro assays, including cell-based assays, in vivo assays, including assays in animal models for particular diseases.

Preferably, Rspo1 biological activity, or “biologically active Rspo1” refers to the biological activity of Rspo1 proteins as a Wnt pathway activator/agonist, in combination with of a CBP/catenin antagonist, to protect, mitigate or otherwise treat radiation-induced depletion of the somatic stem cells for the at least one tissue compartment or type, as disclosed herein.

Preferably, the Rspo1, or variants thereof, comprise an amino acid sequence selected from the group consisting of SEQ ID NOS:2, 4, 6, 8, 10, 12, and biologically active SEQ ID NOS:2, 4, 6, 8, 10, 12 having, in each case, from 1, to about 3, to about 5, to about 10, or to about 20 conservative amino acid substitutions, or a biologically active fragment of any of these sequences. In particular aspects, Rspo1 protein, or variant thereof, comprises a sequence of SEQ ID NO:2, or SEQ ID NO:12 (which is a fusion protein having a c-terminal StrepII tag (ESAWSHPQFEK), or a biologically active, conservative amino acid substitution variant thereof having from 1, to about 3, to about 5, to about 10, or to about 20 conservative amino acid substitutions.

Functional Rspo1 protein variants are those proteins that display the biological activities of Rspo1 protein. Preferably, Functional Rspo1 protein variants are those that display the biological activity of Rspo1 proteins as a Wnt pathway activator/agonist, in combination with of a CBP/catenin antagonist, to protect, mitigate or otherwise treat radiation-induced depletion of the somatic stem cells for the at least one tissue compartment or type, as disclosed herein

As used herein, the term “wild type Rspo1 protein”, means a naturally occurring Rspo1 allele found which encodes a functional Rspo1 protein. Likewise, for the presently disclosed purposes, the term “mutant Rspo1 protein”, as used herein, refers to an Rspo1 protein, which encodes a functional Rspo1 protein, i.e. an Rspo1 protein allele encoding a functional Rspo1 protein, which, as used herein, refers to an Rspo1 protein having biological activity as disclosed herein. Mutant alleles of the Rspo1 protein-encoding nucleic acid sequences are designated as “Rspo1” herein. Mutant alleles can be either “natural mutant” alleles, which are mutant alleles found in nature (e.g., produced spontaneously without human application of mutagens) or induced mutant” alleles, which are induced by human intervention, e.g. by mutagenesis.

Variants of Rspo1 protein have utility for aspects of the present invention. Variants can be naturally or non-naturally occurring. Naturally occurring variants (e.g., polymorphisms) are found in various species and comprise amino acid sequences which are substantially identical to the amino acid sequence shown SEQ ID NOS:2, 4, 6, 8, 10, 12. Species homologs of the protein can be obtained using subgenomic polynucleotides of the invention, as described below, to make suitable probes or primers for screening cDNA expression libraries from other species, such as mice, monkeys, yeast, or bacteria, identifying cDNAs which encode homologs of the protein, and expressing the cDNAs as is known in the art. Orthologs are provided for herein.

Non-naturally occurring variants which retain substantially the same biological activities as naturally occurring protein variants are also included here. Preferably, naturally or non-naturally occurring variants have amino acid sequences which are at least 85%, 90%, 95%, 96%, 97%, 98%, 99% or greater than 99% identical to the respective amino acid sequences shown in SEQ ID NOS:2, 4, 6, 8, 10, 12. More preferably, the molecules are at least 98%, 99% or greater than 99% identical to the respective amino acid sequences shown in SEQ ID NOS:2, 4, 6, 8, 10, 12. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. 2:482-489, 1981.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§1.821-1.822, abbreviations for amino acid residues are shown in Table 2:

TABLE 2
Table of Correspondence

| SYMBOL | | |
|---|---|---|
| 1-Letter | 3-Letter | AMINO ACID |
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

It should be noted that all amino acid residue sequences represented herein by a formula have a left to right orientation in the conventional direction of amino-terminus to carboxyl-terminus. In addition, the phrase “amino acid residue” is defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

Guidance in determining which amino acid residues can be substituted, inserted, or deleted without abolishing biological or immunological activity can be found using computer programs well known in the art, such as DNASTAR software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids. Preferably, amino acid changes in the Rspo1 polypeptide variants are conservative amino acid changes, i.e., substitutions of similarly charged or uncharged amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting variant. Properties and functions of Rspo1 protein or polypeptide variants are of the same type as a protein comprising the amino acid sequence encoded by the nucleotide sequence shown in SEQ ID NOS:2, 4, 6, 8, 10, 12, although the properties and functions of variants can differ in degree.

Variants of the Rspo1 polypeptide disclosed herein include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do or do not affect functional activity of the proteins are also variants. Covalent variants can be prepared by linking functionalities to groups which are found in the amino acid chain or at the N- or C-terminal residue, as is known in the art.

A subset of mutants, called muteins, is a group of polypeptides in which neutral amino acids, such as serines, are substituted for cysteine residues which do not participate in disulfide bonds. These mutants may be stable over a broader temperature range than native secreted proteins (see, e.g., Mark et al., U.S. Pat. No. 4,959,314).

It will be recognized in the art that some amino acid sequences of the Rspo1 polypeptides of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of ligand binding to cell surface receptors (Ostade et al., Nature 361:266-268, 1993). Rspo1 polypeptides of the present invention may include one or more amino acid substitutions, deletions or additions, either from natural mutations or human manipulation.

Amino acids in the Rspo1 polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

As indicated, changes in particular aspects are preferably of a minor nature, such as conservative amino acid substitutions that do not significantly affect the folding or activity of the protein. Of course, the number of amino acid substitutions a skilled artisan would make depends on many factors, including those described above. Other embodiments comprise non-conservative substitutions. Generally speaking, the number of substitutions for any given Rspo1 polypeptide will not be more than 50, 40, 30, 25, 20, 15, 10, 5 or 3.

### Fusion Proteins

Fusion proteins comprising proteins or polypeptide fragments of Rspo1 polypeptide can also be constructed. Fusion proteins are useful for in various targeting, purification and assay systems. For example, fusion proteins can be used to identify proteins which interact with a Rspo1 polypeptide of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence can be used.

A fusion protein comprises two protein segments fused together by means of a peptide bond. Amino acid sequences for use in fusion proteins of the invention can be utilize the amino acid sequence shown in SEQ ID NOS:2, 4, 6, 8, 10, 12, or can be prepared from biologically active variants of SEQ ID NOS:2, 4, 6, 8, 10 or 12, such as those described above. The first protein segment can include of a full-length Rspo1 polypeptide. Other first protein segments can consist of about functional portions of SEQ ID NOS:2, 4, 6, 8, 10 or 12.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and virus protein fusions. Yet other fusions can include a StrepII tag (ESAWSHPQFEK) at the c-terminal end of the Rspo1 polypeptide.

These fusions can be made, for example, by covalently linking two protein segments or by standard procedures in the art of molecular biology. Recombinant DNA methods can be used to prepare fusion proteins, for example, by making a DNA construct which comprises a coding region for the protein sequence of SEQ ID NOS:2, 4, 6, 8, 10 or 12 in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

### Nucleic Acid Sequences Encoding Rsp01 Proteins and Variants, Muteins, Fusions, Etc., Thereof.

The present invention also contemplates the fabrication of DNA constructs (e.g., expression vectors, recombination vectors, etc.) comprising an isolated nucleic acid sequence containing the genetic element and/or coding sequence from the R-spo1 proteins operatively linked to gene expression control sequences. “DNA constructs” are defined herein to be constructed (not naturally-occurring) DNA molecules useful for introducing DNA into host cells, and the term includes chimeric genes, expression cassettes, and vectors.

As used herein “operatively linked” refers to the linking of DNA sequences (including the order of the sequences, the orientation of the sequences, and the relative spacing of the various sequences) in such a manner that the encoded protein is expressed. Methods of operatively linking expression control sequences to coding sequences are well known in the art. See, e.g., Maniatis et al., Molecular Cloning: A Laboratory Manual, Cold Spring Harbor, N. Y., 1982; and Sambrook et al., Molecular Cloning: A Laboratory Manual, Cold Spring Harbor, N. Y., 1989.

“Expression control sequences” are DNA sequences involved in any way in the control of transcription or translation. Suitable expression control sequences and methods of making and using them are well known in the art.

The expression control sequences preferably include a promoter. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds, Nucleic Acids Res., 15, 2343-2361, 1987. Also, the location of the promoter relative to the transcription start may be optimized. See, e.g., Roberts et al., Proc. Natl. Acad. Sci. USA, 76:760-764, 1979.

The promoter may include, or be modified to include, one or more enhancer elements. Preferably, the promoter will include a plurality of enhancer elements. Promoters containing enhancer elements provide for higher levels of transcription as compared to promoters that do not include them.

For efficient expression, the coding sequences are preferably also operatively linked to a 3′ untranslated sequence. The 3′ untranslated sequence will preferably include a transcription termination sequence and a polyadenylation sequence. The 3′ untranslated region can be obtained, for example, from the flanking regions of genes.

A 5′ untranslated leader sequence can also be optionally employed. The 5′ untranslated leader sequence is the portion of an mRNA that extends from the 5′ CAP site to the translation initiation codon.

Nucleic acid sequences comprising one or more nucleotide deletions, insertions or substitutions relative to the wild type nucleic acid sequences are another embodiment of the invention, as are fragments of such mutant nucleic acid molecules. Such mutant nucleic acid sequences (referred to as Rspo1 mutant sequences) can be generated and/or identified using various known methods, as described further below. Again, such nucleic acid molecules are provided both in endogenous form and in isolated form. In one embodiment, the mutation(s) result in one or more changes (deletions, insertions and/or substitutions) in the amino acid sequence of the encoded Rspo1 protein (i.e., it is not a “silent mutation”). In another embodiment, the mutation(s) in the nucleic acid sequence result in a modified (increased or decreased) biological activity of the encoded Rspo1 protein relative to the wild type protein.

The nucleic acid molecules may, thus, comprise one or more mutations, such as:

(a) a “missense mutation”, which is a change in the nucleic acid sequence that results in the substitution of an amino acid for another amino acid;

(b) a “nonsense mutation” or “STOP codon mutation”, which is a change in the nucleic acid sequence that results in the introduction of a premature STOP codon and thus the termination of translation (resulting in a truncated protein).

(c) an “insertion mutation” of one or more amino acids, due to one or more codons having been added in the coding sequence of the nucleic acid;

(d) a “deletion mutation” of one or more amino acids, due to one or more codons having been deleted in the coding sequence of the nucleic acid;

(e) a “frameshift mutation”, resulting in the nucleic acid sequence being translated in a different frame downstream of the mutation. A frameshift mutation can have various causes, such as the insertion, deletion or duplication of one or more nucleotides.

Thus in one embodiment, nucleic acid sequences comprising one or more of any of the types of mutations described above are provided. In another embodiment, Rspo1 sequences comprising one or more stop codon (nonsense) mutations, one or more missense mutations and/or one or more frameshift mutations are provided. Any of the above mutant nucleic acid sequences may be provided per se (in isolated form).

A nonsense mutation in an Rspo1 allele, as used herein, is a mutation in an Rspo1 allele whereby one or more translation stop codons are introduced into the coding DNA and the corresponding mRNA sequence of the corresponding wild type Rspo1 allele. Exemplary translation stop codons are TGA (UGA in the mRNA), TAA (UAA) and TAG (UAG). Thus, any mutation (deletion, insertion or substitution) that leads to the generation of an in-frame stop codon in the coding sequence will result in termination of translation and truncation of the amino acid chain. In one embodiment, a mutant Rspo1 allele comprising a nonsense mutation is an Rspo1 allele wherein an in-frame stop codon is introduced in the Rspo1 codon sequence by a single nucleotide substitution, such as the mutation of CAG to TAG, TGG to TAG, TGG to TGA, or CAA to TAA. In another embodiment, a mutant Rspo1 allele comprising a nonsense mutation is an Rspo1 allele wherein an in-frame stop codon is introduced in the Rspo1 codon sequence by double nucleotide substitutions, such as the mutation of CAG to TAA, TGG to TAA, or CGG to TAG or TGA. In yet another embodiment, a mutant Rspo1 allele comprising a nonsense mutation is an Rspo1 allele wherein an in-frame stop codon is introduced in the Rspo1 codon sequence by triple nucleotide substitutions, such as the mutation of CGG to TAA. The truncated protein lacks the amino acids encoded by the coding DNA downstream of the mutation (i.e. the C-terminal part of the Rspo1 protein) and maintains the amino acids encoded by the coding DNA upstream of the mutation (i.e. the N-terminal part of the Rspo1 protein).

A missense mutation in an Rspo1 allele, as used herein, is any mutation (deletion, insertion or substitution) in an Rspo1 allele whereby one or more codons are changed in the coding DNA and the corresponding mRNA sequence of the corresponding wild type Rspo1 allele, resulting in the substitution of one or more amino acids in the wild type Rspo1 protein for one or more other amino acids in the mutant Rspo1 protein.

A frameshift mutation in an Rspo1 allele, as used herein, is a mutation (deletion, insertion, duplication, and the like) in an Rspo1 allele that results in the nucleic acid sequence being translated in a different frame downstream of the mutation.

### EXAMPLES

### Example 1

### Sequential Treatment with AdRspo1+ICG-001 Mitigated Radiation Lethality and Rescued 70% (P<0.007) and 60% Mice (p<0.003), Irradiated with 9.4 Gy and 10.4 Gy Whole Body Irradiation (WBI), Respectively

Overview.

According to particular aspects, the Wnt-b-Catenin pathway critically regulates the intestinal homeostasis by regulating proliferation and differentiation of intestinal stem cells (ISC). Applicants previously demonstrated that adenovirus-mediated delivery of Rspondin1 (AdRspo1), a Wnt agonist, induces crypt ISC regeneration and protects C57BL/6 mice from lethality of 10.4 Gy whole body irradiation (16). At lower doses (≦9.6 Gy), AdRspo1 could mitigate radiation lethality. Note that serum levels of Rspo1 rises only about 24 hrs after injection of AdRspo1 (16).

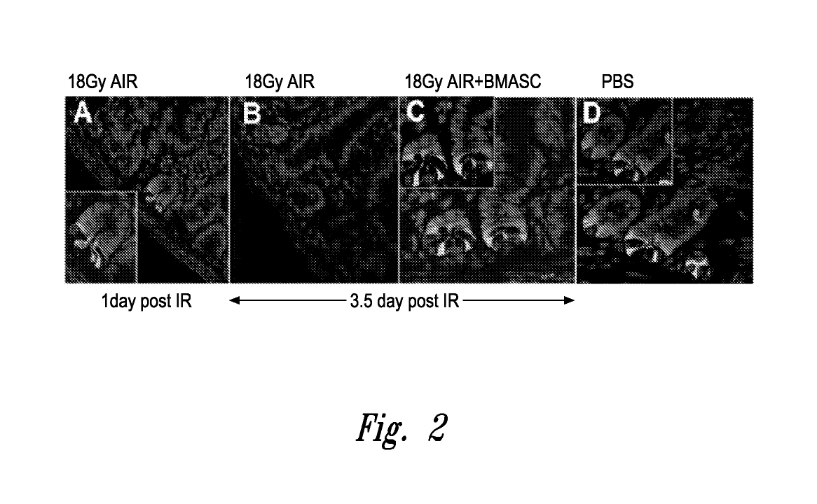

According to particular aspects, Lgr5+ve ISCs are still present up to 24-30 hrs after exposure to 18 Gy abdominal irradiation (see FIG. 2).

FIG. 2 shows, according to particular exemplary aspects, confocal microscopy demonstrating GFP expression in Lgr5-GFP transgenic mice. Note surviving Clonogens of Lgr5+ crypt base columnar cells (ISC) at 24 hr (A) but not at 3.5 days (B) following 18 Gy abdominal irradiation (AIR) (C) 3.5 days post-AIR and stromal cell transplantation and (D) Untreated PBS controls.

Within 3.5 days, these cells are invariably killed and animals fail to survive. According to particular aspects, this provides a window of opportunity for administration of intestinal growth factors that could rescue surviving ISC clonogens and stimulate repair and proliferation. While, growth factors alone fail to mitigate lethal doses of irradiation that induces RIGS, Applicants hypothesized that mitigation of RIGS would require induction of proliferation of residual ISCs, followed by, switching on the ISC differentiation in order to accelerate villi regeneration in irradiated mice.

This Example validates a novel method of treatment wherein sequential administration of an intestinal stem cell growth factor (e.g., Rspo1), followed by, a modulator (e.g., inhibitor) of β-catenin/TCF-mediated transcription (e.g., ICG-001), 2-4 days post-irradiation induced differentiation and accelerated intestinal regeneration for RIGS mitigation.

Methods.

C57BL/6 mice were treated with recombinant adenovirus expressing human Rspo1, AdRspo1 (5×10⁹ particles/mice, 1 hr and 48 hr post-IR), followed by ICG-001 (150 mg/kg of body weight, 72 hr post-IR) after whole body irradiation (WBI) of 9.4-10.4 Gy. Animals were observed for survival (Kaplan-Meier) and histopathological analysis (hematoxylin-eosin staining, TUNEL and Ki67 immunohistochemistry). Expression of mRNA levels of β-Catenin target genes in crypt cells was determined by qRT-PCR.

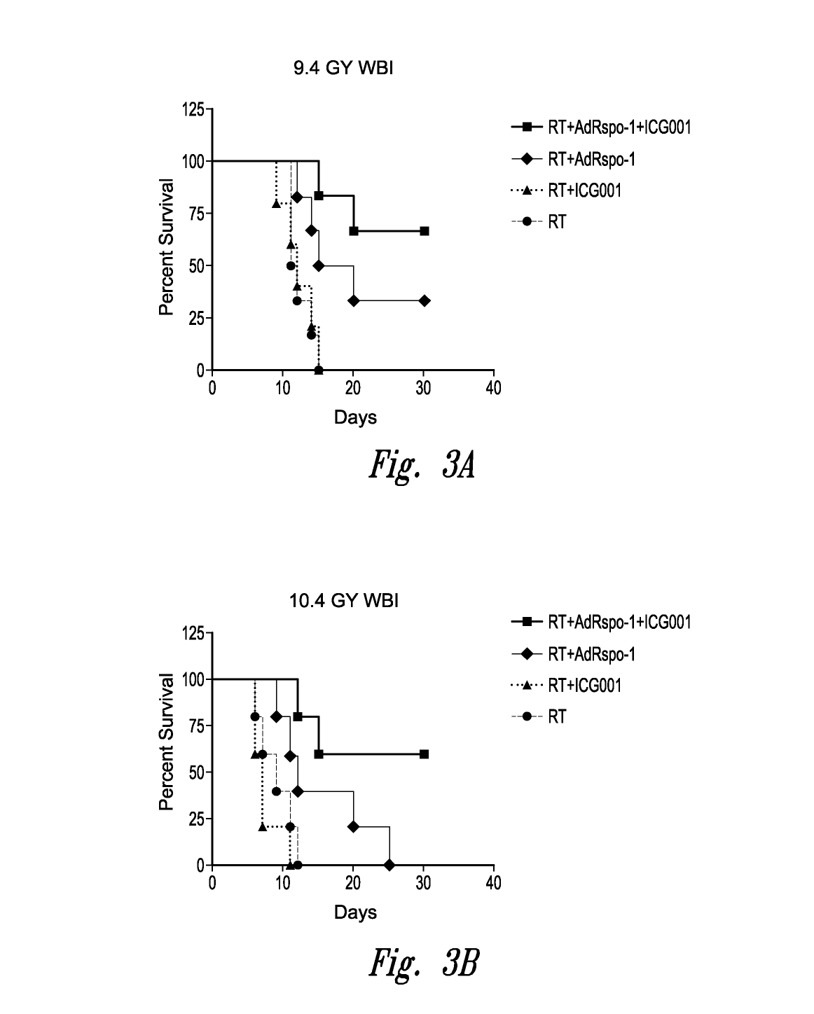

In vivo intestinal stem cell (ISC) transplantation and lineage tracing assay. In addition to Applicants' in vitro clonogenic assay, an ISC transplantation assay has been developed. Intestinal organoid suspensions in HGF-supplemented matrigels were implanted in subcutaneous fat pad of C57BL/6 mice, using protocols described before. ISC proliferation and differentiation were examined by histological and immunohistochemical examination (FIG. 3B).

Results.

As shown in FIG. 3, sequential treatment with AdRspo1+ICG-001 mitigates radiation lethality and rescued 70% (P<0.007) and 60% mice (p<0.003), irradiated with 9.4 Gy and 10.4 Gy whole body irradiation (WBI), respectively. Control mice receiving supportive care died within 10-15 days after exposure to 9.4-10.4 Gy WBI.

FIG. 3 shows, according to particular exemplary aspects, that systemic administration of ICG-001 plus AdRspo1, post-radiation exposure, mitigates RIGS. Treatment with AdRspo-1 followed by ICG-001, 72 hrs after irradiation, mitigates radiation lethality and rescued 70% mice (P<0.007) irradiated with 9.4 Gy and 60% mice (p<0.003) exposed to 10.4 Gy. All the untreated control mice died within 10-15 days after exposure to 9.4-10.4 Gy WBI.

### Example 2

### Sequential Treatment with AdRspo1+ICG-001 Increased Crypt Cell Proliferation, Crypt Depth and Villi Thickness, Decreased Crypt Cell Apoptosis, and Resulted in Marked Increases in Brachury T (244+8.9 Fold) and cJun (170+6.3 Fold), Resulting in Accelerated Regeneration and Improved Survival, Thus Mitigating RIGs

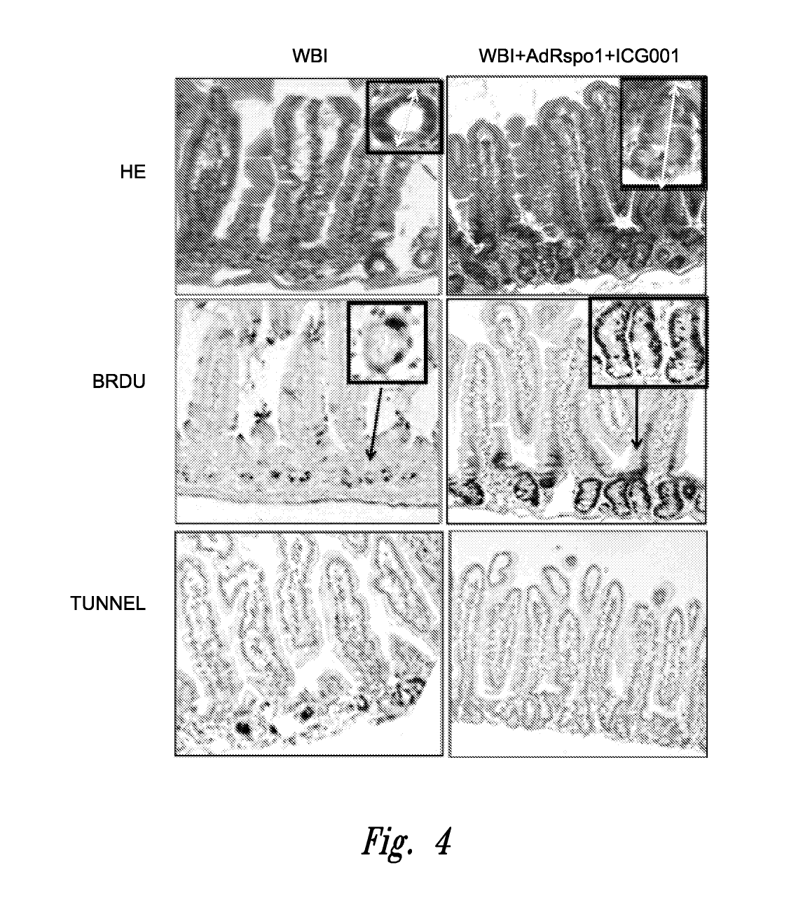

According to particular aspects, compared to control whole body irradiated (WBI) animals, sequential AdRspo-1+ICG001 treatment increased the crypt depth and villi thickness (see FIG. 4: hematoxylin and eosin (HE) staining)

BRdU immonohistochemistry of intestinal sections demonstrated increased crypt cell proliferation (incorporation of BRdU in newly synthesizing DNA of proliferating cells) and Tunnel staining showed a decrease in apoptosis in intestinal crypt cells in AdRspo1 and ICG001 treated animals, post-WBI (FIG. 4).

FIG. 4 shows, according to particular exemplary aspects, a histopathological assessment of intestine after 10.4 Gy whole body Irradiation. Histopathological evaluation of jejunum demonstrated larger crypt depth, intact villi (Hematoxylin-Eosin, HE), increased BrdU uptake in crypt and reduced apoptosis (TUNNEL) in AdRspo-1+ICG-001-treated animals, compared to irradiated controls (WBI), indicating structural regeneration of the irradiated intestine.

Quantitative RT-PCR analysis of genes associated with ISC differentiation showed a marked increase in Brachury T (244+8.9 fold) and cJun (170+6.3 fold) in WBI+AdRspo1+ICG001-treated animals, compared to WBI controls (see Table 2).

TABLE 2
qRT-PCR analysis of genes associated with ISC differentiation.

| Name | WBI + AdRspo1 + ICG-001 vs WBI |
|---|---|
| of the genes | (fold increase) |
| Brachury T | 244 ± 8.9 |
| c-Jun | 170 ± 6.3 |
| Foxn1 | 36.29 ± 2.1 |
| Ctbp2 | 15.48 ± 2.46 |
| SOX17 | 2.56 ± 0.56 |

According to particular aspects, Applicants results show that intestinal stem cell differentiation is crucial for intestinal regeneration after lethal radiation exposure. Post-radiation exposure, sequential treatment with Rspo-1 and ICG-001 modulates the Wnt-β-Catenin pathway in intestine, resulting in accelerated regeneration and improved survival.

According to particular aspects, therefore, a sequential combination of intestinal stem cell growth factor (Rspo1) and a differentiating agent (ICG-001) is substantially effective in mitigating RIGS.

### Example 3

### Optimization of Sequential Treatment with AdRspo1+ICG-001 for Mitigating RIGs

According to particular aspects, experiments are performed in irradiated and control mice to optimize the timing of drug delivery, R-spo 1 (2, 6, 12 and 24 hrs post-IR) and ICG-001 (24, 36, 48 and 72 hrs post-IR), dose and route of delivery (subcutaneous versus intravenous).

According to particular aspects, experiments are performed in irradiated and control mice to optimize the timing of drug delivery, R-spo1 (24 hrs post-IR) and ICG-001 (24, 36, 48 and 72 hrs post-IR), dose and route of delivery (subcutaneous versus intravenous).

The extent of radio-mitigation by ICG-001 in mice is investigated after exposure to various doses of whole body (6-12 Gy single fraction) and abdominal irradiation (14-18 Gy).

All animal experiments are followed according to the ongoing U-19 grant and IACUC approval.

Toxicity of ICG-001 is assessed by administering the drug in nave mice and blood and tissues are collected for evaluation of organs, such as, bone marrow, liver, kidney, lung and intestine function. Body weight is measured.

According to particular exemplary aspects, post-IR treatment with the agonist of Wnt-β-catenin signaling (e.g., R-sp01) precedes post-IR treatment with the CBP/β-catenin inhibitor (e.g., ICG-001).

According to particular exemplary aspects, the agonist of Wnt-β-catenin signaling (e.g., R-sp01) is administered at 2 hrs, 6 hrs, 12 hrs or at 24 hrs post-IR.

According to particular exemplary aspects, the CBP/β-catenin inhibitor (e.g., ICG-001) is administered at 24 hrs, 36 hrs, 48 hrs or at 72 hrs post-IR.

According to particular aspects, administration of recombinant Rspo1 (e.g., within 24 hrs post-exposure) rescues surviving ISC clonogens and therefore, is effective in radio-mitigation.

According to particular aspects, the radiation mitigation properties ICG-001 depend upon the time of drug administration. Ideally, the residual ISC clonogens in irradiated intestine is amplified before differentiation is induced, and thus the sequential timing of administration of ICG-01 is critical for successful mitigation of RIGS.

According to particular aspects, the timing of administration of ICG-001 after irradiation exposure is sufficient to allow enough time for proliferation of surviving ISC clonogens.

In particular exemplary aspects, ICG-001 is effective in mitigating RIGS after 72 hrs of irradiation exposure. As will be recognized by one of skill in the art, the sequential timing of ICG-001 administration relative to irradiation exposure and/or administration of the agonist of Wnt-β-catenin signaling can be readily determined using that assays described herein without undue experimentation.

### Example 4

### An In Vitro Assay Model Using the Lgr5+Ve Crypt ISCs, Isolated from Lgr5EGFPires CreERT2 Mice Crossed with the Cre-Activatable Rosa26LacZ Reporter, is Used to Elucidate the Mechanisms of Diomitigation by ICG-001 in RIGs

Overview.

Intestinal homeostasis is critically regulated by self-renewal/proliferation and differentiation of ISCs. In response to radiation injury, maintenance of a balance between proliferation and differentiation could be the crucial for structural regeneration.

An in vitro assay model using the Lgr5+ve (leucine-rich-repeat-containing G-protein-coupled receptor 5, also known as Gpr49) crypt ISCs, isolated from 1-gr5EGFPiresCreERT2 mice crossed with the Cre-activatable Rosa26LacZ reporter (17) is used to allow lineage tracing as a signature of differentiation after induction with low dose tamoxifen to activate cre. Cre-mediated excision of the roadblock sequence in the Rosa26-lacZ reporter irreversibly marks Lgr5+ cells. Moreover, although potential progeny of these cells no longer express GFP, the activated lacZ reporter acts as a genetic marker, facilitating lineage tracing. Therefore, formation of crypt like structure in culture with prevalence of blue cells (X-gal staining) determines the regeneration along with lineage tracing.

Experiments are performed to determine whether ICG-001 augments differentiation of ISC in irradiated and control mice. An in vitro and in vivo intestinal crypt cell clonogenic assay is performed to examine the dose response of Rspo1/ICG-001-mediated crypt cell regeneration and differentiation after exposure to irradiation. The effects of ICG-001 on cell cycle distribution of intestinal crypt cells are studied.

In vitro Clonogenic and differentiation assay. Isolated small intestines are opened. longitudinally, and washed with cold PBS. The tissue is chopped into around 5 mm pieces, and further washed with cold PBS. The tissue fragments are incubated in 2 mM EDTA with PBS for 30 min on ice. After removal of EDTA medium, the tissue fragments are vigorously suspended by using a 10-ml pipette with cold PBS. The sediment is resuspended with PBS. After further vigorous suspension and centrifugation, the supernatant enriched with crypts is passed through a 70-mm cell strainer (BD Bioscience, San Jose, Calif.) to remove residual villous material. isolated crypts are incubated in culture medium for 45 min at 37° C., followed by trituration with a glass pipette to dissociate in a single cell suspension. Cells are sorted by flow cytometry (MoFlo; Dako) on the basis of GFP+ve LGR5 cells. Sorted GFPhi cells are collected in crypt culture medium and embedded in Matrigel prior to plating. After exposure to graded doses of irradiation (IR) (2-8 Gy) cells are treated sequentially with Rspondin-1 (1-6 hr Post IR) and ICG001 (2-7 hr Post IR) followed by cre induction with Tamoxifin. After 4-6 days of culture, the number of surviving organoids containing blue cells is counted. Organoids are further stained with villin (enterocytes), Muc2 (goblet cells), lysozyme (Paneth cells) and chromogranin A (enteroendocrine cells) to determine all four mature cell types.

For in vitro culture, most of the sorted cells may die within first 12 h of culture presumably as a result of physical and/or biological stress inherent in the isolation procedure and/or flowcytometry. Moreover, maintenance of a prolonged culture of sorted cells may require stromal support rather than matrigel, especially after exposure to radiation. To minimize the stress from flow cytometric sorting the cells are plated directly after isolation. It is expected that differentiated mature cells will die within 12-24 hr and stem/progenitor cells will remain. Cells are allowed to stay in culture for 24-36 hr for preconditioning prior to radiation. To develop a physical and biochemical support resembling the stromal niche for in vitro culture of intestinal stem cell a three-dimensional intestinal culture system is used according to the protocol described by Ootani et al (18). Unlike matrigel this system allows myofibroblasts and the collagen matrix to stay in close proximity to ISCs and therefore serve as a niche. In brief, the mouse small intestines are opened and washed in PBS to remove all luminal contents. Tissue is minced a 1-cm segment on ice with iris scissors and embedded in a 3D collagen gel using a double-dish culture system. 1-ml collagen gel solution (Cellmatrix Type I-A, Nitta Gelatin) is added into a 30-mm dish (Millicell-CM, Millipore), the inner dish, with a hydrophilic polytetrafluomethy ene membrane bottom to form an acellular layer. Next, a 1-int collagen gel solution containing a total of 0.1 g minced tissues is placed on the acellular layer in the dish. This inner dish is placed into a 60-mm outer dish containing 1.5 ml Ham's F12 medium supplemented with 20% FCS and 50 μg ml-1 gentamicin (Gibco). The culture is carried out in 37° C. in a humidified atmosphere of 5% CO2 in air and will be monitored for intestinal spheres. Spheres are subjected X-gal staining for lineage tracing.

In vivo intestinal stem cell (ISC) transplantation and lineage tracing assay. In addition to Applicants' in vitro clonogenic assay, an ISC transplantation assay has been developed. Intestinal organoid suspensions in HGF-supplemented matrigels are implanted in subcutaneous fat pad of C57BL/6 mice, using protocols described before. ISC proliferation and differentiation are examined by histological and immunohistochemical examination. Various intestinal epithelial cell types with attention to ISC differentiation into intestinal mucosa with full crypt-villus architecture are identified and used to examine growth and differentiation of ISC.

As noted herein, experiments are performed in irradiated and control mice to optimize the timing of drug delivery, R-spo1 (2, 6, 12 and 24 hrs post-IR) and ICG-001 (24, 36, 48 and 72 hrs post-IR), dose and route of delivery (subcutaneous versus intravenous). For lineage tracing assay, a transgenic mice with knock-in allele in which Lgr5 followed by LacZ reporter gene under Cre-lox promoter (Jackson laboratory, Maine) is used to trace the Lgr5 (ISC specific marker) expressing ribbon of blue cells extending from a single CBC cell at the base of the crypt to the up of the villi, thus suggesting Lgr5+CBCs are multipotent and capable of generating entire villus epithelium with all epithelial cell types present in the blue ribbon (17). After irradiation, cre are induced by tamoxifen injection. This mouse model is used to study the effect of irradiation on pluripotency of ISC upon irradiation in vivo along with the effect of Rspo1+ICG-001. Differentiation is noted by immunohistochemistry with villin (enterocytes), Muc2 (goblet cells), lysozyme (Paneth cells) and chromogranin A (enteroendocrine cells) to determine all four mature cell types.

### Example 5

### Small Molecule Induces of Rspo1 can be Used to in Place of Rspo1 for Maintenance of Small Intestinal Crypt Organoid Cultures Ex Vivo

Overview.

This working example confirms that small molecule induces of Rspo1 can be used to replace Rspo1 for maintenance of small intestinal crypt organoid cultures ex vivo.

Methods.

Small intestinal crypt organoid culture was performed using murine intestinal crypts essentially as described by Ootani et al (Nature Medicine; online publication doi:10.1038/nm.1951, 27 Apr. 2009)), and see also Sato et al (Nature Letters doi:10.1038/nature07935, 2009), both incorporated by reference herein in their entireties.

Results.

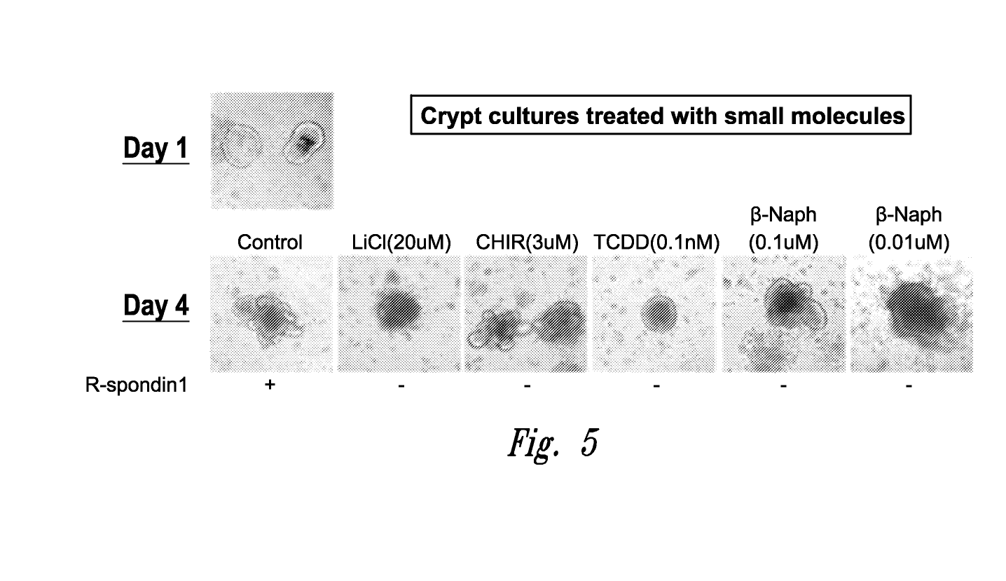

FIG. 5 shows that small molecule induces of Rspo1 (e.g., either direct Wnt/catenin activators such as LiCl, or GSK3beta inhibitors such as CHIR, or arylohydrdocarbon receptor (AHR) agonists such as beta napthoflavone, or indole-3-carbinol (I3C, which under the influence of stomach acids can be converted to the high-affinity AhR ligands DIM and ICZ), or formylindolo[3,2-b]carbazolsin particular 6-formylindolo[3,2-b]carbazole (FICZ) FICZ-derived indolo[3,2-b]carbazole-6-carboxylic acid metabolites and sulfoconjugates, which induce the expression of Rspo1) can be used to replace (for at least up to 4 days) Rspo1 for maintenance of small intestinal crypt organoid cultures ex vivo. N.B. in FIG. 5 means half dose of Rspo1.

### Example 6

### Human Recombinant R-Spondin1 (Rspo1) was Expressed and Produced Using Viral Transduction of HEK 293 Cells, Cell Sorting and Column Chromatography

Overview.

This working example describes expression and production of purified human recombinant Rspo1 (SEQ ID NO:12; with StrepII tag (ESAWSHPQFEK) at the c-terminal end), using HEK 293 cells transduced with lentiviral expression vectors expressing human Rspo1, followed by cell sorting and column chromatography.

### Methods.

Construct Design.

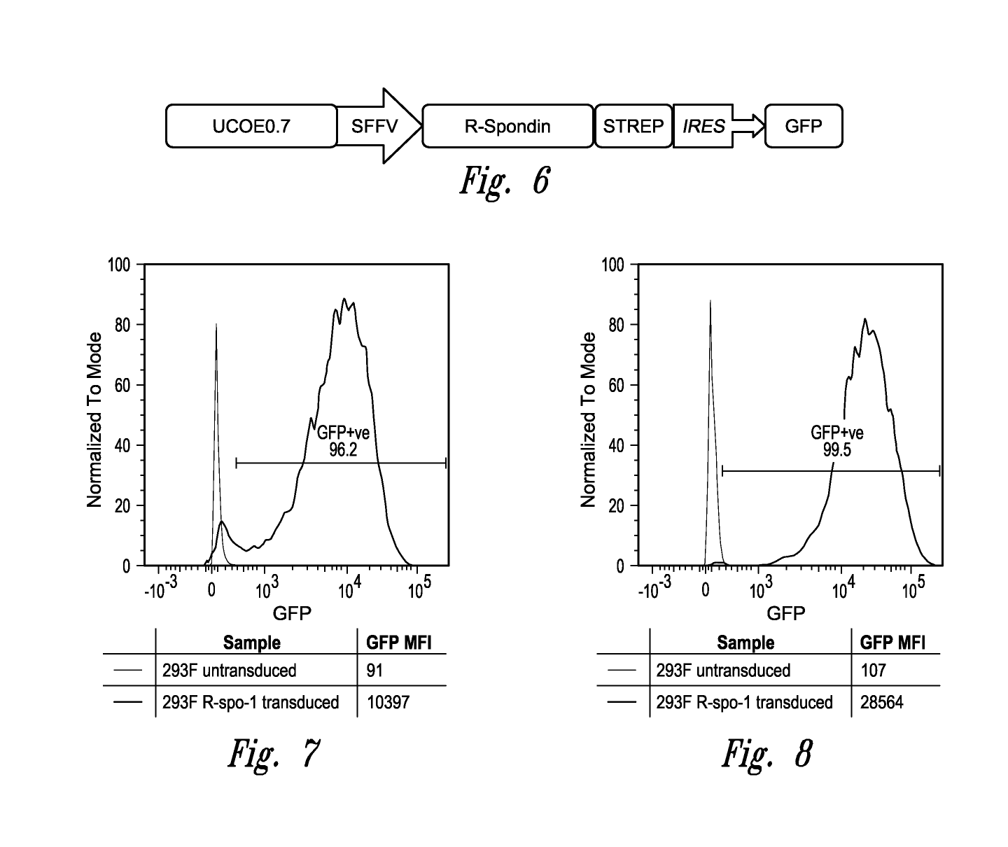

Human Rspo1 was cloned into the Daedalus lenti-viral vector illustrated in FIG. 6. Inherent limitations to traditional lentiviral systems include constrained packaging size (lentiviral particles are capable of packaging only about 10 kilobases (kb) of DNA efficiently (inclusive of viral DNA) and genomic silencing of integrated lentiviral transgenes, seen both in vitro and in vivo. The Daedalus system uses an optimized lentiviral expression vector that contains a novel and minimized 0.7 kb Ubiquitous Chromatin Opening Element (UCOE0.7) fragment of the HNRPA2B1/CBX3 locus. This combination allows for the enhanced expression of recombinant proteins with sizes approaching 70 kDa. In addition, Daedalus utilizes a cis-linked fluorescent reporter (GFP) driven by an internal ribosome entry site (IRES) that allows for rapid detection of transduced populations, tracking relative protein expression levels and facilitates isolation of high expressing clones by FACS sorting. This construct also affords a C-terminal STREP II tag for purification. The recombinant cDNA used was SEQ ID NO:11, encoding for recombinant human Rspo1 (SEQ ID NO:12, which StrepII tag).

Viral Transduction of 293F Cells.

HEK 293 Freestyle (293F) cells from Invitrogen are suspension and serum free adapted human cells and are, as a result, ideal for the production of mammalian secreted proteins. R-spo 1 virus was used to transduce 293F cells at an MOI ˜10. Transduction efficiency was analyzed by FLOW cytometry using the IRES driven GFP reporter and was greater than 90% (FIG. 7). FIG. 7 shows FLOW cytometry analysis of Lenti-R-spo 1 transduction of 293F cells; histogram shows successful (>95%) transduction of 293F cells using the R-spo 1 virus.

FLOW Cytometry Based Sorting of High Expressing Rspo1 Population.

In order to obtain the highest expressing population, the R-spo 1 transduced cells were sorted based on GFP (FIG. 8). FIG. 8, shows, according to particular exemplary aspects, FLOW cytometry analysis of Lenti-R-spo 1 sorted population; histogram shows that the sorted population is approximately 100% GFP positive and has an almost 3-fold increase in GFP mean fluorescence intensity as compared to the pre-sort population in FIG. 7. The highest 10% of GFP positive cells were sorted and expanded for protein production.

Purification of Rspo1.

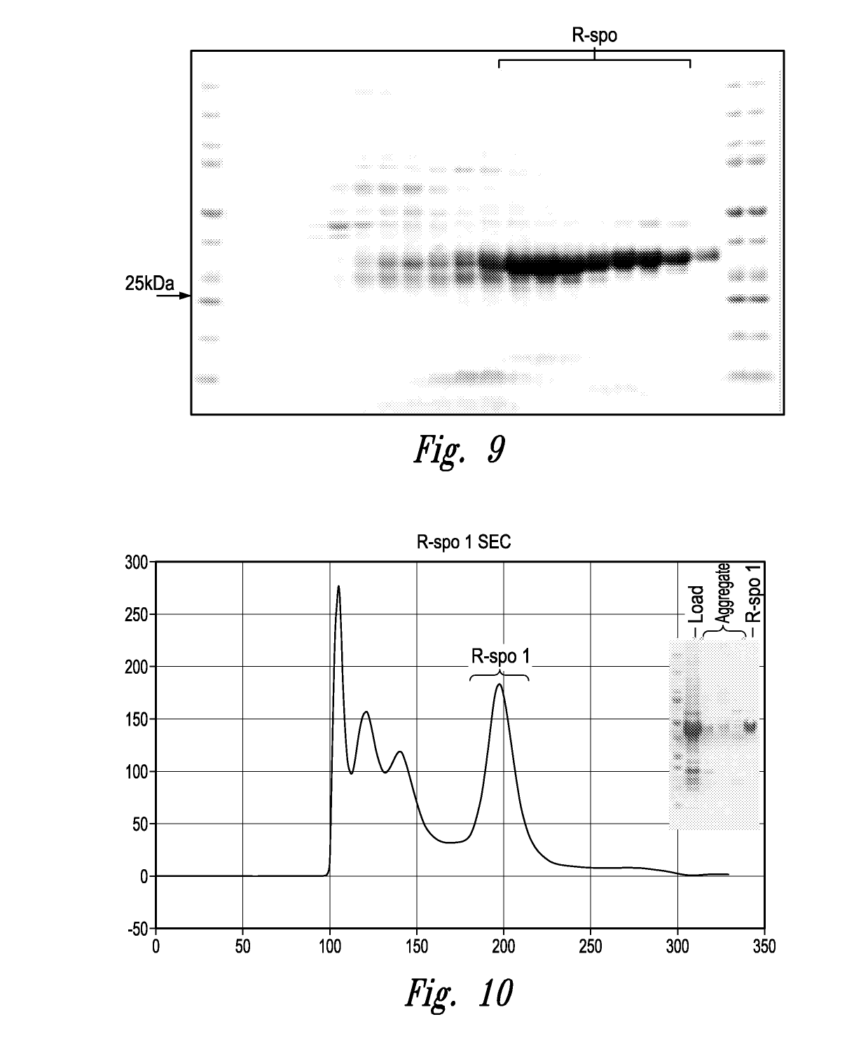

The Rspo1 sorted population (see FIG. 3) was scaled up to a final volume of 2 L for purification, which took approximately 10 days of culture. At the end of the incubation, the supernatant was harvested by centrifugation and the protein was first purified using a Heparin column. Briefly, a 5 ml Heparin Hi-Trap™ column (GE Healthcare) was equilibrated with PBS and the supernatant was loaded onto the column at a flow rate of 2 ml/min. Once loaded, the target protein was eluted using a sodium chloride gradient (FIG. 9). FIG. 9 shows, according to particular exemplary aspects, the results of running heparin purification R-spo 1 fractions on an SDS gel.

The pooled fractions from the heparin purification was then concentrated to approximately 5 ml and applied to a size exclusion chromatography (SEC) column equilibrated with PBS and 10% glycerol for final polishing (see FIG. 10). The final yield of R-spo 1 was approximately 10 mg/L from 2 liters, which was calculated from the pooled fractions by UV absorbance at 280 nm and with an extinction coefficient of 0.8. The identity of the purified protein was confirmed by mass spectroscopy. FIG. 10 shows, according to particular exemplary aspects, a UV trace of R-spo 1 size exclusion purification; the FIG. 10 insert shows SDS PAGE gel analysis of fractions corresponding to the aggregate peaks and R-spo 1.

Biological activity of the purified recombinant human R-spo 1 (SEQ ID NO:12) was confirmed as shown in working Examples 7 and 8 below.

### Example 7

### The Biological Activity of Purified Human Recombinant R-Spondin1 (Rspo1) was Confirmed in 293T Cells, and Also Ex Vivo Using In Vitro Intestinal Organoid Culture

Functional Assay of Recombinant Human Rspo1.

The recombinant cDNA used was SEQ ID NO:11, encoding for recombinant human Rspo1 (SEQ ID NO:12, with StrepII tag). Activity of the purified recombinant Rspo1 was assayed using a β-catenin activation test.

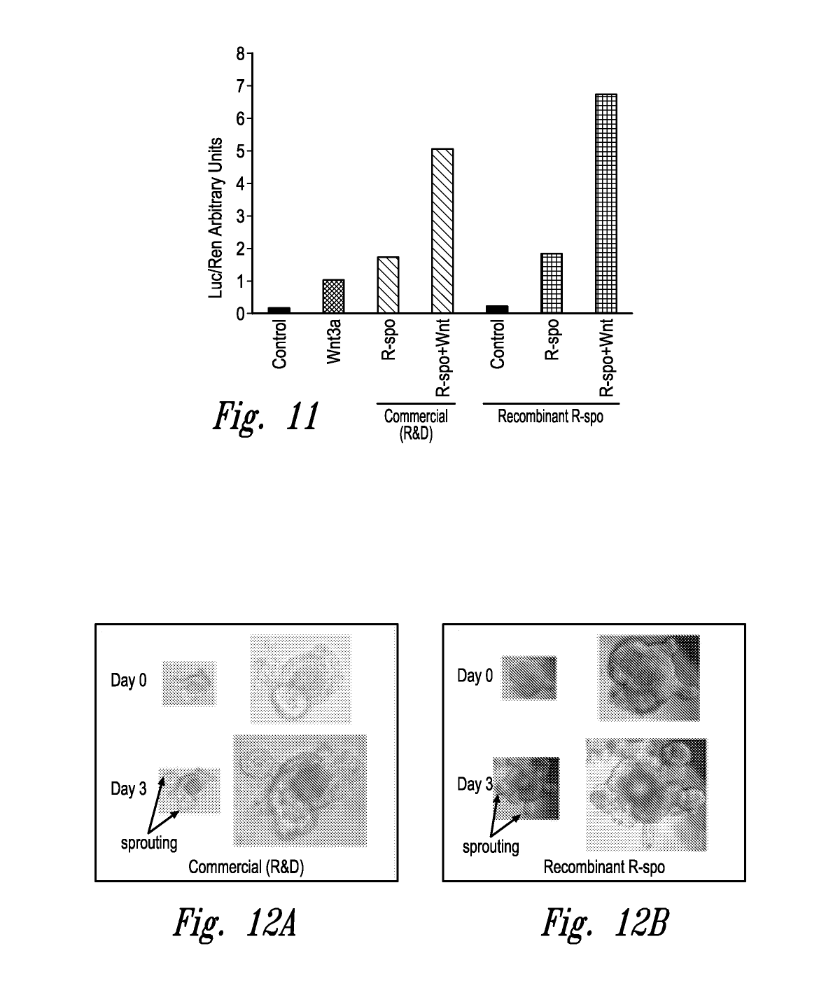

Briefly, 293T cells were transfected with the reporter vector TopFlash, along with TK Renilla (transfection control) using lipofectamine 2000. After the transfection mixture was removed, cells were incubated in 0.1% FCS o/n, and then stimulated with the different inducers at the indicated concentrations in 1% FCS for 24 hours (FIG. 11).

FIG. 11 shows, according to particular exemplary aspects, a Top Flash Luciferase assay demonstrating that recombinant R-spo 1 activates the Wnt pathway in 293 cells. FIG. 11 shows that the recombinant R-spo 1 is capable of activating β-catenin by itself and synergizing with Wnt (e.g., Wnt3A) as well as, if not better, than the commercially obtained Rspo1 from R&D biosystems.

The recombinant human Rspo1 was also tested in an intestinal organoid growth/maintenance test. Self-renewal of the small intestinal and colonic epithelium is driven by the proliferation of stem cells and their progenitors; and their maintenance and growth in culture (ex-vivo) requires the activation of the Wnt pathway. Given that it is essential, only functional Rspo1 will be able to maintain the growth of intestinal organoids and lead to their maintenance and proliferation. FIGS. 12A and 12B show, according to particular exemplary aspects, testing of functionality of recombinant human Rspo1 in an intestinal organoid growth/maintenance assay. The recombinant human protein was capable of maintaining the growth and proliferation of mouse intestinal organoids in culture.

Organoids were generated by culturing purified small intestinal crypts in matrigel in Advanced DMEM/F12 supplemented with 500 ng/ml R-spondin 1 [commercial (FIG. 12A) or recombinant (FIG. 12B)], 100 ng/ml EGF and 100 ng/ml Noggin. The ability of the recombinant R-spo to maintain and induce sprouting (proliferation) of the organoids was identical to the commercial protein suggesting that it is fully functional.

### Example 8

### Sequential Administration of Recombinant Human Rspo1 (hRspo1) and ICG-001 Resulted in Mitigating RIGS In Vivo in Irradiated Mice

Overview.

This working Example shows that sequential administration of recombinant human Rspo1 (hRspo1) and a novel Intestinal stem cell differentiation agent, ICG-001 mitigated RIGS. The recombinant cDNA used was SEQ ID NO:11, encoding for recombinant human Rspo1 (SEQ ID NO:12, with StrepII tag).

Other studies disclosed herein above demonstrated that sequential administration of a recombinant adenovirus expressing human Rspo1 followed by ICG-001 mitigated Radiation Induced Gastrointestinal Syndrome (RIGS), and resulted in improved survival in mice.

To examine whether the purified recombinant hRspo1 could mitigate RIGS when administered along with ICG001, we treated C57BL/6 mice after 10.4 Gy of WBI with the combination of recombinant hRspo1+ICG-001. Specifically, C57BL/6 mice (n=10), exposed to 10.4 Gy single fraction WBI, were treated with hRspo1 (20 mg/kg of body weight s.c.) at 24 hr and 48 hr post-WBI. A separate cohort received sequential treatment of hRspo1 (24 hr and 48 hr post WBI) followed by ICG-001 (150 mg/kg of body weight, 72 hr post-IR).

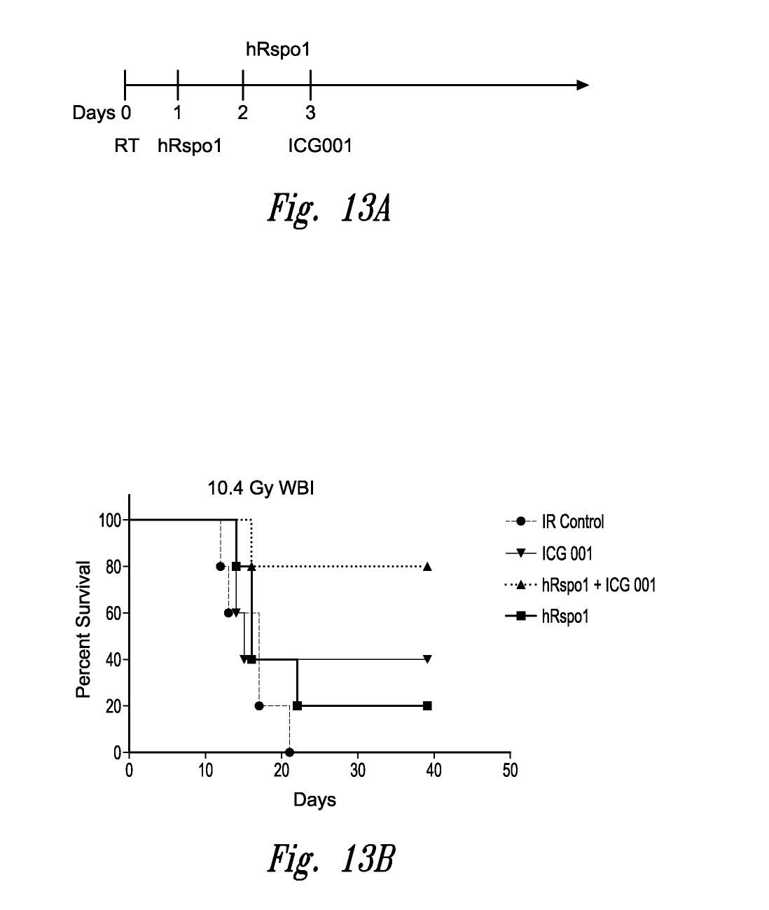

As shown in FIGS. 13A and 13B, treatment with hRspo1+ICG-001 mitigates radiation lethality and rescued 80% (P<0.001) of the mice exposed to 10.4 Gy whole body irradiation (WBI). Mice receiving only supportive care died within 10-15 days after exposure to 10.4 Gy WBI.

Thus, according to particular aspects, a sequential combination of intestinal stem cell growth factor (e.g., hRspo1) and a differentiating agent (e.g., ICG-001) act to mitigate RIGS.

### REFERENCES; INCORPORATED HEREIN BY REFERENCE IN THEIR ENTIRETIES

1. Hanson W R, Thomas C. 16,16-dimethyl prostaglandin E2 increases survival of murine intestinal stem cells when given before photon radiation. Radiat Res 1983; 96: 393-8.
2. Khan W B, Shui C, Ning S, Knox S J. Enhancement of murine intestinal stem cell survival after irradiation by keratinocyte growth factor. Radiat Res 1997; 148: 248-53.
3. Potten C S. Interleukin-11 protects the clonogenic stem cells in murine small-intestinal crypts from impairment of their reproductive capacity by radiation. Int J Cancer 1995; 62: 356-61.
4. Potten C S, Owen G, Hewitt D, et al. Stimulation and inhibition of proliferation in the small intestinal crypts of the mouse after in vivo administration of growth factors. Gut 1995; 36: 864-73.
5. Inagaki-Ohara K, Yada S, Takamura N, et al. p53-dependent radiation-induced crypt intestinal epithelial cells apoptosis is mediated in part through TNF-TNFR1 system. Oncogene 2001; 20: 812-8.
6. Martin K, Potten C S, Kirkwood T B. Age-related changes in irradiation-induced apoptosis and expression of p21 and p53 in crypt stem cells of murine intestine Ann N Y Acad Sci 2000; 908: 315-8.
7. Matsuu M, Shichijo K, Okaichi K, et al. The protective effect of fermented milk kefir on radiation-induced apoptosis in colonic crypt cells of rats. J Radiat Res (Tokyo) 2003; 44: 111-5.
8. Park Y J, Fujisaki S, Kimizuka K, et al. Apoptosis of crypt cells and lymphocytes in gut-associated lymphoid tissues during small intestinal graft rejection in rats. Transplant Proc 2004; 36: 353-5.
9. Kim K A, Wagle M, Tran K, et al. R-Spondin Family Members Regulate the Wnt Pathway by a Common Mechanism. Mol Biol Cell 2008.
10. Nam J S, Turcotte T J, Smith P F, Choi S, Yoon J K. Mouse cristin/R-spondin family proteins are novel ligands for the Frizzled 8 and LRP6 receptors and activate beta-catenin-dependent gene expression. J Biol Chem 2006; 281: 13247-57.
11. Kim K A, Kakitani M, Zhao J, et al. Mitogenic influence of human R-spondin1 on the intestinal epithelium. Science 2005; 309: 1256-9.
12. Li G, Shinozuka J, Uetsuka K, Nakayama H, Doi K. T-2 toxin-induced apoptosis in intestinal crypt epithelial cells of mice. Exp Toxicol Pathol 1997; 49: 447-50.
13. Pinto D, Gregorieff A, Begthel H, Clevers H. Canonical Wnt signals are essential for homeostasis of the intestinal epithelium. Genes Dev 2003; 17: 1709-13.
14. Eguchi M, Nguyen C, Lee S C, Kahn M. ICG-001, a novel small molecule regulator of TCF/beta-catenin transcription. Med Chem 2005; 1: 467-72.
15. Emami K H, Nguyen C, Ma H, et al. A small molecule inhibitor of beta-catenin/CREB-binding protein transcription [corrected]. Proc Natl Acad Sci USA 2004; 101: 12682-7.
16. Bhanja P, Saha S, Kabarriti R, et al. Protective role of R-spondin1, an intestinal stem cell growth factor, against radiation-induced gastrointestinal syndrome in mice. PLoS One 2009; 4: e8014.
17. Barker N, van Es J H, Kuipers J, et al. Identification of stem cells in small intestine and colon by marker gene Lgr5. Nature 2007; 449: 1003-7.
18. Ootani A, Li X, Sangiorgi E, et al. Sustained in vitro intestinal epithelial culture within a Wnt-dependent stem cell niche. Nat Med 2009; 15: 701-6.

### INCORPORATION BY REFERENCE

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

It should be understood that the drawings and detailed description herein are to be regarded in an illustrative rather than a restrictive manner, and are not intended to limit the invention to the particular forms and examples disclosed. On the contrary, the invention includes any further modifications, changes, rearrangements, substitutions, alternatives, design choices, and embodiments apparent to those of ordinary skill in the art, without departing from the spirit and scope of this invention, as defined by the following claims. Thus, it is intended that the following claims be interpreted to embrace all such further modifications, changes, rearrangements, substitutions, alternatives, design choices, and embodiments.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

1. A method for protecting, mitigating or otherwise treating radiation-induced depletion of tissue stem cells, comprising:
identifying a mammalian subject having, or expected to receive, radiation-induced depletion of somatic stem cells for least one tissue compartment or type;
administering to the subject a Wnt pathway activator/agonist in an amount sufficient to stimulate amplification of the surviving somatic stem cell pool for the at least one tissue compartment; and
administrating to the subject, subsequent to administering the a Wnt pathway activator/agonist, an amount of a CBP/catenin antagonist sufficient to promote differentiation of the amplified somatic stem cells, wherein a method for protecting, mitigating or otherwise treating radiation-induced depletion of the somatic stem cells for the at least one tissue compartment or type is afforded.

2. The method of claim 1, wherein the exposure to radiation is at least 6 Gy, at least 7 Gy, at least 8 Gy, at least 9 Gy, or at least 10 Gy.

3. The method of claim 1, wherein the somatic stem cells comprise at least one selected from the stem cell group consisting of skin including keratinocyte stem cells, epidermal, follicular, hematopoietic, mammary, intestinal epithelium including crypt cells, intestinal stem cell (ISC), mesenchymal including muscle satellite cells, melanocyte stem cells, osteoblasts and chondrocyte progenitors, endothelial, neural, including either the ependymal or the subventricular zone cells, neural crest, olfactory, testicular, uterine, lung, and cuticle stem cells.

4. The method of claim 3, wherein the somatic stem cells comprise intestinal stem cells (ISC).

5. The method of claim 4, comprising amplifying the surviving intestinal stem cell (ISC) pool, followed by accelerated differentiation of the amplified ISC in crypt-villi axis, providing for villous regeneration and intestinal restitution.

6. The method of claim 3, wherein treating radiation-induced gastrointestinal syndrome (RIGS) is afforded.

7. The method of claim 1, wherein the somatic stem cells comprise skin stem cells of a skin tissue compartment or type, and wherein enhanced post-radiation skin repair and/or homeostatic maintenance is provided at the skin tissue compartment or type.

8. The method of claim 1, wherein the somatic stem cells for the at least one tissue compartment or type comprise quiescent somatic stem cells, and wherein administering the CBP/catenin antagonist comprises CBP/catenin antagonist-mediated activation of the quiescent somatic stem cells to enhance or accelerate asymmetric renewing divisions relative to, or at the expense of symmetric divisions among the somatic stem cells of the at least one tissue compartment or type.

9. The method of claim 1, wherein administration of either or both of the Wnt pathway activator/agonist or/and the CBP/p-catenin antagonist comprises at least one of oral, intravenous, intramuscular, topical, gingival, buccal, and sub cutaneous administration.

10. The method of claim 1, wherein the Wnt pathway activator/agonist is at least one selected from the group consisting of R-spondin1 (R-spo1), direct Wnt/catenin activators, LiCl, GSK3-beta inhibitors including CHIR (e.g., CHIR-911), small molecule inducers of Rspo1, arylhydrdocarbon receptor (AHR) agonists, beta napthoflavone, indole-3-carbinol (I3C), high-affinity AhR ligands DIM and ICZ), formylindolo[3,2-¾]carbazoles, 6-formylindolo[3,2-¾]carbazole (FICZ) FICZ-derived indolo[3,2-¾]carbazole-6-carboxylic acid metabolites and sulfoconjugates, which induce the expression of Rspo1.

11. The method of claim 1, wherein the CBP/catenin antagonist is at least one selected from the group of compounds and salts thereof of Table 1, or another compound disclosed herein.

12. The method of claim 11, wherein the CBP/p-catenin antagonist comprises ICG-001 or an active derivative or variant thereof.

13. The method claim 1, comprising co-administration of or adjunct treatment with at least one other therapeutic agent.

14. The method of claim 13, comprising simultaneously or adjunctively treating the subject with an anti-inflammatory agent or antiviral agent.

15. The method of claim 14, wherein said anti-inflammatory agent comprises a steroid or glucocorticoid steroid.

16. The method of claim 14, wherein the at least one anti-inflammatory agent is selected from the group consisting of: short-acting p2-agonists, long-acting p2-agonists, anticholinergics, corticosteroids, systemic corticosteroids, mast cell stabilizers, leukotriene modifiers, methylxanthines, p2-agonists, albuterol, levalbuterol, pirbuterol, artformoterol, formoterol, salmeterol, anticholinergics including ipratropium and tiotropium; corticosteroids including beclomethasone, budesonide, flunisolide, fluticasone, mometasone, triamcinolone, methyprednisolone, prednisolone, prednisone; leukotriene modifiers including montelukast, zafirlukast, and zileuton; mast cell stabilizers including cromolyn and nedocromil; methylxanthines including theophylline; combination drugs including ipratropium and albuterol, fluticasone and salmeterol, glucocorticoid steroids, budesonide and formoterol; antihistamines including hydroxyzine, diphenhydramine, loratadine, cetirizine, and hydrocortisone; immune system modulating drugs including tacrolimus and pimecrolimus; cyclosporine; azathioprine; mycophenolatemofetil; and combinations thereof.

17. The method of claim 13, wherein the one additional therapeutic agent is selected from the group consisting of anti-microbial agents, antifungal agents, and antibiotic agents.

18. The method of claim 17, wherein, the at least one additional therapeutic agent is selected from the group consisting of: ciclosporin, hyaluronic acid, carmellose, macrogol(s), dextran and hyprolose, sodium and calcium, sodium and povidone, hypromellose, carbomer, amikacin, gentamicin, kanamycin, neomycin, netilmicin, streptomycin, tobramycin, paromomycin, geldanamycin, herimycin, loracarbef, ertapenem, imipenem/cilastatin, meropenem, cefadroxil, cefazolin, cefalotin/cefalothin, cephalexin, cefaclor, cefamandole, cefoxitin, cefuroxime, cefixime, cefdinir, cefditoren, cefoperazone, cefotaxime, cefpodoxime, ceftazidime, ceftibuten, ceftizoxime, ceftriaxone, cefeprime, teicoplanin, vancomycin, azithromycin, clarithromycin, dirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, nafcillin, penicillin, peperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, protosil, sulfacetamide, sulfamethizole, sulfanilamide, sulfasalazine, sulfisoxazole, trimethoprim, trimethoprim-sulfamethoxazole, demeclocycline, doxycycline, minocycline, oxytetracycline, tetracycline, arsphenamine, chloramphenicol, clindamycin, lincoamycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin/rifampicin, imidazole, miconazole, ketoconazole, clotrimazole, econazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, teronazole, terbinafine, amorolfine, naftifme, butenafme, anidulafungin, caspofungin, micafungin, ciclopirox, flucytosine, griseofulvin, Gentian violet, haloprogin, tolnaftate, undecylenic acid, and combinations thereof.

19. The method of claim 1, wherein the mammalian subject is a cancer patient that received or will receive high dose radiation therapy.

20. The method of claim 1, comprising treatment of at least one condition selected from the group consisting of radiation-induced pulmonary syndrome (RIPS); radiation induced bone marrow syndrome (RIBMS); radiation-induced bladder injury, radiation-induced liver damage (RILD); radiation-induced salivary gland injury; radiation-induced kidney injury; acute or chronic radiation proctitis; radiation esophagitis; radiation-induced cutaneous ulcer and fibrosis; radiation-induced pharyngeal fibrosis and dysfunction; chronic radiation-induced mucosal ulcers and fistulae; and chemotherapy-induced mucositis.

21. The method of claim 1, wherein the mammalian subject is a cancer patient that received or will receive radiation therapy and chemotherapy.

22. The method of claim 21, wherein the mammalian subject is a cancer patient that received or will receive high dose radiation therapy and chemotherapy.


SEQUENCE LISTING (12 sequences)

SEQ ID NO: 1 - DNA, 2339 bp, Homo sapiens
cgggtcgacgatttcgtcgcgccctcgcccctcccgggcctgcccccgtcgcgactggcagcacgaagctgagattgtggtttcctggtgattcaggtgggagtgggccagaagatcaccgctggcaaggactggtgtttgtcaactgtaaggactcatggaacagatctaccagggattctcagaccttagtttgagaaatgctgcaattaaaggcaaatcctatcactctgagtgatcgctttggtgtcgaggcaatcaaccataaagataaatgcaaatatggaaattgcataacagtactcagtattaaggttggtttttggagtagtccctgctgacgtgacaaaaagatctctcatatgatattccgaggtatctttgaggaagtctctctttgaggacctccctttgagctgatggagaactgggctccccacaccctctctgtccccagctgagattatggtggatttgggctacggcccaggcctgggcctcctgctgctgacccagccccagaggtgttagcaagagccgtgtgctatccaccctccccgagaccacccctccgaccaggggcctggagctggcgcgtgactatgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctagggacactgtccagcctccaggcccatgcagaaagagttcagtgctactctgcgtgattcaagctttcctgaactggaacgtcgggggcaaagcatacacacacactccaatccatccatgcatacacagacacaagacacacacgctcaaacccctgtccacatatacaaccatacatacttgcacatgtgtgttcatgtacacacgcagacacagacaccacacacacacatacacacacacacacacacgcacacctgaggccaccagaagacacttccatccctcgggcccagcagtacacacttggtttccagagctcccagtggacatgtcagagacaacacttcccagcatctgagaccaaactgcagaggggagccttctggagaagctgctgggatcggaccagccactgtggcagatgggagccaagcttgaggactgctggtggcctgggaagaaaccttcttcccatcctgttcagcactcccagctgtgtgactttatcgttggagagtattgttaccttccaggatacatatcagggttaacctgactttgaaaactgcttaaaggtttatttcaaattaaaacaaaaaaatcaacgacagcagtagacacaggcaccacattcctttgcagggtgtgagggtttggcgaggtatgcgtaggagcaagaagggacagggaatttcaagagaccccaaatagcctgctcagtagagggtcatgcagacaaggaagaaaacttaggggctgctctgacggtggtaaacaggctgtctatatccttgttactcagagcatggcccggcagcagtgttgtcacagggcagcttgttaggaatgataatctcaggtctcattccagacctggagagccatgagtctaaattttaagattcctgatgattggcatgttacccaaatttgagaagtgct

SEQ ID NO: 2 - protein, 263 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 3 - DNA, 3089 bp, Homo sapiens
attccctccctggtgctcgcagaggactggcccctctccgggctgggagctccggccgagcggaggcgcgacggagagcaccagcgcagggcagagagcccggagcgaccggccagagtagggcatccgctcgggtgctgcggagaacgagggcagctccgagccgccccggaggaccgatgcgccgggtggggcgctggccccgagggcgtgagccgtccgcagattgagcaacttgggaacgggcgggcggagcgcaggcgagccgggcgcccaggacagtcccgcagcgggcgggtgagcgggccgcgccctcgcccctcccgggcctgcccccgtcgcgactggcagcacgaagctgagattgtggtttcctggtgattcaggtgggagtgggccagaagatcaccgctggcaaggactggtgtttgtcaactgtaaggactcatggaacagatctaccagggattctcagaccttagtttgagaaatgctgcaattaaaggcaaatcctatcactctgagtgatcgctttggtgtcgaggcaatcaaccataaagataaatgcaaatatggaaattgcataacagtactcagtattaaggttggtttttggagtagtccctgctgacgtgacaaaaagatctctcatatgatattccgaggtatctttgaggaagtctctctttgaggacctccctttgagctgatggagaactgggctccccacaccctctctgtccccagctgagattatggtggatttgggctacggcccaggcctgggcctcctgctgctgacccagccccagaggtgttagcaagagccgtgtgctatccaccctccccgagaccacccctccgaccaggggcctggagctggcgcgtgactatgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctagggacactgtccagcctccaggcccatgcagaaagagttcagtgctactctgcgtgattcaagctttcctgaactggaacgtcgggggcaaagcatacacacacactccaatccatccatgcatacatagacacaagacacacacgctcaaacccctgtccacatatacaaccatacatacttgcacatgtgtgttcatgtacacacgcagacacagacaccacacacacacatacacacacacacacacacacacctgaggccaccagaagacacttccatccctcgggcccagcagtacacacttggtttccagagctcccagtggacatgtcagagacaacacttcccagcatctgagaccaaactgcagaggggagccttctggagaagctgctgggatcggaccagccactgtggcagatgggagccaagcttgaggactgctggtgacctgggaagaaaccttcttcccatcctgttcagcactcccagctgtgtgactttatcgttggagagtattgttacccttccaggatacatatcagggttaacctgactttgaaaactgcttaaaggtttatttcaaattaaaacaaaaaaatcaacgacagcagtagacacaggcaccacattcctttgcagggtgtgagggtttggcgaggtatgcgtaggagcaagaagggacagggaatttcaagagaccccaaatagcctgctcagtagagggtcatgcagacaaggaagaaaacttaggggctgctctgacggtggtaaacaggctgtctatatccttgttactcagagcatggcccggcagcagtgttgtcacagggcagcttgttaggaatgagaatctcaggtctcattccagacctggtgagccagagtctaaattttaagattcctgatgattggcatgttacccaaatttgagaagtgctgctgtaattccccttaaaggacgggagaaagggccccggccatcttgcagcaggagggattctggtcagctataaaggaggactttccatctgggagaggcagaatctatatactgaagggctagtggcactgccaggggaagggagtgcgtaggcttccagtgatggttggggacaatcctgcccaaaggcagggcagtggatggaataactccttgtggcattctgaagtgtgtgccaggctctggactaggtgctaggtttccagggaggagccaaacacgggccttgctcttgtggagcttagaggttggtggggaagaaaataggcatgcaccaaggaattgtacaaacacatatataactacaaaaggatggtgccaagggcaggtgaccactggcatctatgcttagctatgaaagtgaataaagcagaataaaaataaaatactttctctcagg

SEQ ID NO: 4 - protein, 263 aa, Homo sapiens; identical to SEQ ID NO: 2

SEQ ID NO: 5 - DNA, 2910 bp, Homo sapiens
attccctccctggtgctcgcagaggactggcccctctccgggctgggagctccggccgagcggaggcgcgacggagagcaccagcgcagggcagagagcccggagcgaccggccagagtagggcatccgctcgggtgctgcggagaacgagggcagctccgagccgccccggaggaccgatgcgccgggtggggcgctggccccgagggcgtgagccgtccgcagattgagcaacttgggaacgggcgggcggagcgcaggcgagccgggcgcccaggacagtcccgcagcgggcgggtgagcgggccgcgccctcgcccctcccgggcctgcccccgtcgcgactggcagcacgaagctgagattgtggtttcctggtgattcaggtgggagtgggccagaagatcaccgctggcaaggactgggttggtttttggagtagtccctgctgacgtgacaaaaagatctctcatatgatattccgaggtatctttgaggaagtctctctttgaggacctccctttgagctgatggagaactgggctccccacaccctctctgtccccagctgagattatggtggatttgggctacggcccaggcctgggcctcctgctgctgacccagccccagaggtgttagcaagagccgtgtgctatccaccctccccgagaccacccctccgaccaggggcctggagctggcgcgtgactatgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctagggacactgtccagcctccaggcccatgcagaaagagttcagtgctactctgcgtgattcaagctttcctgaactggaacgtcgggggcaaagcatacacacacactccaatccatccatgcatacatagacacaagacacacacgctcaaacccctgtccacatatacaaccatacatacttgcacatgtgtgttcatgtacacacgcagacacagacaccacacacacacatacacacacacacacacacacacctgaggccaccagaagacacttccatccctcgggcccagcagtacacacttggtttccagagctcccagtggacatgtcagagacaacacttcccagcatctgagaccaaactgcagaggggagccttctggagaagctgctgggatcggaccagccactgtggcagatgggagccaagcttgaggactgctggtgacctgggaagaaaccttcttcccatcctgttcagcactcccagctgtgtgactttatcgttggagagtattgttacccttccaggatacatatcagggttaacctgactttgaaaactgcttaaaggtttatttcaaattaaaacaaaaaaatcaacgacagcagtagacacaggcaccacattcctttgcagggtgtgagggtttggcgaggtatgcgtaggagcaagaagggacagggaatttcaagagaccccaaatagcctgctcagtagagggtcatgcagacaaggaagaaaacttaggggctgctctgacggtggtaaacaggctgtctatatccttgttactcagagcatggcccggcagcagtgttgtcacagggcagcttgttaggaatgagaatctcaggtctcattccagacctggtgagccagagtctaaattttaagattcctgatgattggcatgttacccaaatttgagaagtgctgctgtaattccccttaaaggacgggagaaagggccccggccatcttgcagcaggagggattctggtcagctataaaggaggactttccatctgggagaggcagaatctatatactgaagggctagtggcactgccaggggaagggagtgcgtaggcttccagtgatggttggggacaatcctgcccaaaggcagggcagtggatggaataactccttgtggcattctgaagtgtgtgccaggctctggactaggtgctaggtttccagggaggagccaaacacgggccttgctcttgtggagcttagaggttggtggggaagaaaataggcatgcaccaaggaattgtacaaacacatatataactacaaaaggatggtgccaagggcaggtgaccactggcatctatgcttagctatgaaagtgaataaagcagaataaaaataaaatactttctctcagg

SEQ ID NO: 6 - protein, 263 aa, Homo sapiens; identical to SEQ ID NO: 2

SEQ ID NO: 7 - DNA, 2589 bp, Homo sapiens
attccctccctggtgctcgcagaggactggcccctctccgggctgggagctccggccgagcggaggcgcgacggagagcaccagcgcagggcagagagcccggagcgaccggccagagtagggcatccgctcgggtgctgcggagaacgagggcagctccgagccgccccggaggaccgatgcgccgggtggggcgctggccccgagggcgtgagccgtccgcagattgagcaacttgggaacgggcgggcggagcgcaggcgagccgggcgcccaggacagtcccgcagcgggcgggtgagcgggccgcgccctcgcccctcccgggcctgcccccgtcgcgactggcagcacgaagctgagattgtggtttcctggtgattcaggtgggagtgggccagaagatcaccgctggcaaggactgggttggtttttggagtagtccctgctgacgtgacaaaaagatctctcatatgatattccgagtcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctagggacactgtccagcctccaggcccatgcagaaagagttcagtgctactctgcgtgattcaagctttcctgaactggaacgtcgggggcaaagcatacacacacactccaatccatccatgcatacatagacacaagacacacacgctcaaacccctgtccacatatacaaccatacatacttgcacatgtgtgttcatgtacacacgcagacacagacaccacacacacacatacacacacacacacacacacacctgaggccaccagaagacacttccatccctcgggcccagcagtacacacttggtttccagagctcccagtggacatgtcagagacaacacttcccagcatctgagaccaaactgcagaggggagccttctggagaagctgctgggatcggaccagccactgtggcagatgggagccaagcttgaggactgctggtgacctgggaagaaaccttcttcccatcctgttcagcactcccagctgtgtgactttatcgttggagagtattgttacccttccaggatacatatcagggttaacctgactttgaaaactgcttaaaggtttatttcaaattaaaacaaaaaaatcaacgacagcagtagacacaggcaccacattcctttgcagggtgtgagggtttggcgaggtatgcgtaggagcaagaagggacagggaatttcaagagaccccaaatagcctgctcagtagagggtcatgcagacaaggaagaaaacttaggggctgctctgacggtggtaaacaggctgtctatatccttgttactcagagcatggcccggcagcagtgttgtcacagggcagcttgttaggaatgagaatctcaggtctcattccagacctggtgagccagagtctaaattttaagattcctgatgattggcatgttacccaaatttgagaagtgctgctgtaattccccttaaaggacgggagaaagggccccggccatcttgcagcaggagggattctggtcagctataaaggaggactttccatctgggagaggcagaatctatatactgaagggctagtggcactgccaggggaagggagtgcgtaggcttccagtgatggttggggacaatcctgcccaaaggcagggcagtggatggaataactccttgtggcattctgaagtgtgtgccaggctctggactaggtgctaggtttccagggaggagccaaacacgggccttgctcttgtggagcttagaggttggtggggaagaaaataggcatgcaccaaggaattgtacaaacacatatataactacaaaaggatggtgccaagggcaggtgaccactggcatctatgcttagctatgaaagtgaataaagcagaataaaaataaaatactttctctcagg

SEQ ID NO: 8 - protein, 236 aa, Homo sapiens
MIFRVSAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 9 - DNA, 2721 bp, Homo sapiens
attccctccctggtgctcgcagaggactggcccctctccgggctgggagctccggccgagcggaggcgcgacggagagcaccagcgcagggcagagagcccggagcgaccggccagagtagggcatccgctcgggtgctgcggagaacgagggcagctccgagccgccccggaggaccgatgcgccgggtggggcgctggccccgagggcgtgagccgtccgcagattgagcaacttgggaacgggcgggcggagcgcaggcgagccgggcgcccaggacagtcccgcagcgggcgggtgagcgggccgcgccctcgcccctcccgggcctgcccccgtcgcgactggcagcacgaagctgagattgtggtttcctggtgattcaggtgggagtgggccagaagatcaccgctggcaaggactgggttggtttttggagtagtccctgctgacgtgacaaaaagatctctcatatgatattccgaggtatctttgaggaagtctctctttgaggacctccctttgagctgatggagaactgggctccccacaccctctctgtccccagctgagattatggtggatttgggctacggcccaggcctgggcctcctgctgctgacccagccccagaggtgttagcaagagccgtgtgctatccaccctccccgagaccacccctccgaccaggggcctggagctggcgcgtgactatgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctagggacactgtccagcctccaggcccatgcagaaagagttcagtgctactctgcgtgattcaagctttcctgaactggaacgtcgggggcaaagcatacacacacactccaatccatccatgcatacatagacacaagacacacacgctcaaacccctgtccacatatacaaccatacatacttgcacatgtgtgttcatgtacacacgcagacacagacaccacacacacacatacacacacacacacacacacacctgaggccaccagaagacacttccatccctcgggcccagcagtacacacttggtttccagagctcccagtggacatgtcagagacaacacttcccagcatctgagaccaaactgcagaggggagccttctggagaagctgctgggatcggaccagccactgtggcagatgggagccaagcttgaggactgctggtgacctgggaagaaaccttcttcccatcctgttcagcactcccagctgtgtgactttatcgttggagagtattgttacccttccaggatacatatcagggttaacctgactttgaaaactgcttaaaggtttatttcaaattaaaacaaaaaaatcaacgacagcagtagacacaggcaccacattcctttgcagggtgtgagggtttggcgaggtatgcgtaggagcaagaagggacagggaatttcaagagaccccaaatagcctgctcagtagagggtcatgcagacaaggaagaaaacttaggggctgctctgacggtggtaaacaggctgtctatatccttgttactcagagcatggcccggcagcagtgttgtcacagggcagcttgttaggaatgagaatctcaggtctcattccagacctggtgagccagagtctaaattttaagattcctgatgattggcatgttacccaaatttgagaagtgctgctgtaattccccttaaaggacgggagaaagggccccggccatcttgcagcaggagggattctggtcagctataaaggaggactttccatctgggagaggcagaatctatatactgaagggctagtggcactgccaggggaagggagtgcgtaggcttccagtgatggttggggacaatcctgcccaaaggcagggcagtggatggaataactccttgtggcattctgaagtgtgtgccaggctctggactaggtgctaggtttccagggaggagccaaacacgggccttgctcttgtggagcttagaggttggtggggaagaaaataggcatgcaccaaggaattgtacaaacacatatataactacaaaaggatggtgccaagggcaggtgaccactggcatctatgcttagctatgaaagtgaataaagcagaataaaaataaaatactttctctcagg

SEQ ID NO: 10 - protein, 200 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGQKRRKGGQGRRENANRNLARKESKEAGAGSRRRKGQQQQQQQGTVGPLTSAGPA

SEQ ID NO: 11 - DNA, 825 bp, Artificial Sequence
  Human Rspo1 recombinant variant with StrepII tag (ESAWSHPQFEK)
atgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgccgaaagcgcctggagccacccccagttcgagaagtga

SEQ ID NO: 12 - protein, 274 aa, Artificial Sequence
  human Rspo1 protein variant with SrepII tag (ESAWSHPQFEK) at c-terminus
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPAESAWSHPQFEK